SEQUENCE LISTING (101 sequences)

SEQ ID NO: 1 - protein, 833 aa, Komagataella phaffii
MAWNSKKRKDDRYESYKDFEDEDELNDETFGSDVYVGKNFDFSGGKPKANVPSAPAQPPNMLYAQAARTPVEDVLKPMASLWSEPSSQSKPQQQAPSQPTEKVLSLEEIEAQLLGGSVSTGPEAQQSPFYGYPPNIHPGQNQQFMQQFPNGVPLGFMVPPFNQSYYGPPDGQYQPSTVQHQGISQMQPPPQGVSQSVQPFPVSQLSNSVPPHVMQMPQVSTEGYSNAGPEGLAQTDSHSPTNEGQLDKERKAEPQDTLLDEEQQRPVGSTKERKTFDLSSLPALGSEEAHQRRIALQRQHHHPTEPVENYSKYENRQNHQLRSHHRHEDRFYNLDDPNLSPKERERLARRNHKLERILRGSGFMTPKDKDYCTQSQLSQIVTDDPYNEDFYYQVYKVLNNPNANGDTNVIAQKYLEQSGHKLGGKDQRVDVALQRMQHQVSLAVTVVRNRNAKKAEKSKSEQHPVFGKTTSATSKKPRQQLDVGKSLIDRVAEGKEDDTYEEREGSAKDITIKKTLSPIELKFSKSSQTFQLYLIEKIFDEILKLESQEREGVDITDNSLWEALNLEPMGTQQINNAEEIPSFISILYYEKGMKLFPRTFHFLSNEHRLILVNLIFNHLQKLYVILKGSYKIYESNDYQVPSEINNSIEQFQGTVLKSLVLFLSEAKFQDILESLIVLLNNNNLLFLCTTKVGLSIITILISRLELIKQELSQTLSGEDLSTWSSVYDNLFQSLEGRLSSIFPPKSNQQLADDSHVWQFLASLTLAGRLNHQRIIVGEVRTQIFDVVDQVKNLRATNDPVKVEKSNILLSNLNLFLNVMGLNATENDITELKS

SEQ ID NO: 2 - DNA, 2502 bp, Komagataella phaffii
  source 1..2502: mol_type = other DNA
atggcctggaattcaaagaaaagaaaggatgaccgatatgagtcctataaggattttgaggatgaagatgagttgaatgacgagacatttggcagtgatgtttatgttggtaaaaactttgatttttctggcggcaaaccaaaagcaaatgtgcctagcgccccagctcaacctccgaatatgttgtatgcccaggcagctcgaactccagtagaagatgtgttgaaaccaatggcttcgttgtggtcggaaccaagctctcaatccaaaccgcaacaacaagctccgtctcaacctacagagaaagtgttgtcattagaggaaatcgaggctcagcttttgggcgggtccgtttctacaggtcctgaagctcaacaatccccattttatggctatcctccgaacattcatcctgggcaaaatcagcagttcatgcagcaattccccaatggcgttcctcttggattcatggtgcctccatttaatcagtcatactatggaccccctgatggccagtatcagccctctactgttcaacatcagggcatttctcagatgcagccaccaccgcagggagtttctcaatcagttcagcccttcccagtttcccaactatctaactcggtacctcctcatgttatgcaaatgcctcaagtttcaaccgaaggctattctaacgcaggacctgaggggctagcccagactgattctcattcgcctacaaacgaaggccaattggataaagaacgtaaagccgagccacaggatactttgctggatgaagaacagcaacgtcctgtcgggtctactaaagaaagaaaaacttttgacttatcgagcttgccggctctcggatccgaggaagctcatcagagaagaattgcccttcagaggcagcatcaccaccctaccgagcccgttgagaactactcgaaatacgaaaaccgccagaatcatcagttgcgttctcaccatcgccatgaagatcgattttataaccttgatgatcctaacttgtcgcctaaggaacgagagcgtttagcaagaagaaaccataagctggagagaattctacgtggctccgggttcatgactcctaaggataaagactattgcactcaaagccaattgagtcagattgtgacagacgatccttataacgaagatttctactatcaggtttacaaagttctgaacaaccccaatgcgaacggggacacaaatgtaattgcacaaaaatatctcgaacagagtggtcacaaattaggtggtaaggaccaaagggttgatgttgcacttcaacggatgcagcatcaagtttcacttgcagttaccgttgttcggaatagaaatgctaagaaggccgaaaagagtaagagtgagcaacatccggtgtttggtaaaacaacttctgctacatcaaagaaacctagacaacaactcgatgttggcaagtctttgattgacagggtggccgaagggaaagaagatgatacttatgaagagcgagaaggatctgccaaagacattaccatcaagaaaacattatctcctattgaattgaagttctcaaaatccagtcaaacgtttcaactgtatttgattgaaaaaatatttgatgaaattttgaaactggaatctcaggagcgtgagggtgtcgacatcactgataattcactgtgggaagctttgaatttggagcccatgggcactcagcaaattaacaatgctgaagaaattccgtcttttatttcaatcctttactacgaaaagggtatgaagttgttcccacgtacgttccactttttgtccaacgagcatcgtcttatccttgtcaatttgatcttcaaccacttacaaaagctttatgttattttgaagggatcctacaagatatatgagagtaacgattatcaagttcctagtgagatcaacaactcaattgagcaattccagggtacagttttgaagtccttagtcctctttttatcagaggctaaatttcaagacattcttgaatcgcttatcgtactcttgaataacaacaacttattgtttctttgcactaccaaagttggactttcgattattaccattttgatttcacgactggaattgattaaacaggaattatctcagacgttaagtggagaagatttgtcaacatggagttcagtttatgataacttgttccaatctttggaaggacggttatcgtctatctttcctccaaaatcgaaccaacagctagcagacgactcacacgtttggcaatttttggcgtctttaactctggctggccgattgaaccatcaaaggattattgtgggagaagtccgaacacaaatcttcgatgtagttgaccaagtgaagaacctaagagccaccaacgatccagttaaggttgagaaatcaaatattcttttgtcaaatttgaaccttttcctcaacgtaatgggtttgaacgcaactgagaatgacatcactgaactaaaatcttaa

SEQ ID NO: 3 - protein, 845 aa, Komagataella pastoris
MAWNSKKRKDDRYDSYKDFEDEDELNDETFGSDVYVGKNFDFSGGKPKTNVPSASAQPSNMLYAQAARTPVEDVLKPMASLWSEASSQSKPQQQTPTQPAEKVLSLEEIEAQLLGGAVPSGAESQQSPFYGFPPNVNSGQNHQYMQQFPNGVPLGYMVPPFNQSYYGPPDGQYQPSAIQHQGISQMQPPPQGVPQSIPQSIPQSVPQSVQPFPVSPLSNSVPPHVMQVPQVSSDSYSNAGPEGLAQTNSTSPTNEGQSDKERKSESQNTLLDEEQQRPVGSTRGRKTFDLSTLPALGSEEAHQRRIALQRQHHHPNESVENYSRYENRQNHQLRSHHRHEDRFYNLDDSNLSPEERERLARRNHKLERILRGSGFMTPKDKDYCTQSQLSQIVTDDPYNEDFYYQVYKVLNNPNGNGDTNVIAQKYLEQSGHKLGGKDQRVDVALQRMQHQVSLAVTVVRNRNAKKAEKSKSEQHPVFGKTTSATSKKPRQQLDVGKSLIDRVAEGKEDDAFEEQEGSAKDITIKKTLSPIELKFSKSSQTFQLYLIEKTFDEILKLESQEREGVDITDSSLWEALNLEPMGTQQTNNAEEIPSFISILYYEKGMKLFPRTFHFLSNEHRLILVNLIFNHLQKLYVILKGSYKIYESNDYQVPNEINNSIEQFQGTVLKSLVLFLSEAKFQDILESLIVLLNNNNLLFLCTTKVGLSIITILISRLELIKQELSQTLSGEDLSTWSSVYDNLFQSLEGRLSSIFPPKSNQQLADDSHVWQFLASLTLAGRLNHQRIIVGEVRTQIFDVVDQVKNLRATNDPIRVEKSNILLSNLNLFLNVMGLNATENDITELKS

SEQ ID NO: 4 - protein, 796 aa, Saccharomyces cerevisiae
MSFFGLENSGNARDGPLDFEESYKGYGEHELEENDYLNDETFGDNVQVGTDFDFGNPHSSGSSGNAIGGNGVGATARSYVAATAEGISGPRTDGTAAAGPLDLKPMESLWSTAPPPAMAPSPQSTMAPAPAPQQMAPLQPILSMQDLERQQRQMQQQFMNFHAMGHPQGLPQGPPQQQFPMQPASGQPGPSQFAPPPPPPGVNVNMNQMPMGPVQVPVQASPSPIGMSNTPSPGPVVGATKMPLQSGRRSKRDLSPEEQRRLQIRHAKVEKILKYSGLMTPRDKDFITRYQLSQIVTEDPYNEDFYFQVYKIIQRGGITSESNKGLIARAYLEHSGHRLGGRYKRTDIALQRMQSQVEKAVTVAKERPSKLKDQQAAAGNSSQDNKQANTVLGKISSTLNSKNPRRQLQIPRQQPSDPDALKDVTDSLTNVDLASSGSSSTGSSAAAVASKQRRRSSYAFNNGNGATNLNKSGGKKFILELIETVYEEILDLEANLRNGQQTDSTAMWEALHIDDSSYDVNPFISMLSFDKGIKIMPRIFNFLDKQQKLKILQKIFNELSHLQIIILSSYKTTPKPTLTQLKKVDLFQMIILKIIVSFLSNNSNFIEIMGLLLQLIRNNNVSFLTTSKIGLNLITILISRAALIKQDSSRSNILSSPEISTWNEIYDKLFTSLESKIQLIFPPREYNDHIMRLQNDKFMDEAYIWQFLASLALSGKLNHQRIIIDEVRDEIFATINEAETLQKKEKELSVLPQRSQELDTELKSIIYNKEKLYQDLNLFLNVMGLVYRDGEISELK

SEQ ID NO: 5 - protein, 850 aa, Yarrowia lipolytica
MSFFGIAQPNQGNVQEYEFDDTYDDNLGGQLDESGDAFNDETFGGEASVGRDFDFHGETAKISYAIDEDQVAYERRAPQHAQMQHQQQQHQQRQAPPSLQPMASLWGAPDQKERIMGDEAQHGGQQAQQAHLQQQQHYQHVAAQQGGQQPLSLEQIEAQLMGRQAQAQGQQQQQHGVPPQGHAPPQGHFPPGYGYQGFPPGYPPPQGHPMQYQQPWQFPPLPNHDLNMQMMMHYQQQQQQQQQGQGQFQQPQFQGQSQGQPQVQVQQQQQQQQQPQAQTQGQPPAVASQPASQQQAAPPTAPLGPKEDKAIPTAPQGFQGPPHSGPYGYQPGPVHVSPNHTIPPMDENARLAKRNEMISKRKPHNNLMTGGDKDFVLRVQLQQMVTEDPYNEDFYYQVHSAINVRSNPNQPLDAFARTYLYQRGQRGRRARGNENGLQRLQQQVHKAVQFVKDHPKKDSIISQGALGKIHLGSNKGPRQALDLRKRDDTPDADNSTEAPTEGVSATPELRSATPVATTTTTTDAIRSITTSISKFNLQGGSYVSANDKRGILRAIENVYIVLLRLEAAERTQPENDLSDEFQVWKGEKDALVTQLWRELQITAPIDQADGSLHPFIAMLSHAKCKKAIPRVFRHISAEQRLTILTMIMAHIGDLDVVKNGHYSGEESNLPAKSREAIEVFSQTVLPPLVQYISEAEYGVIVGLLELMMDRNDTAAVVKTKVGSAFVTVLISRAELINQEGSVSAEDEETWNRIFNRVFGIVQRNNLNSLFPPRNVDDSYVWQFLASIALAAKIDHQRVMVDDVRDKIFGTMNEAKALPPDLSQQKINNLNLFLNVMGLNATTSEIGELQG

SEQ ID NO: 6 - protein, 797 aa, Ogataea polymorpha
MSFFGFDPSGPPSSGEKDQKLDTLDFEETYEGLGEQDEDAFNDETFGADVKEIGTNFDFTGGLKPASKPQTVDPNLSYAKAAQVDQVIAPMASLWNEEAKTASSASSQPASEQRVLSLEEIEAKLRQPQQQVPQMPQMNPYMYQQQQQLLVAAMASGQFPNPAAAAQALAQGYFPPFQPGVMPFMPFQAQQMMQQGALGQGQQPQQVHPPPAGVQVPQVPQVPQSAPAPVQQQQQQQPALQVPQALQSPPVPNAASPSSHSAQPVPQSVEHKVPSSASAPSSSAVSAPQSVPQAGSSDATGNFPPLGYDEKPRKQRPQRQNPENLTQEEREKLWRRQNKVSAIIRCSGFMNPRDKDFVTRVQLSQIVTDDPYNEDFYCQVYRVLNSSVGEDDMSSIAQKYLDQSGHRLGGREKRADVALQRMQQQVSKAVTVAKERGERTGVLAKEGALGKVSVGSGKQPRKQLVINTTGSSDEVVVPKENVYSKSSRAFQLSVIEDIYSQVLKLESLERENQERDSKDLWNALRLNDTIETSNTVVSPFISVLSIDKAMKVFNRLFHFLNPEQKIKLMEAIFANLHNIDVVMKGSYANYVNENYEIPKTEKSKIELFQMTVMKTLVLFVSESDFQQVLRFVVLIVKGGNNNVLFLSTTKIGLSLITILVSRLELIKQEFSAGLSPQDLAQWQTIYNHLFQALEGRLATIFPPYLSNDDARKVRNSESSSDDSYIWQFLASLSLAGLLSHQRIIVDEIRNEIFGSMGVSKDLKESGDLQGAEKHLSNLNLFLNVMGLKASESDITEL

SEQ ID NO: 7 - protein, 754 aa, Schizosaccharomyces pombe
MSFFGFNTTLPKENMFPNEGQLEEDGIDFEETYDDLGNQLNEAGDELNDETFGVSAGSIGRDFDFSGTTAQASAQLEDEQYQINQQNIFAKPVKPASSELPQVSRLNGASQFPSREPASTAINKLSDLQPMASIWENIVPEKPAIIPPEVASLQDRLGAQPSEKVFSLQELEEQLLNSMTAPKPPSQPAIPIVPSEMAAQVTRENISSLDPAISAASIGNVTFGQPNIPSTTTDFAGLAAPNMVHPSQAIPNPVMQPSLVPQMPYPQNGMYNPSVAPPASLVNLFQQEQLIQNQNLDEKRQKLERDHMLMAQCAGLMTRSDKSFIARIQISQLMSEDPESDDFYYRVYSIIRGRKPSEEEASHFIQTYLGPSNNRRRGRRSENPMQKLQQQLQRLVSSAKERPKATQLSLEGALGKIAVNTVRTPRQLLNVKRPTEPASSNSSLNNFSGFSTKKDVLHAIEKVYDLLLDFEQALRKASTLETTDQEQIDTWKTTLSEKLESIWKALYINESLEASSKTRPPFISIISHPKGMRLLPRLFPHLSKEQQISILKVVVYNFDSLDIVLRGTFDVNGELPLDVVSEMSSFTQFIIPPLLTIVNELDLETINNLFSQLLNRTNAVYLIQTKIGLSFLTLFISRAEILKQSGTVNQNEKEEWENTFNVMFNRVKGHFSTVFPPPNARAYADESYPWEFLAACATAASSEQHFTLVSETRDRVLDNIITSKRAPSEIAVVRISNVNLFLNAMGLDARQLSA

SEQ ID NO: 8 - protein, 843 aa, Trichoderma reesei
MSFFGFDTSGRHNAAAPGFSQPRDPFAGLSGHQEEGDALDFEDTYDGLGDQLDDADDAFNDDTFGGGAFGAGDSAAGKVGKDFDFFGQTAKVADAIEEEHLRFNRLQPAARANASQAQAPPQPSGISQQYAQQQYAQQQYSQQQPYYGEPVATRPARTGYEKYNEPDALPDLHVDQSIWGIGPPKAAAPAPKPAPQPAPSSSVGRKVMSLEEVEAAMRAQAKPSPQPPVSELASHQQVPAVYPSAQHASPLPQHGQAHPVTILQRPQGSQAKASPSPAPKLPTPHGQPEQYHNLHQQPAHPVHPVQILQNPKRLSSEAARAAPGSQELPQTAASAANLAQMSAHPQLAHMSEEEKVAFLDQETKRAKRNHKIWLLSKDNGVMTPQDKNFITRIQLQQLVSATGNPVEQGSDASLSEDFYYQVYSHIRAGQRQNPSQPLSNFAQTYLFQTGGRHGNMRRHGRPAENHIQRMEQQVQRAVEAAKNKPKNPQLVIAGSLGKISFSNAKTPKPLLNIKRAAENEPHHHRAGSGKKSPASSLDRKTILRNAELVYVTLMKIEDHVRVMPPPVTNQADQELQEKHKEWAATLEALNARLWQELKVHEPIGVIVPHPFIAFLSCAKGKKAIPRIFPHLSFEQRTTIMTMIVYHLDQLDVVNGAAVTNDETVPNARMRENIDLFLSTVMPSLMQYFNDTGLDIVDGVLNLIATNLNVDLIAKTRVGVSMLTLILSRAVLLKQTGGGTPEQWEKWDQTYETFFSRLEVSLPYMFPGSINTGSDVYVWQLLAALGVSASNDQQTRLVLAVKDRVIGTISLAKTLPPTMAAERLSSVNLFLHSIGLDVDMLMGQ

SEQ ID NO: 9 - protein, 870 aa, Kluyveromyces lactis
MSFFGFDATGRKPRERKQQGEDDSLDFEETYQGLGDYEPEEDDLLNDETFGTGADLGTDFDFGYGKGELDAGSGSGRAPAPAPVSYASIANAKMNRQADSKPGFEQSLDMQPMESLWGNSSSNQQQQVQPQAPPQFQAPPQVQQQGAPNVEGLAMQSLDRQQQMAMPPQPGFYGMQPPQNPGYGFIPQGFPQGQYQQFPGQNIPPVIPNQFGNAGFPNQPVNPQEQFQQVPPMMYNQTQGNIAHHMQHPQMAMPPMQNQPPVQPQQPHQQQLPPQYLPQQHQQDQQHQQQHQQQQVFQQPHSVDTAAITQAARSTQDYEHSNSPTPVPTESEQVPTPPHTPREYRRGPRRFGTQDPLTDEELRRLQIRQSKVDKILRHSGVMTPRDKDFITRYQLSQIVTDDPYNEDFYFQVYKIIQRGVTPSESNKDSIAKAYLEHSGHRLGGRYKRTDVALQRMQSQVEKAVTVAKERPQKSKGGLDNAKEGVLGKISATRNSKAPRKQLQIPTHKEDSPALSSQSSSALDEVSESLQGVDINAKRRRSSYALSSSVDQRTVLSRSGGRKFILSLIETVYEEVLELEAQLRSGQQPDSSKLWEALHVEDHGFDVSPFISILSFDKGIKIMPRIMNFLSREEKIKLFNCFFRELSHLNVIVISSYKTTPLPTKDQLKKMELFQSVFLKIIVSFLSSSAEFLEVMSLLINVIQNNNVSFITTSRIGLNLITVLISRAALMKQDVSRGKLLSSVEVSTWSEVYDRLFTSLETKLTAIFPTPEYMQHAISVYEQDSTQYDQSYIWQFLASLALSGKLNHQRILIDEVRDEIFEVISKAEELNAPSQQDPKLAYQRNKFYQDLNLFLNVMGLVARDGEITELK

SEQ ID NO: 10 - protein, 837 aa, Aspergillus niger
MSFFGFNTTLPRDRAPAGEQRGIFDTPDPFAEVARARLAANFQSGEDDDVIDFEDTYDGLGDQLDDDQDAFNDDTFGGGFDTGPVGKDFDFFGKTAQVADVIGEEQIRYSLQKPGAPATAPSQPSADTALPDTTAQKPKRSGYEKYNDPGYIPDLQAKSSVWGMSKKPEQQLQQQQLQQQLQQLQQPTPQMVAQAKKMLSLEEVEAQLRRQGPTSLPAQIPLSLPQSMPEPTQPLQRAQMPPLPEGFPQLPPEILQAQFAKGVPPAQLLHQPPMVPEPYPLPPNLPIHMLQNANLPPQHMVPPQRQAMPPAAQRPQQQQPPLPQAPRGNNAQLPVITNSQQLMHLTEEQRVAYLMEDAKRAKRNHKIFLLSRGNGLMTPQDKNFITRIQLQQLVAAAGNVADSDAEAVLAEDFYYQVYSQIRGAPRQHPHQPLGHFAQTYLLQTGNRLGGHGSRRQFQSADNHMQRMQQQVQRAVEAAKQKPKNKQLIIEGSLGKISFSNAKTPKPMLNIKRPESSEGAKTVKKPHTDLSVSDRKSVLTNLESVYSTLMDMEDMERTMPPPPDENDPEAIQKHMEWRQKIRSLNQKLWRDLKVMEPIVPNTNTPHPFIAFLAYPKGKKAIPRIFRHIDQEQRVTILTMIVVHLDSLDVVRRAQPVPGETQPSLAVREAIELFSQAVMPSLLGYVNEAPFNIIIGLLGLVIAQTHVQMIARTRIGLGILTMLLSRAEIVKEAGQATERDWQQWVEKFNVLFDTLEPTFADIFPSSINAGDDMYVWQFLAAVGIGASPEQQQRLVIAVKDRVMETVTYSKTLPADMASARLGNVNLFMRAIGLDVELLG

SEQ ID NO: 11 - protein, 627 aa, Homo sapiens
MPTVSVLEYALPQRPPQGPEDDRDLSERALPRRSTSPIIGSPPVRAVPIGTPPKQMAVPSFTQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQMLPPAPGFRAFFSAPPSATPPPQQHPPGPGPHLQNLRSQAPMFRPDTTHLHPQHRRLLHQRQQQNRSQHRNLNGAGDRGSHRSSHQDHLRKDPYANLMLQREKDWVSKIQMMQLQSTDPYLDDFYYQNYFEKLEKLSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALMDDRKHKICSMYDNLRGKLPGQERPSDDHFVQIMCIRKGKRMVARILPFLSTEQAADILMTTARNLPFLIKKDAQDEVLPCLLSPFSLLLYHLPSVSITSLLRQLMNLPQSAATPALSNPHLTAVLQNKFGLSLLLILLSRGEDLQSSDPATESTQNNQWTEVMFMATRELLRIPQAALAKPISIPTNLVSLFSRYVDRQKLNLLETKLQLVQGIR

SEQ ID NO: 12 - protein, 770 aa, Homo sapiens
MFRYESLEDCPLDEDEDAFQGLGEEDEEIDQFNDDTFGSGAVDDDWQEAHERLAELEEKLPVAVNEQTGNGERDEMDLLGDHEENLAERLSKMVIENELEDPAIMRAVQTRPVLQPQPGSLNSSIWDGSEVLRRIRGPLLAQEMPTVSVLEYALPQRPPQGPEDDRDLSERALPRRSTSPIIGSPPVRAVPIGTPPKQMAVPSFTQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQMLPPAPGFRAFFSAPPSATPPPQQHPPGPGPHLQNLRSQAPMFRPDTTHLHPQHRRLLHQRQQQNRSQHRNLNGAGDRGSHRSSHQDHLRKDPYANLMLQREKDWVSKIQMMQLQSTDPYLDDFYYQNYFEKLEKLSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALMDDRKHKICSMYDNLRGKLPGQERPSDDHFVQIMCIRKGKRMVARILPFLSTEQAADILMTTARNLPFLIKKDAQDEVLPCLLSPFSLLLYHLPSVSITSLLRQLMNLPQSAATPALSNPHLTAVLQNKFGLSLLLILLSRGEDLQSSDPATESTQNNQWTEVMFMATRELLRIPQAALAKPISIPTNLVSLFSRYVDRQKLNLLETKLQLVQGIR

SEQ ID NO: 13 - protein, 870 aa, Cricetulus griseus
SEQIDNOPATORTHOLOGOFCRICETULUGRISEUSPROTEINPATHOMOLOGISOFORMXCRICETULUSGRISEUSACCESSIONNUMBERNCBIXPMFRYESLEDCPLDEDEDAFQGLGEEDEEIDQFNDDTFGSGAVDDDWQEAHERLAELEEKLPAAADEQTGNRERDEMDLLGDHEENLAERLSKMVIENELEDPAIMRAVQTRPVLQPEPRSLNSSIWDGSEVLRRIRGPLLAQVEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 14 - protein, 770 aa, Cricetulus griseus
MFRYESLEDCPLDEDEDAFQGLGEEDEEIDQFNDDTFGSGAVDDDWQEAHERLAELEEKLPAAADEQTGNRERDEMDLLGDHEENLAERLSKMVIENELEDPAIMRAVQTRPVLQPEPRSLNSSIWDGSEVLRRIRGPLLAQEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 15 - protein, 686 aa, Cricetulus griseus
MKMKMHFKAWEKKMKKLINSMMIHLGQVQLPEPRSLNSSIWDGSEVLRRIRGPLLAQVEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 16 - protein, 685 aa, Cricetulus griseus
MKMKMHFKAWEKKMKKLINSMMIHLGQVQLPEPRSLNSSIWDGSEVLRRIRGPLLAQEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 17 - protein, 695 aa, Cricetulus griseus
MDLLGDHEENLAERLSKMVIENELEDPAIMRAVQTRPVLQPEPRSLNSSIWDGSEVLRRIRGPLLAQEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 18 - DNA, 1092 bp, synthetic construct
  source 1..1092: mol_type = other DNA
atgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagacaggttcagctgcaggagtccggtggtggtctggttcaagccggtggttcattaagattgtcctgtgctgcctctggtagaactttcacttctttcgcaatgggttggtttagacaagcacctggaaaagagagagagtttgttgcttctatctccagatccggtactttaactagatacgctgactctgccaagggtagattcactatttctgttgacaacgccaagaacactgtttctttgcaaatggacaaccttaacccagatgacaccgcagtctattactgtgccgctgacttgcacagaccatacggtccaggaacccaaagatccgatgagtacgattcttggggtcagggaactcaagtcactgtctcttcaggtggtggatctggtggtggaggttcaggtggtggaggatccggtggtggtggttctggtggtggtggatctggtggaggtgaagttcaacttgtcgaatccggtggtgcacttgtccaacctggtggatctcttagactttcttgtgccgcctccggttttcctgttaaccgttactctatgcgttggtacagacaagcccctggaaaagaacgtgaatgggttgccggaatgtcctcagctggtgacagatcctcctacgaagattctgtgaagggacgtttcaccatctccagagatgacgcccgtaacaccgtttaccttcaaatgaactcccttaagcctgaggatactgccgtctactattgtaacgtgaatgtcggatttgaatactggggacagggaacccaagttactgtctcttccggtggacatcaccaccaccatcactaatag

SEQ ID NO: 19 - protein, 362 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRQVQLQESGGGLVQAGGSLRLSCAASGRTFTSFAMGWFRQAPGKEREFVASISRSGTLTRYADSAKGRFTISVDNAKNTVSLQMDNLNPDDTAVYYCAADLHRPYGPGTQRSDEYDSWGQGTQVTVSSGGGSGGGGSGGGGSGGGGSGGGGSGGGEVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGHHHHHH

SEQ ID NO: 20 - DNA, 1830 bp, synthetic construct
  source 1..1830: mol_type = other DNA
atgaagtgggttactttcatctccttgttgttcttgttctcctcagcttactccagaggtgttttcagaagagatgctcacaagtccgaggttgctcacagattcaaggacttgggtgaagagaacttcaaggctttggttttgatcgctttcgctcagtacttgcagcagtgtccattcgaggaccacgttaagttggttaacgaggttactgagttcgctaagacttgtgttgctgacgaatccgctgagaactgtgataagtccttgcacactttgttcggtgacaagttgtgtactgttgctactttgagagaaacttacggtgagatggctgactgttgtgctaagcaagagcctgagagaaacgagtgtttcttgcaacacaaggacgacaacccaaacttgccaagattggttagaccagaggttgacgttatgtgtactgctttccacgacaacgaagagactttcttgaagaagtacttgtacgagatcgctagaagacacccatacttctacgctccagagttgttgttcttcgctaagagatacaaggctgctttcactgagtgttgtcaggctgctgataaggctgcttgtttgttgccaaagttggacgagttgagagatgagggtaaggcttcttccgctaagcagagattgaagtgtgcttccttgcagaagttcggagagagagcttttaaggcttgggctgttgctagattgtcccagagattcccaaaggctgagttcgctgaggtttccaagttggttactgacttgactaaggttcacacagagtgttgtcacggtgacttgttggaatgtgctgatgacagagctgacttggctaagtacatctgtgagaaccaggattccatctcctccaagttgaaagaatgttgtgagaagcctttgttggagaagtcccactgtatcgctgaggttgaaaacgacgaaatgccagctgacttgccatctttggctgctgacttcgttgaatccaaggacgtctgcaagaactacgctgaggctaaggacgttttcttgggtatgttcttgtatgagtacgctagaagacatccagactactccgttgttttgttgttgagattggctaagacttacgagactactttggagaagtgttgtgctgctgctgacccacatgagtgttacgctaaggttttcgacgagttcaagccattggttgaggaaccacagaacttgatcaagcagaactgtgagttgttcgagcagttgggtgagtacaagttccagaacgctttgttggttagatacactaagaaggttccacaggtttccactccaactttggttgaggtttccagaaacttgggtaaggttggttccaagtgttgtaagcacccagaggctaagagaatgccatgtgctgaggactacttgtctgttgttttgaaccagttgtgtgtcttgcacgaaaagacaccagtttccgacagagttactaagtgttgtactgaatccttggttaacagaagaccttgtttctccgctttggaggttgacgagacttacgttccaaaagagttcaacgctgagactttcactttccacgctgacatctgtactttgtccgagaaagagagacagatcaagaagcagactgctttggttgagttggttaagcacaagccaaaggctacaaaagagcagttgaaggctgttatggacgacttcgctgctttcgttgagaaatgttgtaaggctgacgacaaagagacttgtttcgctgaagagggtaagaagttggttgctgcttcccaagctgctttgggtctgtaa

SEQ ID NO: 21 - protein, 609 aa, synthetic construct [sequence omitted]

SEQ ID NO: 22 - DNA, 1001 bp, synthetic construct
  source 1..1001: mol_type = other DNA
catgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 23 - DNA, 1000 bp, synthetic construct
  source 1..1000: mol_type = other DNA
gcttaaaggactccatttcctaaaatttcaagcagtcctctcaactaaatttttttccattcctctgcacccagccctcttcatcaaccgtccagccttctcaaaagtccaatgtaagtagcctgcaaattcaggttacaacccctcaattttccatccaagggcgatccttacaaagttaatatcgaacagcagagactaagcgagtcatcatcaccacccaacgatggtgaaaaactttaagcatagattgatggagggtgtatggcacttggcggctgcattagagtttgaaactatggggtaatacatcacatccggaactgatccgactccgagatcatatgcaaagcacgtgatgtaccccgtaaactgctcggattatcgttgcaattcatcgtcttaaacagtacaagaaactttattcatgggtcattggactctgatgaggggcacatttccccaatgattttttgggaaagaaagccgtaagaggacagttaagcgaaagagacaagacaacgaacagcaaaagtgacagctgtcagctacctagtggacagttgggagtttccaattggttggttttgaatttttacccatgttgagttgtccttgcttctccttgcaaacaatgcaagttgataagacatcaccttccaagataggctatttttgtcgcataaatttttgtctcggagtgaaaaccccttttatgtgaacagattacagaagcgtcctacccttcaccggttgagatggggagaaaattaagcgatgaggagacgattattggtataaaagaagcaaccaaaatcccttattgtccttttctgatcagcatcaaagaatattgtcttaaaacgggcttttaactacattgttcttacacattgcaaacctcttccttctatttcggatcaactgtattgactacattgatcttttttaacgaagtttacgacttactaaatccccacaaacaaatcaactgagaaaa

SEQ ID NO: 24 - DNA, 991 bp, synthetic construct
  source 1..991: mol_type = other DNA
gcaaggcaactgagaaattgaatagtggtttcaagcccgctgactttttgtattatctcaatgtcggtgtttcacagtccccagaagggggctttgccttcaagggagacggaagagacatcgtcaaccctggggagaagtatttcaaatggcgcaagttcgctaatttttacgattaagcagtgctgtatggggtagttaataaatcgggaatatccttctgacgtgactgtaacaaatctctttttacgtggtgcgcatactggacagaggcagagtctcaatttcttcttttgagacaggctactacagcctgtgattcctcttggtacttggatttgcttttatctggctccgttgggaactgtgcctgggttttgaagtatcttgtggatgtgtttctaacactttttcaatcttcttggagtgagaatgcaggactttgaacatcgtctagctcgttggtaggtgaaccgttttaccttgcatgtggttaggagttttctggagtaaccaagaccgtcttatcatcgccgtaaaatcgctcttactgtcgctaataatcccgctggaagagaagttcgaacagaagtagcacgcaaagctcttgtcaaatgagaattgttaatcgtttgacaggtcacactcgtgggctatgtacgatcaacttgccggctgttgctggagagatgacaccagttgtggcatggccaattggtattcagccgtaccactgtatggaaaatgagattatcttgttcttgatctagtttcttgccattttagagttgccacattcgtaggtttcagtaccaataatggtaacttccaaacttccaacgcagataccagagatctgccgatccttccccaacaataggagcttactacgccatacatatagcctatctattttcactttcgcgtgggtgcttctatataaacggttccccatcttccgtttcatactacttgaattttaagcactaaa

SEQ ID NO: 25 - DNA, 864 bp, synthetic construct
  source 1..864: mol_type = other DNA
cttttctttgggcaaggaaaaatcaagaaaaagcagaggttaaagttttcaggggaatggcaattgctttatatatgggagaaagttaactacgtcggtgctgtaggcgtagagagcgactggagaatgcgtgatgaggtcgtctcttttcgcccccccttggcggggtaaaaattgcactactgcagaattactacacccctattccgaggagacggagtgcgacaaaaatggtaaagttcaccctagtctgcgacttttaattgacggacaccggcgtttacatgcgaaaaaaactaaagtgcgcgcatttcacggccgaggggggtcccacttgggactgagagggggtgggatctgaaatcgaggaggtatcaagaccccccgtttctcaactccctaatcaaaaattacgaagtcctcgttggaaaggagttaaaataattaagcggggtcggacgccataccgaggttatcttgcaggcattttactaatattggaattcggagctcaacttgcaaccaggcagggtttagctatgtaatcaatgtaatcaatataataaagcactaccacatcgaaggtttgggagggaggccaatagtgtcccccacagggtgctgatatcgcgattcttgggtgaggagacacatatttcactcctctcaccaaccaaccaagcggctcctcgcaagatgatttatccgattatccggacactatactcccatccagtttgatgccgatttcatcgattgtcctaaataatccttaaatatgtatagaacggtaccctggggttacataatccttatttaataatccctcccccaccgcttttctttttttttcttcttattgtc

SEQ ID NO: 26 - DNA, 1000 bp, synthetic construct
  source 1..1000: mol_type = other DNA
aaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaaca

SEQ ID NO: 27 - DNA, 549 bp, synthetic construct
  source 1..549: mol_type = other DNA
aatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 28 - DNA, 1000 bp, synthetic construct
  source 1..1000: mol_type = other DNA
attactgttttgggcaatcctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaa

SEQ ID NO: 29 - DNA, 1000 bp, synthetic construct
  source 1..1000: mol_type = other DNA
gtcaactgcgtactcttttgtcgaatggactactgaatctgcctcgatagccactataggaaggtccatagaggccagtttttcaactagtcttggtggaaagaaaccgacaaagcctttcatggagtcaccgatactgaaaggttcaaacaaagaatgcttgggtagtctcttaatacccatggcaacgaaaaaggggtcttcattgttcaacatgaattcgtatccacctttaatgtagtcataaagctgctgaagttccgaatcagtgatggaactgtctacagtgacaatataggagttctcaatcaccttatatccagtcgaatatatctggatagggtcgggtctcactgtggaagattcaaatgggttagatccctgtaatttcagcgatggagactcagtatgatggggcaaggaaaacggcaattggatattcaattggtcaagagatggtatcaaaagcgagtgtgccagggtagccacggtagccactgatgctaatctgataattttcatttctggagtgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagcaaaaaaaaaaatctaag

SEQ ID NO: 30 - DNA, 704 bp, synthetic construct
  source 1..704: mol_type = other DNA
aaattaatccataagataaggcaaatgtgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaat

SEQ ID NO: 31 - DNA, 1000 bp, synthetic construct
  source 1..1000: mol_type = other DNA
agctcagattggaaatgatttttgatcctaccaagaagcctttgatttccagaatctccgctaagtaagtaacccccgcaaacgcatgcatccatgcaaacaaaatactaacaattttagccccgttgttgagaaacccagaaaattgaatgttcaaccaatccagacgatcaataagaaaaaaggcccaaaggctacttccaaacctgctgccgccaaacctgctccttcaaaagccggtcccaagggaggtaagaaggtgagaaagccaaagaagacagttgaagaattggatcaggaaatggctgactactttgaaaataagaattagcccaacaaaatatgtacaagtattatataaatgaatctacatggtgtgttttatttagatcctccaaaccaaggaaagaaactaaacttatctccggacttacgagtcaaataactatccgcagttccttggaactcagactttcttccataagcggtcatatcatctttggactgtgggaatcctggacgaatctttgaaatgtcataatcttgctctctatctccaagcacagcgtccggtaaatgctggttcttctttctcagatgaatcttggatttaacaaataaagccgtgcctatggctaatgtactcaaaaacaaagtctgcttccagaatttcgcaaacgatggaatgccatttcctgtaaatgtactcattgaacctatgtttgattaaagttggtgtgaagtcatcaaacgagagtaaaatcagatactcgtgcaccggccaaaattgactgagctaatctctgcaggcttgacatccgaacacaacaaataggcgacaaatcttaactatctaatcgtaggctatggtagaactttgtgggggtagaggaagactacaacagcaagacaaaacaaaagagtcatagtttgactctctgcttttttcttctttctcttctttttcttcctccatattcgttatttatttcgaactgga

SEQ ID NO: 32 - DNA, 1000 bp, synthetic construct
  source 1..1000: mol_type = other DNA
cagccattaatctcacctcagtttttgaatcagtagaatttttaatgaaacaaacggttggtatattatttgatagagttgccaaatttccaaagataaatttttcatcaggtaatatcctgaataccgtaacatagtgactattggaagacactgctatcatattatatttcggataaaaatccaaaccccagaccgacctcttgagtctcaactccaagtcagccgcaactttaattatccgtggattgggagctagtttggacaacgcatcagtataatataactttacggttccattatcagacgctattgcaagaacttcctttccattgatctcgccaatgcggcagtaattgatatcgtagggtaggtctggaaagacgctggcgcttgtgtcccattctgcaggaatctctggcacggtgctaatggtagttatccaacggagctgaggtagtcgatatatctggatatgccgcctataggataaaaacaggagagggtgaaccttgcttatggctactagattgttcttgtactctgaattctcattatgggaaactaaactaatctcatctgtgtgttgcagtactattgaatcgttgtagtatctacctggagggcattccatgaattagtgagataacagagttgggtaactagagagaataatagacgtatgcatgattactacacaacggatgtcgcactctttccttagttaaaactatcatccaatcacaagatgcgggctggaaagacttgctcccgaaggataatcttctgcttctatctcccttcctcatatggtttcgcagggctcatgccccttcttccttcgaactgcccgatgaggaagtccttagcctatcaaagaattcgggaccatcatcgatttttagagccttacctgatcgcaatcaggatttcactactcatataaatacatcgctcaaagctccaactttgcttgttcatacaattcttgatattcaca

SEQ ID NO: 33 - DNA, 1000 bp, synthetic construct
  source 1..1000: mol_type = other DNA
tggttccctctcggtccaataccaaaaatattatcaccatacaggtctcccttcgataccagtgcaaagttgaaccgtgggattaccttggaatctacaaaaatagtgtcactcacaagtttgtcatcaaccacgctgccgcttgcaaaggagaactgaacatgaaggttgttagggtttgttatattggaataagtggtggatttgttgaaggcgaacgcaccaaagctacatccgtcctgagcacactgtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttcta

SEQ ID NO: 34 - DNA, 1001 bp, synthetic construct
  source 1..1001: mol_type = other DNA
gatatcgatctacacttaatagtagatgacgaggcatctctccaataggtaccatatctggtgtttcttgtaatttaagaatctgttggtctatgaatgtagatttgtcatgaacaatgatatatgggtcaggaggacaagatggtttctctgagttgggttgttgaggtgcctggcaagacttcggagcgttgatatccccaagacttgtagtgaccgatagttgaagcgtgtgtttgcaggaacggcacatcaatgcaactttcgtaactttggaattgagagttgatgcactgatgacgatacccgaaattttgacgattttaccaatatgacttgaagacaagtctctcattgaaaccttattatcgttactaagcaaaacgagctgacaagaagggaaggtggtcggtatttcctcgttgttcaaatatatgattctcctggcaatatctgtgatggcctgttcaaaaagtggaatcatttctgcaggatcatctaccaactttttattgagctcctcattgaatacgattaagtggtcattttgaatcgtcagtaagtacttgtttacaagtaaattctgtctgagttgttctctgtagatgtactgattttccatacgaaactccaaaatgaacgaacggaatgccttaatgacctcactgaactggtcatcgttctgttctccgggaaggacacttgtgttaaagactgatgctctatcaaaggacattgcaacaaagtataaacggttgtgagcgggaaaaagatgtgtaggtaattgtcgtagatgagactgattcagtagaaaacgcgtcctgcactatttttttctttcttcattacatttcctaatcgggacaaaatgaatctaaagacgtggttatgtagtacacgcatcgataggctatccccataccaaaacactattttaccccatccttgacaggttataaatatgcgatagtatgagtatcttcaaattcagctgaaatatc

SEQ ID NO: 35 - DNA, 1000 bp, synthetic construct
  source 1..1000: mol_type = other DNA
aataaaaaaacgttatagaaagaaattggactacgatatgctccaatccaaattgtcaaaattgaccaccgaaaaagaacaattggaatttgacaagaggaacaactcactagattctcaaacggagcgtcacctagagtcagtttccaagtcaattacagaaagtttggaaacagaagaggagtatctacaattgaattccaaacttaaagtcgagctgtccgaattcatgtcgctaaggctttcttacttggaccccatttttgaaagtttcattaaagttcagtcaaaaattttcatggacatttatgacacattaaagagcggactaccttatgttgattctctatccaaagaggattatcagtccaagatcttggactctagaatagataacattctgtcgaaaatggaagcgctgaaccttcaagcttacattgatgattagagcaatgatataaacaacaattgagtgacaggtctactttgttctcaaaaggccataaccatctgtttgcatctcttatcaccacaccatcctcctcatctggccttcaattgtggggaacaactagcatcccaacaccagactaactccacccagatgaaaccagttgtcgcttaccagtcaatgaatgttgagctaacgttccttgaaactcgaatgatcccagccttgctgcgtatcatccctccgctattccgccgcttgctccaaccatgtttccgcctttttcgaacaagttcaaatacctatctttggcaggacttttcctcctgccttttttagcctcaggtctcggttagcctctaggcaaattctggtcttcatacctatatcaacttttcatcagatagcctttgggttcaaaaaagaactaaagcaggatgcctgatatataaatcccagatgatctgcttttgaaactattttcagtatcttgattcgtttacttacaaacaactattgttgattttatctggagaataatcgaacaaa

SEQ ID NO: 36 - DNA, 1000 bp, synthetic construct
  source 1..1000: mol_type = other DNA
attgttgtgaatactctccttcatttggatttcttggacttcggactctcttgatctctcttcgaaagttttaactctgttcatgtataattttacccgctgtaggtcgctcataataccatgagtatgcacatcttttactccattaactttcaggtatgcaaaatacaatgaagatagtatatagctcaaagaatttagcattttgcattgatctaattgtgacattttctctatgatatcatctagcttcttaaactcgagaatctcgtccaacgaggcagaaacattgtccagtcttacgtcaagattattcacgagtttctggaccgtatcaacgttttccatcttaagattacagtaagtatcgtccttttgaactgcaaaggtagaaaagttaatttttgatttggtagtacactatgaaacttgctcaccccaatctttcctcctgacaggttgatctttatccctctactaaattgccccaagtgtatcaagtagactagatctcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaat

SEQ ID NO: 37 - DNA, 1000 bp, synthetic construct
  source 1..1000: mol_type = other DNA
tatacggtctatccactttggaaacgatgtagttgaaacggggaagtaatagtggttcccaaacgacatgaagaggttatataagtttgcaagagggtgacaccattttagttgtggttcccgggtatttttttaatctttttagtctaagatagcctccccagatattaccgagttgggccatttggggcggtatcggtggtatctgatggtagcgcgtttttacatgcctgtgcattgaactggcaaagagtatactatcgtggggccctgaaggaggcagcaaatggaccgtcaattggttgatcagggactcaagacaggtattgagcttttcaaacaaaaagagtataggcgctgctacaaggcatttacttctactatcaatttcattgagaatgatcccgagttggccgccagctgtgtatctcaactgatatctctgttagattgtagggcagcctgtttggaaaagctagatcaattgaatatggccttgaaagatggtcttaaaatgatcaagagagagtgccacaactgcaagggttatttgagaacttgcaaaattttagacctacaagggaagatcagtgaggctttgtctacagcaagagaagggatctccataatagaaactagaagagatcaggataatcaatttagatattccaaggttcttttggaacaattaaaggaactgaaaaatgcactgaaaatcaaattggacaagaaaaatcagctacacttcaaagttttaaagtttgacgcaccagtgccttgtacaaagaaactaagattagtcactccaagaacaatagatccttccatttttttgccgatagagctagtgaagctgatctttcgcctgttgaatttctcagacatgtatgcctgtttattggtctcaacaaaatggaactcaattatatcctcatcaccggaactgtttcgaaaacttcagttgaaatcccaactgtccaacaaggcgttaaacaattgt

SEQ ID NO: 38 - DNA, 951 bp, synthetic construct
  source 1..951: mol_type = other DNA
  variation 578: note = n is (T)x wherein x is 14-16
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctangatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 39 - DNA, 1238 bp, synthetic construct
  source 1..1238: mol_type = other DNA
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgagatctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 40 - DNA, 1425 bp, synthetic construct
  source 1..1425: mol_type = other DNA
caaacatttgctccccctagtctccagggaaatgtaaaatatactgctaatagaaaacagtaagacgctcagttgtcaggataattacgttcgactgtagtaaaacaggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccacccttagatctcagggattcccactatttggtattctgatatgtttttcctgatatgcatcaaaactctaatctaaaacctgaatctccgctattttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 41 - DNA, 1000 bp, synthetic construct
  source 1..1000: mol_type = other DNA
gtaaatagcggcagcaatccagtaaccttttctgaatagcagagccttaactaaaataatggccagggtaaaaaattcgaaatttgacaccaaaaataaagacttgtcgttataagtcttaacaaagtccgcaattttggagctaacggtggcggttgctgggatattcaataatggtagaatgttgctgcgggtatatgacagagcgtgaaacacactgaacaaggtaaatggaacaacagcaattgcaatatgggggaggatagtcaagaacaaagcagcaatggcaaagtactgaatattctccaaagccaaaaggtccagtggtttcaacgacaaagtcttgttggtatagctttggaacaaaaggacaccgaaagactcgacagcgcccacaaatacagcgttgtagaagaacgaattgattgctccagagcttctaatagtcagaagataccccaaacctccgagcaacgttagcacatgacctaagaaccaggcgaagtgaagagtctggaataacgacacccagtcagtttttcctgagctcctggtgggattggtagaagcatttgatttgcttggagtggttttatttgaagatggtgttgaagccattgttgctaaagagtcggagttttgcttttagggtttgttaagcaaaggaggaaaaactgcgccgtttgaagtcccaggtagtttcgcgtgtgaggccagccagggaaagcttccttcggtacttttttttcttttgcaggttccggacggattaagcttcgggttatgaggggggcggtagccaattccggacacaatattgcgtcgcagctagtcaccccgccataaatatacgcaggattgaggtaataacatcgatagtcttagtaattaatacaattcagtggcgaatttggcaacatgacgtaaggcccactgttgtctataaaaggggatgaattttcatgtttttgaggcctcccggacaatttattgaactcaa

SEQ ID NO: 42 - DNA, 1000 bp, synthetic construct
  source 1..1000: mol_type = other DNA
tggactgttcaatttgaagtcgatgctgacgatgtcaagagagatgctcaattatatttgtcatttgctggttacactggaaacgctacttttgttggcggaaactctaccagtttggccgtccatgtaaacgatgtcgttctgggccgtgaccgtttcaacacgaacataaccaatgacaaatccacttacaggtctagttcatatggaggcaattggtaccttacttctttggatgtcccaagtggggctttaacgtctggtactaacaatgtctcgtttgtcactacaaactccgaggtaaataaaggattcttgtgggattctctcaagtttgtttggaagttgtaacaggtttataagcatatcgtgcgcttgtccacaattgaatcatttattgttgcgagatacatgaacaaagtgtgaactgggacccattactacaattcccacgcaaccgttgtttcaaagcccatattttttgacaattgtttcgttacacccccagtttgatgtacatcgcttgcaatgatgtgtgtcccggagtattttccatattcagcttgaattcgtatactcaaccaatatctgggggtatacttttatgtaacctatacaaatcaactatactatttcacctttcgaccaatcatctcccatcttgttaagttttgcttcctatatccctgaccctgacatcacccatgattccgctcaacggttctcctctacatcgtccctcttttggagagggtgttcagtttgacattcaaattaccccccgccatcacgcgcaaccgagaccgcacccccgaattttcacaaattaccccacaccctatactccaccactatgagggttattagaactgatcacgtataaataccaccgcaagttcccaagggatcgtgttcttcttctccaattgcaatcatatttctgactctttctagttcagattaattcctttacacttgcttttttcccttacctttatcc

SEQ ID NO: 43 - DNA, 1001 bp, synthetic construct
  source 1..1001: mol_type = other DNA
agaccagcagtttaactacgcaaatccacaggaatttctacatcacaataccaatggtaataccacgacgtcaaggaatggaaacgacgacttggaggaagacttcgtcaacctcttgcggagtacccgaggctaagacaataagaagaaaaaaaaaagaaaagcggtgggggagggattattaaataaggattatgtaaccccagggtaccgttctatacatatttaaggattatttaggacaatcgatgaaatcggcatcaaactggatgggagtatagtgtccggataatcggataaatcatcttgcgaggagccgcttggttggttggtgagaggagtgaaatatgtgtctcctcacccaagaatcgcgatatcagcaccctgtgggggacactattggcctccctcccaaaccttcgatgtggtagtgctttattatattgattacattgattacatagctaaaccctgcctggttgcaagttgagctccgaattccaatattagtaaaatgcctgcaagataacctcggtatggcgtccgaccccgcttaattattttaactcctttccaacgaggacttcgtaatttttgattagggagttgagaaacggggggtcttgatacctcctcgatttcagatcccaccccctctcagtcccaagtgggacccccctcggccgtgaaatgcgcgcactttagtttttttcgcatgtaaacgccggtgtccgtcaattaaaagtcgcagactagggtgaactttaccatttttgtcgcactccgtctcctcggaataggggtgtagtaattctgcagtagtgcaatttttaccccgccaagggggggcgaaaagagacgacctcatcacgcattctccagtcgctctctacgcctacagcaccgacgtagttaactttctcccatatataaagcaattgccattcccctgaaaactttaacctctgctttttcttgatttttccttgcccaaagaaaag

SEQ ID NO: 44 - DNA, 1000 bp, synthetic construct
  source 1..1000: mol_type = other DNA
aattgattaagttcagtgaaatttcaaaccgctatacacaacaggacaactttgagtttagaaaaatccgatgtagtgtaacggctagcacggtccgctttcaccgggcagacccgggttcgactcccggcatcggagtttatttttccatttcgttctttagagtattctcctcagcatgcccccctgaatttttccttttttccatgtgtcccatttttccactttttttacagttttcctcgtgatgttaattggctacacaaaagctgccacacgaaaccttaatcacgaaaaactatacagccttcactaatccgtagccccataatatgttgtccacgtgctgttgggtactacctgtagactctcataccccactccgtctttctccaacaattaacgcagtaccgagatttatcagcagactcaaattgggcaaactctgtatttttccttgcccgcataatttatgggtctcaggcctccacgtttcctgtttacttgaagaatattggctgcggaaaaagtggtaaggacaacccccttttaattggatccagtttttccgaaatgttccgatccgtacgtcatctccgaagccgtacattttcactcaatctacgtagctttggactcagcgctcctggaattgccaggacagtttacttgagttgatattcccttgtagattgtgtgcttctttttccaaaatttgaggcttcgtttgaaaagtggaatctggtcgctagatcacttcatgcctatttttcacggaaaaataagtggtactatgcaccccttaaacctaaagaaaaacggaaaaattaccccaaaacctggtgatgtttttcgcccctttctttttatccgagtttttcttttttcttgtctgccaaattcctctcctgaccttagcgtccccggaaaaaattaactacttaaggaccgaatgagccccagcttttccccttctcttcattattccccataatataat

SEQ ID NO: 45 - DNA, 1000 bp, synthetic construct
  source 1..1000: mol_type = other DNA
ctgcacaaccattgccagtaaggacgaagagaaggccccactacccaaaattcaggataacgtcttcataccatgcagcgacgcctacaagacgctgtcaagacatgccaacttcaacgaagtgaactttaacacattgatcgggaaattgaccaccaagggaatgctggttgaggctggaagcgttgccagtgtcctgagggaactggaccgaaagtttagtaatgcataagaggatatatataggaatgcagtaataatattagtacccattaagtgggctaagccattggaaggccgtctgactgatggtggtgttcttctcatttagatagtgcatttgcaactaccgtctgagattgagtttgatgtgaagctccagcgccaaaacagtataagaaccttatctccgcattattgttcttgcgtaaaagtttgtgtgaagaaacaggggtagttgcgcagattagttgtaatatgcgcataggatgggtcattgacttctttcctcgaaagagccacaccgttagctaaaaaaggacgcgcatctaccccaaaatagaatgtggggaaataggacgcgcaacttcctctcaatcactggacgtcagaaaaacaaatgcgcaatcgagtcaccctccgtgataccctccgtgataccccctctccgtctattctgacagcgtctccccatgacgtttcaatctacttagaaaagatttcgtttttttttccttcaattacacgatctcatcttctgcaagggtctggaggacatcaccaatctgcgactccataacttagtcctgagtttatatttacgcttcatctgatgagtaggaagaaaaagtttcacgaaattcccccgccaacttgcccttcggaataagcagccactctccttctgcccatagtaagcttgcgcgaggccccaacttggccagaaactttaaatatgccaaacaatctcccccaatctaagttctccctcttctaaaaa

SEQ ID NO: 46 - DNA, 859 bp, synthetic construct
  source 1..859: mol_type = other DNA
ggaatctgtattgttagaaagaacgagagttttttacggcgccgccatattgggccgtgtgaaaacagcttgaaaccccactactttcaaaggttctgttgctatacacgaaccatgtttaaccaacctcgcttttgacttgactgaagtcatcggttaacaatcaagtaccctagtctgtctgaatgctcctttccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 47 - DNA, 664 bp, synthetic construct
  source 1..664: mol_type = other DNA
ccatattcagtaggtgtttcttgcacttttgcatgcactgcggaagaattagccaatagcgcgtttcatatgcgcttttaccccctcttttgtcaagcgcaaaatgcctgtaagatttggtgggggtgtgagccgttagctgaagtacaacaggctaattccctgaaaaaactgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 48 - DNA, 493 bp, synthetic construct
  source 1..493: mol_type = other DNA
ctgcagatagacttcaagatctcagggattcccactatttggtattctgatatgtttttcctgatatgcatcaaaactctaatctaaaacctgaatctccgctattttttttttttttttgatgaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 49 - DNA, 370 bp, synthetic construct
  source 1..370: mol_type = other DNA
gaccccgttttcgtgacaaattaatttccaacggggtcttgtccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 50 - DNA, 328 bp, synthetic construct
  source 1..328: mol_type = other DNA
ccggataagagaattttgtttgattatccgttcggataaatggacgcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 51 - DNA, 283 bp, synthetic construct
  source 1..283: mol_type = other DNA
gcctgctccatatttttccggttattaccccacctggaagtgcccagaattttccggggattacggataatacggtggtctggattaattaatacgccaagtcttacattttgttgcagtctcgtgcgagtatgtgcaataataaacaagatgagccaatttattggattagttgcagcttgaccccgccatagctaggcatagccaagtgctatgggtgttagatgatgcacttggatgcagtgagttttggagtataaaagatccttaaaattccaccctt

SEQ ID NO: 52 - DNA, 491 bp, Komagataella phaffii
  source 1..491: mol_type = other DNA
cttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcacctgcaggcc

SEQ ID NO: 53 - DNA, 488 bp, Komagataella phaffii
  source 1..488: mol_type = other DNA
cttttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactatcaaaacaca

SEQ ID NO: 54 - DNA, 985 bp, Komagataella phaffii
  source 1..985: mol_type = other DNA
agggcatcattgaggtttccacaaaaggaagaaacatggatccagagacatcaacagagaggaaagcgggtagtgaagccgaagccacaacacagcccgatttggaagggagttcacaatcaaggtgagtccagccattttttttcttttttttttttttattcaggtgaacccacctaactatttttaactgggatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaa

SEQ ID NO: 55 - DNA, 975 bp, Komagataella phaffii
  source 1..975: mol_type = other DNA
gtagcttgggtaggacttgacaagtacggcttccgtggtcataccaaacgcctttgttaccgttggctatacctaatgaccaaggcatttgtggattataacggtatcgtagttgaaaaatatgacgtaaccactggtactagcccccacaaggttgatgctgaatacgggaatcaaggtgccgattttaaaggagtagccactgaagggtttggctgggtcaatgcctcttttattttgggattaacctacttagatgtccaaggcatccgtgcgataggcgccgttacgtcccctgatgtatttttcaggaagctcaaaccttgggaacgcgcaagttatggcctaaggccatgtaacgagatagtcaagtcaaactagaagtatacggtttccccgcagaaatagcagaaataggcgacaaatacatacaacattttcattgtgatagggggcggcggttcctaggagggacaacccccagaaaccttgtagactacgttttcacgacgatgggttattactgtaaaggaagaatatactacccaccagttgaatgtttgaacggatcaaaggtcgaagggagtacacggcccaaccaacgtagctaccggagaaagcaagactttcccaaaccaaatagctccgggtttcttctccggcaacccgtcagtttttgtgtggccggacaaaaattcgcaccctcagtctaattgaaaggtcgggctccgagctctaggcgtttgcgcatgtaatattgcatcccctcccatagataatactgcgcgaacacagggtgcaaattatgatgaccacacatgccagtgaccaaaacagttttttagtctttaaaaaccctcggaacttctgagtatataaaggcttctcatttcctacaagcaaacaaagaagaaacttccactttctaactttttatctatagactttagagttacaaccaacgaacaataacaa

SEQ ID NO: 56 - DNA, 699 bp, Komagataella phaffii
  source 1..699: mol_type = other DNA
cgcattatttgggtcggctactgtcattggttatgatggccttaccgagcacgaaggtggttggatagaccacaactggaaacagttgtacaaacagattgcattcatttttgcttgtattggatactcgatggccatcaccgctcttatctgtttcatcctcaaccgtattccatttttgcaactgcgagcttcagaagaggctgaggagaaaggtatggatgaggatcagattggagagttcgcttatgactacgtggaagtacgtcgtgattttttggcttggggatcaggcccaaacaatggcttcaaggagccggaagttctggatcaggtagttccggttaatgatttcagcagtgaccagaatgtgactaatcagaccaacgaatctgagaagcagtagagtaaatatagagatgatatttagtgtattctaatgcttatgtaatgtattaagcaaaaagttgtgtttatgagttagcatttgtcttagcaaacataaaattatgtcgacatttgcaacccgcatgtctagtgtttttagatcgatcttcgatgtgtagaataatgcctccacgtgatgccccgcgattttgttgggtcgcaatgcctccaacataaacccatcacgtataaaaagccctcttaaccctcccccctgtttcgtttgcttcatcacttaacctgaactatcaa

SEQ ID NO: 57 - protein, 20 aa, synthetic construct
  VARIANT 3: note = X at position 3 is either F or L
  VARIANT 16: note = X at position 16 is either A or T
MKXSTNLILAIAAASXVVSA

SEQ ID NO: 58 - protein, 36 aa, synthetic construct
  VARIANT 3: note = X at position 3 is either F or L
  VARIANT 16: note = X at position 16 is either A or T
MKXSTNLILAIAAASXVVSAAPVAPAEEAANHLHKR

SEQ ID NO: 59 - protein, 85 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 60 - DNA, 30 bp, synthetic construct
  source 1..30: mol_type = other DNA
gtagggtctccgatcggagccgattgacga

SEQ ID NO: 61 - DNA, 37 bp, synthetic construct
  source 1..37: mol_type = other DNA
gatgggtctcgccggggttgatttctattgtcacagg

SEQ ID NO: 62 - DNA, 35 bp, synthetic construct
  source 1..35: mol_type = other DNA
gataggtctccccgggtacgctgcaggtcgacaac

SEQ ID NO: 63 - DNA, 35 bp, synthetic construct
  source 1..35: mol_type = other DNA
gataggtctccaattagtggatctgatatcaccta

SEQ ID NO: 64 - DNA, 35 bp, synthetic construct
  source 1..35: mol_type = other DNA
gttcggtctccaattgtctcgatgttcaaattctg

SEQ ID NO: 65 - DNA, 30 bp, synthetic construct
  source 1..30: mol_type = other DNA
gatcggtctcgagctcgggtctggcaatct

SEQ ID NO: 66 - DNA, 23 bp, synthetic construct
  source 1..23: mol_type = other DNA
cagagatcaataccaaatacctg

SEQ ID NO: 67 - DNA, 17 bp, synthetic construct
  source 1..17: mol_type = other DNA
cagccaagaatggtgtg

SEQ ID NO: 68 - DNA, 18 bp, synthetic construct
  source 1..18: mol_type = other DNA
tgcgttgacgttggtgac

SEQ ID NO: 69 - DNA, 19 bp, synthetic construct
  source 1..19: mol_type = other DNA
gtccttcaccaccgacacc

SEQ ID NO: 70 - DNA, 15 bp, synthetic construct
  source 1..15: mol_type = other DNA
ggcgatggtgagaac

SEQ ID NO: 71 - DNA, 16 bp, synthetic construct
  source 1..16: mol_type = other DNA
ccgaacattcatcctg

SEQ ID NO: 72 - DNA, 1275 bp, synthetic construct
  source 1..1275: mol_type = other DNA
gtacgctgcaggtcgacaacccttaatataacttcgtataatgtatgctatacgaagttattaggtctagatcggtaccgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtgttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggtaccattcgagaacccttaatataacttcgtataatgtatgctatacgaagttattaggtgatatcagatccact

SEQ ID NO: 73 - DNA, 24 bp, synthetic construct
  source 1..24: mol_type = other DNA
cctgaggctttgttccacccatct

SEQ ID NO: 74 - DNA, 30 bp, synthetic construct
  source 1..30: mol_type = other DNA
ggaacatagtagtaccaccggacataacga

SEQ ID NO: 75 - DNA, 20 bp, synthetic construct
  source 1..20: mol_type = other DNA
agactttcactttccacgct

SEQ ID NO: 76 - DNA, 19 bp, synthetic construct
  source 1..19: mol_type = other DNA
caacgaaagcagcgaagtc

SEQ ID NO: 77 - DNA, 2061 bp, synthetic construct
  source 1..2061: mol_type = other DNA
atgaagatgaagatgcatttcaaggcctgggagaagaagatgaagaaattgatcaattcaatgatgatacatttgggtcaggtgcagttgcctgaacccagaagtctgaattctagcatctgggatggatctgaagttctgaggcgaatccgtggaccactacttgctcaggtagaaatgcctacggtgtctgtattagaatatgccttgcctcagaggcccccacagggcccagaagatgatcgagacctttctgagcgagctttaccaaggcgttctacatcacctgtcattggaagtcctcctgttagagccgttcctataggcaccccacctaagcagatggccgttcccagcttcaaccaacagattctgtgtccgaagcctgttcatgtacgacctccgatgccaccacgttatcctgctccctatggtgagaggatgtctccaaaccagctctgcagtgtcccgaattcgtcccttctgggccacccttttcctcctagtgttcctcctgtactcagtcccctgcagagagcacagcttcttggaggagcacagctacagcctggtcgtatgtctcccagccagtttgcacgagtccctggatttgttggtagccccctggctgccatgaatcctaagttgctacaagggcgagttggacagatacttcccccagcaccaggcttccgtgccttctttagtgctccaccccctgctacaccccctccccaacagcatcctcctggcccaggacctcacctgcaaaacttaagacctcaggccccaatgtttagaccagacacaactcaccttcatccacagcaccggcgcctgttgcatcagaggcagcagcagagtagaaatcagcatcggaatctcaatggtgctggagacagaggaagtcaccgggccaatcatcaagatcatctccggaaggatccatatgctaatctcatgctgcagcgggaaaaggattgggtttctaaaatccagatgatgcagttgcagagcactgatccctacctggatgatttctattaccagaattactttgaaaaactggagaaaatgtcagctgctgaagaaatacaaggtgatggtcctaagaaggagcgtaccaagctcatcaccccccaggtggccaaactggagcatgcttataagcctgtgcagtttgagggctctttgggtaagcttactgtctctagtgtgaacaatcctcgaaaaatgattgatgctgttgtgacatctcggagtgaggatgacgaaacaaaagaaaaacaagttcgggacaagagaagaaaaacccttgtcataattgagaaaacctatagtttactccttgatgtggaggattatgaaaggcgctatctcctaagtctagaagaagagcggcctgccctggtggatgaaagaaagcacaaaatctgtagcatgtatgacaacctgaggggcaaattgcctggacaagaaaggccaagcgatgaccactttgtacagatcatgtgtatccgaaaagggaagagaatggttgcccgcattctccctttcctctccacagagcaagcagctgacattcttatggcaactgccaggaacctccctttccttatcaagaaggacgcacaagatgaggtgctgccatgcttactgagtcccttctctcttctcctctatcatcttccttcagtgactgtcaccagccttctgcaacagctaatgaacctacctcaaagtgcaactgcaccagcaccctccaatcctcacctcactgctgtgctccagaacaagtttggcttgtcactgctcctaatccttctaagccgtggagaagacctacagagttcagatcctgctatagaatcaacgcaaaataaccaatggacggaggtgatgttcatggcaacccgagaacttctacagatcccccaagcagccctggctaagccaatctctatacccacaaatctagtgtcccttttttctcgctatgttgaccggcagaaactgaacttgctggagacaaaactacagctggttcaggggatacgataa

SEQ ID NO: 78 - DNA, 2316 bp, synthetic construct
  source 1..2316: mol_type = other DNA
atgttccgctacgagtctttagaggattgtcctctagatgaagatgaagatgcatttcaaggcctgggagaagaagatgaagaaattgatcaattcaatgatgatacatttgggtcaggtgcagttgatgatgattggcaggaagcacatgagcgcctggctgagttggaagagaagctacctgcggcagctgatgagcaaacaggcaatagagagagggatgagatggacttgttgggtgaccatgaggagaatctagcagaaaggctcagtaagatggtgattgaaaatgagctagaagatccagctatcatgagggcagtgcagaccaggccggttttacaacctgaacccagaagtctgaattctagcatctgggatggatctgaagttctgaggcgaatccgtggaccactacttgctcaggtagaaatgcctacggtgtctgtattagaatatgccttgcctcagaggcccccacagggcccagaagatgatcgagacctttctgagcgagctttaccaaggcgttctacatcacctgtcattggaagtcctcctgttagagccgttcctataggcaccccacctaagcagatggccgttcccagcttcaaccaacagattctgtgtccgaagcctgttcatgtacgacctccgatgccaccacgttatcctgctccctatggtgagaggatgtctccaaaccagctctgcagtgtcccgaattcgtcccttctgggccacccttttcctcctagtgttcctcctgtactcagtcccctgcagagagcacagcttcttggaggagcacagctacagcctggtcgtatgtctcccagccagtttgcacgagtccctggatttgttggtagccccctggctgccatgaatcctaagttgctacaagggcgagttggacagatacttcccccagcaccaggcttccgtgccttctttagtgctccaccccctgctacaccccctccccaacagcatcctcctggcccaggacctcacctgcaaaacttaagacctcaggccccaatgtttagaccagacacaactcaccttcatccacagcaccggcgcctgttgcatcagaggcagcagcagagtagaaatcagcatcggaatctcaatggtgctggagacagaggaagtcaccgggccaatcatcaagatcatctccggaaggatccatatgctaatctcatgctgcagcgggaaaaggattgggtttctaaaatccagatgatgcagttgcagagcactgatccctacctggatgatttctattaccagaattactttgaaaaactggagaaaatgtcagctgctgaagaaatacaaggtgatggtcctaagaaggagcgtaccaagctcatcaccccccaggtggccaaactggagcatgcttataagcctgtgcagtttgagggctctttgggtaagcttactgtctctagtgtgaacaatcctcgaaaaatgattgatgctgttgtgacatctcggagtgaggatgacgaaacaaaagaaaaacaagttcgggacaagagaagaaaaacccttgtcataattgagaaaacctatagtttactccttgatgtggaggattatgaaaggcgctatctcctaagtctagaagaagagcggcctgccctggtggatgaaagaaagcacaaaatctgtagcatgtatgacaacctgaggggcaaattgcctggacaagaaaggccaagcgatgaccactttgtacagatcatgtgtatccgaaaagggaagagaatggttgcccgcattctccctttcctctccacagagcaagcagctgacattcttatggcaactgccaggaacctccctttccttatcaagaaggacgcacaagatgaggtgctgccatgcttactgagtcccttctctcttctcctctatcatcttccttcagtgactgtcaccagccttctgcaacagctaatgaacctacctcaaagtgcaactgcaccagcaccctccaatcctcacctcactgctgtgctccagaacaagtttggcttgtcactgctcctaatccttctaagccgtggagaagacctacagagttcagatcctgctatagaatcaacgcaaaataaccaatggacggaggtgatgttcatggcaacccgagaacttctacagatcccccaagcagccctggctaagccaatctctatacccacaaatctagtgtcccttttttctcgctatgttgaccggcagaaactgaacttgctggagacaaaactacagctggttcaggggatacgataa

SEQ ID NO: 79 - DNA, 2058 bp, synthetic construct
  source 1..2058: mol_type = other DNA
atgaagatgaagatgcatttcaaggcctgggagaagaagatgaagaaattgatcaattcaatgatgatacatttgggtcaggtgcagttgcctgaacccagaagtctgaattctagcatctgggatggatctgaagttctgaggcgaatccgtggaccactacttgctcaggaaatgcctacggtgtctgtattagaatatgccttgcctcagaggcccccacagggcccagaagatgatcgagacctttctgagcgagctttaccaaggcgttctacatcacctgtcattggaagtcctcctgttagagccgttcctataggcaccccacctaagcagatggccgttcccagcttcaaccaacagattctgtgtccgaagcctgttcatgtacgacctccgatgccaccacgttatcctgctccctatggtgagaggatgtctccaaaccagctctgcagtgtcccgaattcgtcccttctgggccacccttttcctcctagtgttcctcctgtactcagtcccctgcagagagcacagcttcttggaggagcacagctacagcctggtcgtatgtctcccagccagtttgcacgagtccctggatttgttggtagccccctggctgccatgaatcctaagttgctacaagggcgagttggacagatacttcccccagcaccaggcttccgtgccttctttagtgctccaccccctgctacaccccctccccaacagcatcctcctggcccaggacctcacctgcaaaacttaagacctcaggccccaatgtttagaccagacacaactcaccttcatccacagcaccggcgcctgttgcatcagaggcagcagcagagtagaaatcagcatcggaatctcaatggtgctggagacagaggaagtcaccgggccaatcatcaagatcatctccggaaggatccatatgctaatctcatgctgcagcgggaaaaggattgggtttctaaaatccagatgatgcagttgcagagcactgatccctacctggatgatttctattaccagaattactttgaaaaactggagaaaatgtcagctgctgaagaaatacaaggtgatggtcctaagaaggagcgtaccaagctcatcaccccccaggtggccaaactggagcatgcttataagcctgtgcagtttgagggctctttgggtaagcttactgtctctagtgtgaacaatcctcgaaaaatgattgatgctgttgtgacatctcggagtgaggatgacgaaacaaaagaaaaacaagttcgggacaagagaagaaaaacccttgtcataattgagaaaacctatagtttactccttgatgtggaggattatgaaaggcgctatctcctaagtctagaagaagagcggcctgccctggtggatgaaagaaagcacaaaatctgtagcatgtatgacaacctgaggggcaaattgcctggacaagaaaggccaagcgatgaccactttgtacagatcatgtgtatccgaaaagggaagagaatggttgcccgcattctccctttcctctccacagagcaagcagctgacattcttatggcaactgccaggaacctccctttccttatcaagaaggacgcacaagatgaggtgctgccatgcttactgagtcccttctctcttctcctctatcatcttccttcagtgactgtcaccagccttctgcaacagctaatgaacctacctcaaagtgcaactgcaccagcaccctccaatcctcacctcactgctgtgctccagaacaagtttggcttgtcactgctcctaatccttctaagccgtggagaagacctacagagttcagatcctgctatagaatcaacgcaaaataaccaatggacggaggtgatgttcatggcaacccgagaacttctacagatcccccaagcagccctggctaagccaatctctatacccacaaatctagtgtcccttttttctcgctatgttgaccggcagaaactgaacttgctggagacaaaactacagctggttcaggggatacgataa

SEQ ID NO: 80 - DNA, 2313 bp, synthetic construct
  source 1..2313: mol_type = other DNA
atgttccgctacgagtctttagaggattgtcctctagatgaagatgaagatgcatttcaaggcctgggagaagaagatgaagaaattgatcaattcaatgatgatacatttgggtcaggtgcagttgatgatgattggcaggaagcacatgagcgcctggctgagttggaagagaagctacctgcggcagctgatgagcaaacaggcaatagagagagggatgagatggacttgttgggtgaccatgaggagaatctagcagaaaggctcagtaagatggtgattgaaaatgagctagaagatccagctatcatgagggcagtgcagaccaggccggttttacaacctgaacccagaagtctgaattctagcatctgggatggatctgaagttctgaggcgaatccgtggaccactacttgctcaggaaatgcctacggtgtctgtattagaatatgccttgcctcagaggcccccacagggcccagaagatgatcgagacctttctgagcgagctttaccaaggcgttctacatcacctgtcattggaagtcctcctgttagagccgttcctataggcaccccacctaagcagatggccgttcccagcttcaaccaacagattctgtgtccgaagcctgttcatgtacgacctccgatgccaccacgttatcctgctccctatggtgagaggatgtctccaaaccagctctgcagtgtcccgaattcgtcccttctgggccacccttttcctcctagtgttcctcctgtactcagtcccctgcagagagcacagcttcttggaggagcacagctacagcctggtcgtatgtctcccagccagtttgcacgagtccctggatttgttggtagccccctggctgccatgaatcctaagttgctacaagggcgagttggacagatacttcccccagcaccaggcttccgtgccttctttagtgctccaccccctgctacaccccctccccaacagcatcctcctggcccaggacctcacctgcaaaacttaagacctcaggccccaatgtttagaccagacacaactcaccttcatccacagcaccggcgcctgttgcatcagaggcagcagcagagtagaaatcagcatcggaatctcaatggtgctggagacagaggaagtcaccgggccaatcatcaagatcatctccggaaggatccatatgctaatctcatgctgcagcgggaaaaggattgggtttctaaaatccagatgatgcagttgcagagcactgatccctacctggatgatttctattaccagaattactttgaaaaactggagaaaatgtcagctgctgaagaaatacaaggtgatggtcctaagaaggagcgtaccaagctcatcaccccccaggtggccaaactggagcatgcttataagcctgtgcagtttgagggctctttgggtaagcttactgtctctagtgtgaacaatcctcgaaaaatgattgatgctgttgtgacatctcggagtgaggatgacgaaacaaaagaaaaacaagttcgggacaagagaagaaaaacccttgtcataattgagaaaacctatagtttactccttgatgtggaggattatgaaaggcgctatctcctaagtctagaagaagagcggcctgccctggtggatgaaagaaagcacaaaatctgtagcatgtatgacaacctgaggggcaaattgcctggacaagaaaggccaagcgatgaccactttgtacagatcatgtgtatccgaaaagggaagagaatggttgcccgcattctccctttcctctccacagagcaagcagctgacattcttatggcaactgccaggaacctccctttccttatcaagaaggacgcacaagatgaggtgctgccatgcttactgagtcccttctctcttctcctctatcatcttccttcagtgactgtcaccagccttctgcaacagctaatgaacctacctcaaagtgcaactgcaccagcaccctccaatcctcacctcactgctgtgctccagaacaagtttggcttgtcactgctcctaatccttctaagccgtggagaagacctacagagttcagatcctgctatagaatcaacgcaaaataaccaatggacggaggtgatgttcatggcaacccgagaacttctacagatcccccaagcagccctggctaagccaatctctatacccacaaatctagtgtcccttttttctcgctatgttgaccggcagaaactgaacttgctggagacaaaactacagctggttcaggggatacgataa

SEQ ID NO: 81 - DNA, 2061 bp, Cricetulus griseus; identical to SEQ ID NO: 77
  source 1..2061: mol_type = other DNA

SEQ ID NO: 82 - DNA, 20 bp, synthetic construct
  source 1..20: mol_type = other DNA
agcatgtatgacaacctgag

SEQ ID NO: 83 - DNA, 20 bp, synthetic construct
  source 1..20: mol_type = other DNA
tagggagcaggataacgtgg

SEQ ID NO: 84 - DNA, 20 bp, synthetic construct
  source 1..20: mol_type = other DNA
tgttctggagcacagcagtg

SEQ ID NO: 85 - DNA, 20 bp, synthetic construct
  source 1..20: mol_type = other DNA
ttgaggggagggagtgtagg

SEQ ID NO: 86 - DNA, 53 bp, synthetic construct
  source 1..53: mol_type = other DNA
tcgtcggcagcgtcagatgtgtataagagacagttgaggggagggagtgtagg

SEQ ID NO: 87 - DNA, 25 bp, synthetic construct
  source 1..25: mol_type = other DNA
actaaaggtgatcatgaagttcaca

SEQ ID NO: 88 - DNA, 59 bp, synthetic construct
  source 1..59: mol_type = other DNA
gtctcgtgggctcggagatgtgtataagagacagactaaaggtgatcatgaagttcaca

SEQ ID NO: 89 - DNA, 21 bp, synthetic construct
  source 1..21: mol_type = other DNA
tcctgacctagcctgttctgt

SEQ ID NO: 90 - DNA, 54 bp, synthetic construct
  source 1..54: mol_type = other DNA
tcgtcggcagcgtcagatgtgtataagagacagtcctgacctagcctgttctgt

SEQ ID NO: 91 - DNA, 20 bp, synthetic construct
  source 1..20: mol_type = other DNA
tagtggcaatggaaggggtc

SEQ ID NO: 92 - DNA, 54 bp, synthetic construct
  source 1..54: mol_type = other DNA
gtctcgtgggctcggagatgtgtataagagacagtagtggcaatggaaggggtc

SEQ ID NO: 93 - DNA, 24 bp, synthetic construct
  source 1..24: mol_type = other DNA
acagttggtagagtgtaacgttct

SEQ ID NO: 94 - DNA, 57 bp, synthetic construct
  source 1..57: mol_type = other DNA
tcgtcggcagcgtcagatgtgtataagagacagacagttggtagagtgtaacgttct

SEQ ID NO: 95 - DNA, 24 bp, synthetic construct
  source 1..24: mol_type = other DNA
tcctaagtcaatatagatgccaca

SEQ ID NO: 96 - DNA, 58 bp, synthetic construct
  source 1..58: mol_type = other DNA
gtctcgtgggctcggagatgtgtataagagacagtcctaagtcaatatagatgccaca

SEQ ID NO: 97 - protein, 770 aa, Cricetulus griseus; identical to SEQ ID NO: 14

SEQ ID NO: 98 - protein, 771 aa, Cricetulus griseus
MFRYESLEDCPLDEDEDAFQGLGEEDEEIDQFNDDTFGSGAVDDDWQEAHERLAELEEKLPAAADEQTGNRERDEMDLLGDHEENLAERLSKMVIENELEDPAIMRAVQTRPVLQPEPRSLNSSIWDGSEVLRRIRGPLLAQVEMPTVSVLEYALPQRPPQGPEDDRDLSERALPRRSTSPVIGSPPVRAVPIGTPPKQMAVPSFNQQILCPKPVHVRPPMPPRYPAPYGERMSPNQLCSVPNSSLLGHPFPPSVPPVLSPLQRAQLLGGAQLQPGRMSPSQFARVPGFVGSPLAAMNPKLLQGRVGQILPPAPGFRAFFSAPPPATPPPQQHPPGPGPHLQNLRPQAPMFRPDTTHLHPQHRRLLHQRQQQSRNQHRNLNGAGDRGSHRANHQDHLRKDPYANLMLQREKDWVSKIQMMQLQSTDPYLDDFYYQNYFEKLEKMSAAEEIQGDGPKKERTKLITPQVAKLEHAYKPVQFEGSLGKLTVSSVNNPRKMIDAVVTSRSEDDETKEKQVRDKRRKTLVIIEKTYSLLLDVEDYERRYLLSLEEERPALVDERKHKICSMYDNLRGKLPGQERPSDDHFVQIMCIRKGKRMVARILPFLSTEQAADILMATARNLPFLIKKDAQDEVLPCLLSPFSLLLYHLPSVTVTSLLQQLMNLPQSATAPAPSNPHLTAVLQNKFGLSLLLILLSRGEDLQSSDPAIESTQNNQWTEVMFMATRELLQIPQAALAKPISIPTNLVSLFSRYVDRQKLNLLETKLQLVQGIR

SEQ ID NO: 99 - protein, 770 aa, Cricetulus griseus; identical to SEQ ID NO: 14

SEQ ID NO: 100 - protein, 686 aa, Cricetulus griseus; identical to SEQ ID NO: 15

SEQ ID NO: 101 - protein, 685 aa, Cricetulus griseus; identical to SEQ ID NO: 16

(19) United States
(12) Patent Application Publication
(10) Pub. No.: US 2026/0218215 A1
(43) Pub. Date: Jul. 30, 2026

(54) HOST CELLS WITH PAT1 KNOCKOUT FOR INCREASED SPECIFIC PROTEIN PRODUCTIVITY

(71) Applicant: Lonza Ltd, Visp (CH)

(72) Inventors: Jennifer STAUDACHER, Vienna (AU); Brigitte GASSER, Vienna (AU); Marc FEARY, Slough, Berkshire (GB); Louise JONES, Slough, Berkshire (GB)

(21) Appl. No.: 19/141,870

(22) PCT Filed: Dec. 20, 2023
(86) PCT No.: PCT/EP2023/087067

§ 371 Date: Jun. 20, 2025

(30) Foreign Application Priority Data
Dec. 20, 2022 (EP) 22215068.2

(51) Int. Cl. C12N 15/81; C07K 14/765; C07K 16/00; C12N 9/22; C12N 15/11; C12N 15/90

(52) U.S. Cl. CPC C12N 15/815; C07K 14/765; C07K 16/00; C12N 9/226; C12N 15/11; C12N 15/907; C07K 2317/14; C07K 2317/35; C07K 2317/569; C12N 2310/20

(57) ABSTRACT

A eukaryotic host cell which is engineered to introduce a recombinant expression cassette expressing a gene of interest (GOI) to produce a protein of interest (POI), and by a genetic modification to control expression of a Pat1 protein.

### FIELD OF THE INVENTION

The invention refers to eukaryotic host cells engineered to improve the yield of a protein of interest by improving the translational machinery of the cells.

### BACKGROUND OF THE INVENTION

Proteins produced in engineered host cells or transfected cell culture have become increasingly important as diagnostic and therapeutic agents. For this purpose, cells are engineered and/or selected to produce unusually high levels of a recombinant or heterologous protein of interest.

Successful production of proteins of interest (POI) has been accomplished with eukaryotic host cells in cell culture. Eukaryotic host cells, in particular mammalian host cells, yeasts or filamentous fungi, or bacteria are commonly used as production hosts for biopharmaceutical proteins as well as for bulk chemicals. The most prominent examples for production hosts are methylotrophic yeasts such as Pichia pastoris, which is well reputed for efficient secretion of heterologous proteins. In 2005, P. pastoris has been reclassified into a new genus, Komagataella, and split into three species, K. pastoris, K. phaffii, and K. pseudopastoris. Strains commonly used for biotechnological applications belong to K. pastoris and K. phaffii. The strains GS115, X-33, CBS2612, and CBS7435 are K. phaffii, while the strain DSMZ70382 is classified into the type species, K. pastoris, which is the reference strain for all the available P. pastoris strains (Kurtzman 2009, J Ind Microbiol Biotechnol. 36(11):1435-8). Mattanovich et al. (Microbial Cell Factories 2009, 8:29 doi:10.1186/1475-2859-8-29) describe the genome sequencing of the type strain DSMZ70382 of K. pastoris, and analyzed its secretome and sugar transporters.

The yield of protein production in yeast host cells can e.g., be modulated by either the selection of a strong promoter, the targeted engineering of components of the secretory and the translational pathway, the overexpression or knockout of transcription factors, and by bioprocess engineering approaches.

Rebnegger et al. (Biotechnol. J. 2014, 9, 511-525) describe the regulation of protein synthesis and secretion, mating, and stress response by growth rate in Pichia pastoris.

Protein synthesis includes transcription of the gene, translation of the mRNA, folding of the nascent polypeptide and optional post-translational modifications. Yeast cells are known to downregulate this general protein synthesis pathway (Rebnegger et al. 2014. Biotechnol J. 2014 April; 9(4):511-25. doi: 10.1002/biot.201300334.) when growing at reduced rates. In changing environmental conditions, such as the industrially relevant reduction of carbon source, cells appear to reduce translation by inactivating as well as degrading mRNAs and by direct inhibition of the translation initiation process (Sachdev et al. 2019. Elife. 2019 Jan. 16; 8:e41415. doi: 10.7554/eLife.41415.).

Translation initiation is a significant bottleneck for protein synthesis and overexpression of related factors increases global synthesis. This, in turn, has beneficial effects on recombinant protein production (Staudacher et al. (Metabolic Engineering 2022, 70, 181-195; WO2022069613A1).

Due to the tight connection between mRNA decay and mRNA translation, both processes are in constant competition with each other, which means mRNA decay may further limit recombinant protein production. In eukaryotes, mRNA storage/decay happens in so-called processing bodies (P-bodies). They have been studied extensively for their components in Saccharomyces cerevisiae.

Marnef et al (Biochem Soc Trans. 2010; 38(6):1602-7) describe Pat1 proteins as involved in several key post-transcriptional mechanisms of gene expression control. In yeast, Pat1p interacts with translating mRNPs (messenger ribonucleoproteins), and is responsible for translational repression and decapping activation, ultimately leading to mRNP degradation. Drosophila HPat and human Pat1b (PatL1) proteins also have conserved their roles.

Coller and Parker (Cell 2005, 122(6), 875-886) describe the general translational repression by activators of eukaryotic mRNA decapping and identified decapping activators Dhh1p or Pat1p as translational repressors and facilitators of P-body formation.

Sachdev et al. (eLife 2019, 8:e41415, 1-27) describe the DEAD-box ATPase Dhh1 as a key regulator of P-body dynamics, and a role for Pat1 in promoting the multimerization of the Dhh1 on mRNA.

Influencing general mRNA decay has been attempted in Escherichia coli before, but as the bacterial mechanism is completely different, the approaches are not comparable (Roux et al. 2022. Biotechnol Adv. 2022; 54:107805. doi: 10.1016/j.biotechadv.2021.107805).

Nissan et al. (Molecular Cell 2010, 39(5):733-783) disclose decapping activators in Saccharomyces cerevisiae that act by multiple mechanisms. Pat1 has been identified as critical in mRNA decay by first inhibiting translation initiation, then serving as a scaffold to recruit components of the decapping complex, and finally activating Dcp2.

Pilkington et al. (Molecular and Cellular Biology 2008, 28(4):1298-1312) disclose that Pat1 in S. cerevisiae contains distinct functional domains that promote P-body assembly and activation of decapping.

WO2020/144313 discloses a recombinant host cell that is engineered to reduce expression of the host cell's gene encoding FLO8, and expressing a gene of interest from a heterologous expression cassette in such host cell under the control of an expression cassette promoter that is repressible by a carbon source that is not methanol.

Wyers et al. (Molecular and Cellular Biology 2000, 20(10):3538-3549) disclose that deletion of the PAT1 gene affects translation initiation and suppresses a PAB1 gene deletion in yeast.

He Feng et al. (eLIFE 2018, e34409) disclose that general decapping activators target different subsets of inefficiently translated mRNAs.

Staudacher et al. (Metabolic Engineering 2022, 70:181-195) disclose that increasing global translation activity would lead to increased productivity of recombinant secreted proteins in Pichia pastoris.

Coller et al. (Cell 2005, 122(6):875-886) disclose general translational repression by activators of mRNA decapping.

There is a need for new strategies for cell engineering that lead to enhanced protein production, which do not target the transcription machinery or the secretory pathway.

### SUMMARY OF THE INVENTION

This summary is provided to introduce a selection of concepts in a simplified form that are further described below in the detailed description. This summary is not intended to identify key or essential features of the claimed subject matter, nor is it intended to be used to limit the scope of the claimed subject matter. Other features, details, utilities, and advantages of the claimed subject matter will be apparent from the following written detailed description, including those aspects illustrated in the accompanying drawings and defined in the appended claims.

It is the objective of the invention to provide new eukaryotic host cells engineered for improved protein production. It is a particular object to provide improved host cells and production methods employing carbon source-limiting conditions in the production phase.

The objective is solved by the claimed subject matter, and as further described herein.

The invention provides for a eukaryotic host cell which is engineered to introduce a recombinant expression cassette expressing a gene of interest (GOI) to produce a protein of interest (POI), and by a genetic modification to control expression of a Pat1 protein.

The eukaryotic host cell as described herein can be used as production host cells (also referred to as “producer cells”) that are suitably used in a production process at industrial scale. Specific examples refer to yeast and mammalian host cells, such as P. pastoris, CHO or human host cells.

Preferably the eukaryotic host cell is selected from yeast such as Pichia or Pichia Pastoris (e.g., selected from K. phaffii, K. pastoris, or K. pseudopastoris), CHO cells (Cricetulu griseus), human (Homo sapiens), or Drosophila, in particular, D. melanogaster.

The eukaryotic host cell is an improved host cell which can be used in a method of producing a POI at a higher yield and/or titer (in particular as compared to the host cell without such genetic modification to control expression of the host cell's Pat1 protein) when expressing and optionally secreting the POI under carbon source-limiting conditions in the production phase e.g., in the production phase of a fed-batch process.

Specifically, the genetic modification controls, in particular reduces or increases expression of the Pat1 protein.

Specifically, the genetic modification controls, in particular reduces or increases expression of the endogenous Pat1 protein, compared to a cell of the same type in which the level of Pat1 protein has not been controlled, or compared to an endogenous expression thereof.

Specifically, the genetic modification controls, in particular reduces or increases expression of the Pat1 protein, compared to its expression without such genetic engineering e.g., in a parental cell line, which is herein understood as the same cell line or the same type of cell line, but without the respective genetic modification to control the Pat1 expression.

Specifically, the expression of the Pat1 protein is controlled, in particular reduced, by said genetic modification.

Specifically. the genetic modification may comprise one or more genetic modifications, in particular one or more types of genetic modifications.

Specifically, said Pat1 protein expression that is controlled in the host cell refers to an endogenous expression of Pat1 by the host cell, which is herein understood as an expression of Pat1 from the host cell's genome, in particular from the host cell's endogenous gene i.e., the gene that is endogenous of the wild-type host cell. By controlling the host cell's endogenous expression of Pat1, the level or amount of Pat1 that is expressed and/or secreted by the host cell is controlled.

Specifically, the level or amount of the Pat1 protein is controlled by said genetic modification, and controlling the level or amount of the Pat1 protein increases the POI yield compared to the host cell without said genetic modification to control expression of the Pat1 protein.

Specifically, the host cell has an increased POI yield and/or titer compared to a cell of the same type in which the level of Pat1 protein has not been controlled.

Specifically, the POI yield and/or titer is increased in said host cell by said genetic modification to control expression of the Pat1 protein, as compared to the respective strain without such genetic modification.

According to a specific aspect, the host cell comprises an increased POI yield and/or titer.

Specifically, the POI yield and/or titer of the host cell is at least 1.2-fold or at least 1.3-fold increased.

Specifically, the POI yield and/or titer is increased by any one of 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold.

Specifically, the POI yield and/or titer is increased by at least 1.2-fold-12-fold, or 1.2-fold-10-fold, or 1.2-fold-8-fold, or 1.2-fold-6-fold, or 1.3-fold-12-fold, or 1.3-fold-10-fold, or 1.3-fold-8-fold, or 1.3-fold-6-fold.

According to a specific aspect, the host cell comprises an increased specific productivity (μg/g cell dry mass (CDM)) per hour and/or an increased volumetric productivity (μg/L per hour) for said POI, compared to a cell of the same type in which the level of Pat1 protein has not been controlled.

Specifically, either one or both of the specific productivity and the volumetric productivity of the host cell is increased in said host cell, compared to a cell of the same type in which the level of Pat1 protein has not been controlled. Specifically, either one or both of the specific productivity and the volumetric productivity of the host cell is increased in said host cell by said genetic modification to control expression of the Pat1 protein as compared to the respective strain without such genetic modification.

Specifically, the host cell comprises an at least 1.1-fold or at least 1.2-fold increased specific productivity (μg/g cell dry mass (CDM), in particular yeast dry mass (YDM) for yeast cells) per hour and/or volumetric productivity (μg/L per hour) for said POI, compared to the host cell without said genetic modification to control expression of the Pat1 protein. Specifically, the specific productivity and/or the volumetric productivity of the host cell is at least 1.2-fold or at least 1.3-fold increased.

Specifically, the specific productivity and/or the volumetric productivity is increased by at least any one of 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold.

Specifically, the specific productivity and/or the volumetric productivity is increased by 1.2-fold-12-fold, or 1.2-fold-10-fold, or 1.2-fold-8-fold, or 1.2-fold-6-fold, or 1.3-fold-12-fold, or 1.3-fold-10-fold, or 1.3-fold-8-fold, or 1.3-fold-6-fold.

According to a specific aspect, said genetic modification reduces the level or amount of Pat1 protein that is expressed by the host cell. Specifically, said genetic modification reduces the host cell's endogenous expression of Pat1.

Specifically, the expression of the Pat1 protein is reduced by said genetic modification. Specifically, said genetic modification comprises a disruption, substitution, deletion or knockout of (i) an endogenous polynucleotide encoding the Pat1 protein, or a part thereof; or (ii) an expression control sequence that controls expression of said polynucleotide encoding the Pat1 protein, preferably wherein the polynucleotide encoding the Pat1 protein is endogenous to the host cell.

Preferably, said genetic modification comprises a knock-out of the host cell's gene that encodes the Pat1 protein. Specifically, the endogenous gene encoding said Pat1 protein is knocked-out.

According to a specific aspect, said genetic modification comprises knocking out a copy of a gene encoding the Pat1 protein from the genome of the host cell, or knocking out a copy of a regulatory nucleic acid sequence operably linked to the gene encoding the Pat1 protein.

Specifically, said genetic modification comprises knocking out all copies of a gene encoding the Pat1 protein from the genome of the host cell.

Specifically, the host cell does not comprise a functional copy of a gene encoding the Pat1 protein and/or a functional copy of a regulatory nucleic acid sequence operably linked to the gene encoding the Pat1 protein.

Specifically, the Pat1 is a protein that is a decapping activator and translational repressor, a topoisomerase II-associated protein, and/or a mRNA turnover protein 1.

The term “Pat1” or “Pat1 protein” shall specifically include:
(i) a protein comprising the amino acid sequence identified as SEQ ID NO:1 or SEQ ID NO:3 (which originate from K. phaffii and K. pastoris, respectively), or any one of SEQ ID NO:13 to 17, or SEQ ID NO:97-101, which originates from a CHO cell,
(ii) an amino acid sequence which has a certain homology to SEQ ID NO:1, SEQ ID NO:3, or any one of SEQ ID NO:13 to 17, or SEQ ID NO:97-101, and/or
(iii) a Pat1 ortholog that is endogenously expressed by a cell which is selected for engineering a production host cell line, in particular wherein the cell endogenously expresses a wild-type Pat1 protein, such as a wild-type cell.

According to a specific aspect, the term “Pat1 homologue” shall include a Pat1 protein comprising or consisting of an amino acid sequence that is homologous or orthologous to any one of SEQ ID NO:1, SEQ ID NO:3, or any one of SEQ ID NO:13 to 17, or SEQ ID NO:97-101.

If the host cell is of a species K. phaffii, in particular a K. phaffii strain CBS7435 or CBS2612, or K. pastoris, the Pat1 protein that is endogenous to the host cell is understood to comprise or consist of SEQ ID NO:1 and SEQ ID NO:3, respectively, and expression of such Pat1 protein in the host cell can be controlled upon engineering the host cell for the purpose described herein.

It is understood that there can be a homologous sequence comprising a certain sequence identity to SEQ ID NO:1 in a host cell of the species K. phaffii because of using strains other than CBS7435 or CBS2612, and/or PAT1 mutations.

Likewise, there can be a homologous sequence comprising a certain sequence identity to SEQ ID NO:3 in a host cell of the species K. pastoris because of using a specific strain which comprise Pat1 that comprises one or more differences (e.g., point mutations) in the Pat1 sequence and/or PAT1 mutations Expression of such homologue in the host cell can be controlled upon engineering the host cell for the purpose described herein.

If the host cell is of a K. phaffii strain other than strains CBS7435 or CBS2612, or of a species different from K. phaffii, the Pat1 protein that is endogenous to the host cell is also understood as an ortholog to SEQ ID NO:1, which may comprise a certain sequence identity to SEQ ID NO:1, and expression of such ortholog in the host cell can be controlled upon engineering the host cell for the purpose described herein.

If the host cell is of a species Cricetulu griseus, in particular a CHO cell line, the Pat1 protein that is endogenous to the host cell is understood to comprise or consist of any one of SEQ ID NO:13 to 17, or SEQ ID NO:97-101, and expression of such Pat1 protein in the host cell can be controlled upon engineering the host cell for the purpose described herein.

It is understood that there can be a homologous sequence comprising a certain sequence identity to any one of SEQ ID NO:13 to 17, or SEQ ID NO:97-101 in a host cell of the species Cricetulu griseus because of using strains other than a CHO cell lines such as described herein, and/or PAT1 mutations. Expression of such homologue in the host cell can be controlled upon engineering the host cell for the purpose described herein.

If the host cell is of a Cricetulu griseus strain other than CHO strains described herein, or of a species different from Cricetulu griseus, the Pat1 protein that is endogenous to the host cell is also understood as an ortholog to any one of SEQ ID NO:13 to 17, or SEQ ID NO:97-101, which may comprise a certain sequence identity to the respective SEQ ID NO:13 to 17, or SEQ ID NO:97-101, and expression of such ortholog in the host cell can be controlled upon engineering the host cell for the purpose described herein.

Specifically, the Pat1 protein naturally-occurring in a wild-type host cell is understood to be a functional Pat1 protein, which characterized by the function as mRNA decapping factor or mRNA decapping activator.

A Pat1 homologue sequence that is endogenously expressed in a host cell is understood to be a functional Pat1 protein which is characterized by about same qualitative function as mRNA decapping factor or mRNA decapping activator, compared to the Pat1 protein of SEQ ID NO:1 in a wild-type P. pastoris, in particular K. pastoris or K. phaffii or Komagataella pseudopastoris, or compared to the Pat 1 protein of any one of SEQ ID NO:13 to 17, or SEQ ID NO:97-101 in a wild-type CHO cell line, though its quantitative activity can be different when compared to the respective Pat1 protein of SEQ ID NO:1 in a wild-type P. pastoris, or the respective SEQ ID NO:13 to 17, or SEQ ID NO:97-101 in a wild-type CHO cell line.

Specifically, a test for determining which protein that originates from a host cell and comprises a certain degree of sequence identity to any one of the Pat1 protein sequences as disclosed herein (“a candidate Pat1 protein”) is a Pat1 homologue or a functional Pat1 protein, can be performed as follows. The candidate Pat1 protein can be determined to be a Pat1 homologue if functional upon adding the candidate Pat1 protein to a culture of said host cell strain (from which the candidate Pat1 protein originates) that is engineered to knockout the endogenous Pat1 encoding gene (e.g., a pat1 knockout strain), or upon incorporating a gene encoding the candidate Pat1 protein in the knockout host cell's genome to express the candidate Pat1 protein in a cell culture of said host cell, whereby functional replacement of the deleted Pat1 protein in the knockout strain determines functionality of the candidate Pat1 protein.

Specifically, a Pat1 protein that is endogenously expressed by a host cell is understood as being endogenous to a respective wild-type host cell. Specifically, the Pat1 protein is encoded by a PAT1 gene that is endogenous to the respective wild-type host cell.

The Pat1 protein is particularly understood to be endogenous to the host cell that is used as recombinant host cell producing the POI as further described herein.

Specifically, the host cell endogenous polynucleotide is endogenous to the wild-type host cell.

Specifically, the Pat1 protein can be an ortholog of the Pat1 protein comprising or consisting of SEQ ID NO:1, SEQ ID NO:3, or any one of SEQ ID NO:13 to 17, or SEQ ID NO:97-101, which ortholog is endogenous to the host cell species.

Specifically, the Pat1 protein is Pat1 protein comprising SEQ ID NO:1, SEQ ID NO:3, or any one of SEQ ID NO:13 to 17, or SEQ ID NO:97-101, or a Pat1 ortholog to any one of the foregoing, preferably wherein the Pat1 is endogenous to the wild-type host cell, in particular wherein the Pat1 protein is encoded by the host cell endogenous polynucleotide without said genetic modification to control expression of the Pat1 protein.

Specifically, the Pat1 protein originates from the host cell.

Specifically, the Pat1 protein is of P. pastoris origin, in particular of K. pastoris or K. phaffii origin, if the host cell is of the respective P. pastoris species. Specifically, the Pat1 protein can be of P. pastoris origin other than K. phaffii.

Specifically, the Pat1 protein can be of a species origin other than P. pastoris e.g., another eukaryotic cell, such as yeast or a filamentous fungal cell, preferably yeast of the Komagataella or Pichia genus, or Saccharomyces genus or any methylotrophic yeast. Yet, the host cell can be a eukaryotic cell other than yeast or filamentous fungi, such as an animal cell, a vertebrate cell, a mammalian cell, a human cell, a plant cell, a bacterial cell, a nematodal cell, an invertebrate cell such as an insect cell, a mollusk cell, or a stem cell, and the respective Pat1 protein is endogenous to the respective host cell, but its expression is controlled, reduced e.g., eliminated or abolished, by engineering the host cell as described herein.

Specifically, the Pat1 protein is of Cricetulu griseus origin, in particular of a CHO cell line such as described herein, if the host cell is of the respective Cricetulu griseus species. Specifically, the Pat1 protein can be of Cricetulu griseus origin other than a CHO cell as described herein.

Specifically, the Pat1 protein can be of a species origin other than P. pastoris or Cricetulu griseus e.g., another eukaryotic cell, such as yeast or a filamentous fungal cell, preferably yeast of the Komagataella or Pichia genus, or Saccharomyces genus or any methylotrophic yeast. Yet, the host cell can be a eukaryotic cell other than yeast or filamentous fungi, such as an animal cell, a vertebrate cell, a mammalian cell, a human cell, a plant cell, a bacterial cell, a nematodal cell, an invertebrate cell such as an insect cell, a mollusk cell, or a stem cell, and the respective Pat1 protein is endogenous to the respective host cell, but its expression is controlled, reduced e.g., eliminated or abolished, by engineering the host cell as described herein.

Specifically, the Pat1 protein is Pat1 protein comprising SEQ ID NO:1, SEQ ID NO:3, or any one of SEQ ID NO:13 to 17, or SEQ ID NO:97-101, or a Pat1 ortholog to any one of the foregoing, preferably wherein the Pat1 is endogenous to the wild-type host cell, in particular wherein the Pat1 is encoded by the host cell endogenous polynucleotide without said genetic modification to control expression of the Pat1 protein.

Specifically, the host cell is a production host cell suitable for use in a recombinant host cell culture to produce the POI. The production host cell can be used for POI production at an industrial scale.

According to a specific aspect, the eukaryotic host cell is a yeast or mammalian host cell. Specifically, the host cell is a methylotrophic yeast, or a vertebrate host cells, such as derived from a Chinese hamster ovary cell (CHO) cell, mouse, rat, Syrian hamster, monkey, ape, dog, horse, ferret, or cat cell.

Specifically, the host cell is selected from the group consisting of any one of Komagataella phaffii, Komagataella pastoris, Komagataella pseudopastoris, Saccharomyces cerevisiae, Yarrowia lipolytica, Ogataea polymorpha, Schizosaccharomyces pombe, Trichoderma reesei, Kluyveromyces lactis, Aspergillus niger, Homo sapiens, or Cricetulu griseus host cells.

Specifically, the host cell is a mammalian cell, in particular a CHO cell, such as e.g., a CHO cell which is any of CHOK1, CHOK1SV, Potelligent CHOK1SV, CHO GS knockout, CHOK1SV GS-KO, CHOS, CHO DG44, CHO DXB11, CHOZN, or a CHO-derived cell.

Specific preferred examples of a Pat1 protein are selected from a Pat1 protein originating from a CHO cell, wherein the Pat1 protein is e.g., encoded by a nucleic acid which comprises or consists of at least 60%, or at least 70%, or at least 80%, or at least 90%, or at least 95%, or at least 96%, or at least 97%, or at least 98%, or at least 99%, or 100% sequence identity to any one of SEQ ID NO:77-80.

Specifically, the host cell is a host cell line, such as an immortalized host cell line.

Specifically,
a) the host cell is a Komagataella phaffii host cell, and the Pat1 protein comprises or consists of at least 90%, or at least 95%, or at least 96%, or at least 97%, or at least 98%, or at least 99%, or 100% sequence identity to SEQ ID NO:1; or
b) the host cell is a Komagataella pastoris host cell, and the Pat1 protein comprises or consists of at least 90%, or at least 95%, or at least 96%, or at least 97%, or at least 98%, or at least 99%, or 100% sequence identity to SEQ ID NO:3; or
c) the host cell is a Saccharomyces cerevisiae host cell, and the Pat1 protein comprises or consists of at least 90%, or at least 95%, or at least 96%, or at least 97%, or at least 98%, or at least 99%, or 100% sequence identity to SEQ ID NO:4; or
d) the host cell is a Yarrowia lipolytica host cell, and the Pat1 protein comprises or consists of at least 90%, or at least 95%, or at least 96%, or at least 97%, or at least 98%, or at least 99%, or 100% sequence identity to SEQ ID NO:5; or
e) the host cell is an Ogataea polymorpha host cell, and the Pat1 protein comprises or consists of at least 90%, or at least 95%, or at least 96%, or at least 97%, or at least 98%, or at least 99%, or 100% sequence identity to SEQ ID NO:6; or
f) the host cell is a Schizosaccharomyces pombe host cell, and the Pat1 protein comprises or consists of at least 90%, or at least 95%, or at least 96%, or at least 97%, or at least 98%, or at least 99%, or 100% sequence identity to SEQ ID NO:7; or
g) the host cell is a Trichoderma reesei host cell, and the Pat1 protein comprises or consists of at least 90%, or at least 95%, or at least 96%, or at least 97%, or at least 98%, or at least 99%, or 100% sequence identity to SEQ ID NO:8; or
h) the host cell is a Kluyveromyces lactis host cell, and the Pat1 protein comprises or consists of at least 90%, or at least 95%, or at least 96%, or at least 97%, or at least 98%, or at least 99%, or 100% sequence identity to SEQ ID NO:9; or
i) the host cell is an Aspergillus niger host cell, and the Pat1 protein comprises or consists of at least 90%, or at least 95%, or at least 96%, or at least 97%, or at least 98%, or at least 99%, or 100% sequence identity to SEQ ID NO:10.
j) the host cell is a Homo sapiens host cell, and the Pat1 protein comprises or consists of at least 90%, or at least 95%, or at least 96%, or at least 97%, or at least 98%, or at least 99%, or 100% sequence identity to SEQ ID NO:11 or 12; or
k) the host cell is a Cricetulu griseus host cell, and the Pat1 protein comprises or consists of at least 90%, or at least 95%, or at least 96%, or at least 97%, or at least 98%, or at least 99%, or 100% sequence identity to any one of SEQ ID NO:13 to 17, or any one of SEQ ID NO:97-101.

Specifically, the Pat1 protein is of Komagataella phaffiiorigin, which comprises or consists of SEQ ID NO:1. Specifically, such Pat1 protein is encoded by a PAT1 gene that is endogenous to the host cell, wherein the host cell is Komagataella phaffii.

Specifically, the Pat1 protein is encoded by the nucleotide sequence identified as SEQ ID NO:2, or a nucleic acid which comprises at least 60%, or at least 70%, or at least 80%, or at least 90%, or at least 95%, or at least 96%, or at least 97%, or at least 98%, or at least 99%, or 100% sequence identity to SEQ ID NO:2.

The degree of sequence identity of Pat1 proteins in various host cell species is further provided herein in the table “Sequence identity of the exemplary Pat1 proteins to Pat1 of K. phaffii (SEQ ID NO:1)”.

Specifically, the Pat1 protein is of Komagataella origin, which comprises at least 90%, 91% or 92% sequence identity to SEQ ID NO:1 or SEQ ID NO:3. Specifically, the Pat1 protein is of Komagataella pastoris origin, which comprises or consists of SEQ ID NO:3. Specifically, such Pat1 protein is encoded by a PAT1 gene that is endogenous to the host cell, wherein the host cell is of the Komagataella pastoris species.

Specifically, the Pat1 protein is of Saccharomyces origin, which comprises at least 35%, 36%, 37%, 38% or 39% sequence identity to SEQ ID NO:1. Specifically, the Pat1 protein is of S. cerevisiae origin, which comprises or consists of SEQ ID NO:4. Specifically, such Pat1 protein is encoded by a gene that is endogenous to the host cell, wherein the host cell is of the S. cerevisiae species.

Specifically, the Pat1 protein is of Yarrowia origin, which comprises at least 30%, 31%, 32%, 33% or 34% sequence identity to SEQ ID NO:1. Specifically, the Pat1 protein is of Yarrowia lipolytica origin, which comprises or consists of SEQ ID NO:5. Specifically, such Pat1 protein is encoded by a gene that is endogenous to the host cell, wherein the host cell is of the Yarrowia lipolytica species.

Specifically, the Pat1 protein is of Ogataea origin, which comprises at least 45%, 46%, 47%, 48% or 49% sequence identity to SEQ ID NO:1. Specifically, the Pat1 protein is of Ogataea polymorpha origin, which comprises or consists of SEQ ID NO:6.

Specifically, such Pat1 protein is encoded by a gene that is endogenous to the host cell, wherein the host cell is of the Ogataea polymorpha species.

Specifically, the Pat1 protein is of Schizosaccharomyces origin, which comprises at least 23%, 24%, 25% or 26% sequence identity to SEQ ID NO:1. Specifically, the Pat1 protein is of Schizosaccharomyces pombe origin, which comprises or consists of SEQ ID NO:7. Specifically, such Pat1 protein is encoded by a gene that is endogenous to the host cell, wherein the host cell is of the Schizosaccharomyces pombe species.

Specifically, the Pat1 protein is of Trichoderma origin, which comprises at least 25%, 26% or 27% sequence identity to SEQ ID NO:1. Specifically, the Pat1 protein is of Trichoderma reesei origin, which comprises or consists of SEQ ID NO:8. Specifically, such Pat1 protein is encoded by a gene that is endogenous to the host cell, wherein the host cell is of the Trichoderma reesei species.

Specifically, the Pat1 protein is of Kluyveromyces origin, which comprises at least 38%, 39%, 40% or 41% sequence identity to SEQ ID NO:1. Specifically, the Pat1 protein is of Kluyveromyces lactis origin, which comprises or consists of SEQ ID NO:9.

Specifically, such Pat1 protein is encoded by a gene that is endogenous to the host cell, wherein the host cell is of the Kluyveromyces lactis species.

Specifically, the Pat1 protein is of Aspergillus origin, which comprises at least 25%, 26% or 27% sequence identity to SEQ ID NO:1. Specifically, the Pat1 protein is of Aspergillus nigerorigin, which comprises or consists of SEQ ID NO:10. Specifically, such Pat1 protein is encoded by a gene that is endogenous to the host cell, wherein the host cell is of the Aspergillus niger species.

Specifically, the Pat1 protein is of Homo sapiens origin, which comprises at least 20%, 21% or 22% sequence identity to SEQ ID NO:1. Specifically, the Pat1 protein is of Homo sapiens origin, which comprises or consists of any one of SEQ ID NO:11 or 12.

Specifically, such Pat1 protein is encoded by a gene that is endogenous to the host cell, wherein the host cell is of the Homo sapiens species.

Specifically, the Pat1 protein is of Cricetulu origin, which comprises at least 20%, 21%, 22% or 23% sequence identity to SEQ ID NO:1. Specifically, the Pat1 protein is of Cricetulu griseus origin, which comprises or consists of any one of SEQ ID NO:13 to 17, or any one of SEQ ID NO:97-101. Specifically, such Pat1 protein is encoded by a gene that is endogenous to the host cell, wherein the host cell is of the Cricetulu griseus species.

A specific Pat1 protein is of a CHO cell line, such as comprising or consisting of any one of SEQ ID NO:13 to 17, or any one of SEQ ID NO:97-101. A specific nucleic acid encoding a Pat1 protein of CHO cells comprises or consists of at least 30%, or at least 40%, or at least 50%, or at least 60%, or at least 70%, or at least 80%, or at least 90%, or at least 95%, or at least 96%, or at least 97%, or at least 98%, or at least 99%, or 100% sequence identity to any one of SEQ ID NO:77-80.

Specifically, the Pat1 homologue comprises or consists of an amino acid sequence which has at least any one of 25%, 30%, or 35% sequence identity to SEQ ID NO:1 e.g., at least any one of 40%, 45%, 50%, 55%, 60%, 65%, 70%, 75%, 80%, 85%, 90%, or 95% sequence identity, or is 100% identical to SEQ ID NO:1. Specifically, sequence identity is determined as further disclosed herein, for example when comparing the full-length sequence.

Specifically, the Pat1 homologue is endogenous to or originating from a Pichia species or endogenous or originating from any other yeast, fungi, mammalian, human, insect, algae or plant host cell, and has at least 25% sequence identity SEQ ID NO: 1, in specific cases at least any one of 30%, 35%, 40%, 45%, 50%, 55%, 60, 65%, 70%, 75%, 80%, 85%, 90%, 91%, 92%, 93%, 94%, 95%, 96%, 97%, 98%, 99% or 100% identical to SEQ ID NO: 1.

According to a specific aspect, the endogenously expressed Pat1 protein comprises or consists of at least any one of 90%, 91%, 92%, 93%, 94%, 95%, 96%, 97%, 98%, 99% or 100% sequence identity to any one of SEQ ID NO:1, or SEQ ID NO:3-17.

According to a specific aspect, the host cell endogenous expression of the Pat1 protein is controlled by one or more genetic modifications of the host cell, such as to reduce expression of said Pat1 protein, in particular to reduce the endogenous Pat1 expression. Specifically, Pat1 expression is reduced by the genetic modification as compared to an endogenous expression thereof.

Specifically, expression is controlled or reduced by one or more genetic modifications comprising a disruption, substitution, deletion or knockout of (i) an endogenous polynucleotide encoding the Pat1 protein, or a part thereof; or (ii) an expression control sequence that controls or reduces expression of said polynucleotide encoding the Pat1 protein, preferably wherein the polynucleotide encoding the Pat1 protein is endogenous to the host cell.

According to a specific aspect, said one or more genetic modifications are of one or more endogenous polynucleotides of the host cell, such as coding polynucleotides, including e.g., said polynucleotide (or gene) encoding the Pat1 protein.

According to a specific aspect, said one or more genetic modifications are of an expression control sequence, including e.g., a promoter, ribosomal binding site, transcriptional or translational start and stop sequences, or of an enhancer or activator sequence.

A variety of methods of engineering a host cell can be employed to control or reduce expression of an endogenous polynucleotide, such as a gene encoding a Pat1 protein, including e.g., disrupting the polynucleotide encoding the Pat1 protein, disrupting the promoter which is operably linked to such polynucleotide, replacing such promoter with another promoter which has lower promoter activity, modifying or modulating (e.g., activating, up-regulating, inactivating, inhibiting, or down-regulating) regulatory sequences which modulate the expression of such polynucleotide, such as using respective transcription regulators targeted to the relevant sequences by an RNA guided ribonuclease used in a CRISPR based method of modifying a host cell, e.g., regulatory sequences selected from the group consisting of promoter, ribosomal binding sites, transcriptional start or stop sequences, translational start or stop sequences, enhancer or activator sequences, repressor or inhibitor sequences, signal or leader sequences, in particular those which control the expression of a protein.

Specifically, said one or more genetic modifications include one or more genomic mutations including deletion, replacement (i.e., substitution), or inactivation of a gene or genomic sequence, which reduces expression of a gene or part of a gene by at least 50%, 60%, 70%, 80%, 90%, or 95%, or even completely abolishes its expression, e.g., by a knockout of the gene, as compared to the respective host without such genetic modification.

According to a specific aspect, the control or reduction of said Pat1 protein expression is determined by the control or reduction of the amount (e.g., the level or concentration) of said Pat1 protein in the cell. Specifically, the amount of said Pat1 protein is determined by a suitable method, such as employing a Western Blot, immunofluorescence imaging, flow cytometry or mass spectrometry, in particular wherein mass spectrometry is liquid chromatography-mass spectrometry (LC-MS), or liquid chromatography tandem-mass spectrometry (LC-MS/MS) e.g., as described by Doneanu et al. (MAbs. 2012; 4(1): 24-44).

Specifically, the host cell is genetically modified to reduce the amount (e.g., the level or concentration) of said Pat1 protein, by at least any one of 20%, 30%, 40%, 50%, 60%, 70%, 80%, 90%, or 95%, (mol/mol) compared to the host cell without said modification, or even by 100% e.g., to a non-detectable amount, thereby completely abolishing production of the Pat1 protein, e.g., by a knockout of the respective coding gene.

According to a specific aspect, once the host cell described herein is cultured in a cell culture, the amount of total Pat1 protein in the host cell or host cell culture is reduced by at least any one of 20%, 30%, 40%, 50%, 60%, 70%, 80%, 90%, or 95%, (mol/mol), or even by 100% e.g., to a non-detectable amount, compared to a reference amount expressed or produced by the host cell prior to or without such genetic modification, or compared to a reference amount produced in a respective host cell culture, or compared to the host cell prior to or without said modification.

Specifically, PAT1 expression is controlled by said genetic modification to comprise a controlled PAT1 expression system, such that expression of the Pat1 protein in a host cell culture under carbon-source limiting or starving conditions is about the same (+/−20%) or less e.g., reduced expression (such as by at least any one of 20%, 30%, 40%, 50%, 60%, 70%, 80%, 90%, or 95% (mol/mol) reduced), as compared to the respective host cell prior to or without such genetic modification wherein the respective host cell is cultured under no carbon-source limiting or starving conditions. Thus, the host cell comprising the controlled pat1 expression system can be cultured under carbon-source limiting or starving conditions without significant losses in productivity due to a Pat1 promoted controlled translational activity, in particular a Pat1 mRNA controlled translational activity.

Specifically, the amount of P-bodies, in particular the number, abundance and/or size of P-bodies, is reduced by said genetic modification to control expression of the Pat1 protein as compared to the respective strains without such genetic modification.

Specifically, the amount of P-bodies is reduced by at least any one of 10%, 20%, 30%, 40%, 50%, 60%, 70%, 80%, 90%, or by 100%. The amount of P-bodies can be determined by fluorescent microscopy of cells expressing a P-body reporter construct (i.e., a P-body component such as Dcp2 fused to a fluorescent protein such as GFP or mCherry), and (automated) counting of the fluorescent foci of this reporter construct (as described in literature, e.g. by Sachdev et al. 2019. Elife 8:e41415. doi: 10.7554/eLife.41415.

Specifically, the host cell's translation activity is increased by said genetic modification to control expression of the Pat1 protein as compared to the respective strains without such genetic modification. Specifically, the host cell's translation activity is increased by at least any one of 1.15 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold.

Specifically, the host cell's translation activity is increased by 1.15-fold-12-fold, or 1.15-fold-10-fold, or 1.15-fold-8-fold, or 1.15-fold-6-fold, or 1.2-fold-12-fold, or 1.2-fold-10-fold, or 1.2-fold-8-fold, or 1.2-fold-6-fold, or 1.3-fold-12-fold, or 1.3-fold-10-fold, or 1.3-fold-8-fold, or 1.3-fold-6-fold.

When comparing the host cell described herein for the effect of said genetic modification to reduce production of said Pat1 protein, it is typically compared to the comparable host cell prior to or without such genetic modification. Comparison is typically made with the same host cell species or type without such genetic modification, which is engineered to produce the recombinant or heterologous POI, in particular when cultured under conditions to produce said POI. However, a comparison can also be made with the same host cell species or type which is not further engineered to produce the recombinant or heterologous POI.

According to a specific embodiment, the host cell is genetically modified to comprise one or more deletions of (one or more) genomic sequences, in particular genomic sequences encoding Pat1 protein. Such host cell is typically provided as a deletion or knockout strain.

According to a specific example, the endogenous gene encoding said Pat1 protein is knocked out. According to a specific example, the host cell comprises additional knockout of other decapping activators. Yet, according to a specific alternative example, the host cell does not comprise a knockout of dhh1.

According to a specific aspect, the one or more genetic modifications comprise genomic mutations which constitutively impair or reduce the expression of one or more endogenous polynucleotides.

According to another specific aspect, the one or more genetic modifications comprise genomic mutations which conditionally impair or reduce the expression of one or more endogenous polynucleotides e.g., by introducing one or more inducible or repressible regulatory sequences. Such conditionally active modifications are particularly targeting those regulatory elements and genes which are active and/or expressed dependent on cell culture conditions.

Specifically, the expression of a Pat1 protein is controlled or reduced in the host cell when producing the POI. Specifically, expression of said Pat1 protein is controlled or reduced under conditions of the host cell culture during which the POI is produced.

According to a specific aspect, the host cell comprises a recombinant expression cassette that expresses the GOI, which expression cassette is heterologous to the host cell or artificial, thus not naturally-occurring in the respective wild-type host cell.

Specifically, the host cell comprises only one or multiple expression cassettes such as at least 2, 3, 4, or 5 expression cassettes e.g., multiple copies of said expression cassettes, wherein a gene copy number (GCN) denotes the number of copies. For example, the host cell comprises up to 2, 3, 4, or five copies. Each of the copies may comprise or consist of the same or different sequences, and particularly includes a promoter operably linked to the GOI.

According to a specific aspect, the expression cassette comprises or consists of an artificial fusion of polynucleotides, including a promoter operably linked to the GOI, and optionally further sequences, such as a signal, leader, or a terminator sequence. Specifically, the expression cassette comprises or consists of an artificial fusion of a promoter, the GOI, and one or more additional regulatory sequences in operable linkage to allow expression of the GOI from said expression cassette for POI production.

Specifically, the expression cassette comprises one or more expression control sequences operably linked to said GOI, preferably said one or more expression control sequences comprise a promoter which is an inducible, de-repressible or otherwise regulatable promoter, or a constitutive promoter, preferably wherein the promoter is a carbon-source regulatable promoter such as any one of SEQ ID NO:38-51, or a promoter suitably used in a mammalian host cells (in particular a CHO host cell), such as a CMV (e.g., murine CMV, mCMV), a SV40 or a PGK promoter.

Specifically, within the expression cassette, the expression cassette promoter (ECP) and GOI are heterologous to each other, not occurring in such combination (or operable linkage) in nature e.g., wherein either one (or only one) of the promoter and GOI is artificial or heterologous to the other and/or to the host cell described herein; the promoter is an endogenous promoter and the GOI is a heterologous GOI; or the promoter is an artificial or heterologous promoter and the GOI is an endogenous GOI; wherein both, the promoter and GOI, are artificial, heterologous or from different origin, such as from a different species or type (strain) of cells compared to the host cell described herein. Specifically, the promoter of the expression cassette is not naturally associated with and/or not operably linked to said GOI in the cell which is used as a host cell described herein.

According to a specific aspect, ECP is inducible in the presence of a growth-limiting amount of a carbon source, in particular a carbon source which is not methanol (a “non-methanol carbon source”), preferably in the absence of methanol. Specifically, the GOI expression by the expression cassette is inducible by the inducible ECP.

Specifically, the ECP is repressible in the presence of an excess amount of a non-methanol carbon source which excess amount is higher than the growth-limiting amount.

Specifically, the expression cassette comprises a promoter which is inducible in the presence of a growth-limiting amount of up to 1 g/L of a carbon source; and repressible in the presence of an excess amount of a carbon source that is higher than the growth-limiting amount, preferably wherein the promoter comprises or consists of a regulatable promoter, such as any of the regulatable promoters described herein.

In particular, a regulatable promoter, such as a repressible and de-repressible (herein referred to as (de)repressible) carbon-source regulatable promoter, or inducible promoter can be used.

Specific examples of an inducible promoter include the native methanol-inducible pAOX1 or pAOX2 and functional variants thereof e.g., the native promoters pAOX1 (SEQ ID NO:22) or pAOX2 (SEQ ID NO:23), or any of the native methanol-inducible promoters of P. pastoris (e.g., SEQ ID NO:24-37, published by Gasser, Steiger, & Mattanovich, Microb Cell Fact. 2015, 14: 196).

Preferred regulatable promoters that are inducible by methanol are selected from promoter comprising at least any one of 60%, 65%, 70%, 75%, 80%, 85%, 90%, 95%, 96%, 97%, 98%, 99%, or 100% sequence identity to any one of, or to any one of SEQ ID NO:22-37.

Specific examples of a carbon-source regulatable promoter include pG1-pG8, and fragments thereof, as published in WO2013050551; any of the regulatable promoter, such as pG1 and pG1-x, as published in WO2017021541 A1. Specifically, any carbon source regulatable promoter may be used e.g., de-repressible promoters such as pG1-pG8 (pG1: SEQ ID NO:38, pG3: SEQ ID NO:41, pG4: SEQ ID NO:42, pG5: SEQ ID NO:43, pG7: SEQ ID NO:44, pG8: SEQ ID NO:45, and functional variants of any of the foregoing, such as fragments e.g., fragments of pG1, designated pG1a-pG1f: SEQ ID NO:46-51), orthe functional variants designated pG1-x, in particular pG1-3 (e.g., SEQ ID NO:39, such as referred to as pG1-D1240), or pG1-4 (e.g., SEQ ID NO:40, such as referred to as pG1-D1427), as published in WO2013050551 and WO2017021541, or a functional variant of any of the foregoing with a length of at least 300, 400, or 500 bp (in particular including the 3′-end).

Preferred regulatable promoters that are regulatable by a non-methanol carbon source are selected from promoter comprising at least any one of 60%, 65%, 70%, 75%, 80%, 85%, 90%, 95%, 96%, 97%, 98%, 99%, or 100% sequence identity to any one of SEQ ID NO:38-51, or SEQ ID NO:39-51.

Specifically, a functional variant of a promoter as described herein comprises at least any one of 60%, 65%, 70%, 75%, 80%, 85%, 90%, 95%, or 100% sequence identity to the promoter from which it is derived, over the full-length or the part at the 3′-end of the promoter sequence which part has a length of at least 300, 400, or 500 bp, and is functional to operatively control expression of the polynucleotide to be expressed, in particular with about the same promoter activity (e.g. +/−any one of 50%, 40%, 30%, 20%, or 10%), although the promoter activity may be improved as compared to the promoter from which it is derived. Specific functional promoter variants of pG1-3 or pG1-4 are those comprising at least two main regulatory regions and/or at least two core regulatory regions, and/or at least two T motifs, as indicated in FIG. 1 (SEQ ID NO:39 and SEQ ID NO:40, respectively).

Further examples of suitable promoter sequences are described in Prielhofer et al. (BMC Syst Biol. 2017. 11(1):123) and Mattanovich et al. (Methods Mol. Biol. (2012) 824:329-58) and include glycolytic enzymes like triosephosphate isomerase (TPI), phosphoglycerate kinase (PGK), glyceraldehyde-3-phosphate dehydrogenase (GAPDH or GAP) and variants thereof, lactase (LAC) and galactosidase (GAL), P. pastoris glucose-6-phosphate isomerase promoter (PPGI), the 3-phosphoglycerate kinase promoter (pPGK), the glycerol aldehyde phosphate dehydrogenase promoter (pGAP), translation elongation factor promoter (PTEF), and the promoters of P. pastoris enolase 1 (pEN01), triose phosphate isomerase (pTPI), ribosomal subunit proteins (pRPS2, pRPS7, pRPS31, pRPL1), alcohol oxidase promoter (pAOX1, pAOX2) or variants thereof with modified characteristics, the formaldehyde dehydrogenase promoter (pFLD), isocitrate lyase promoter (plCL), alpha-ketoisocaproate decarboxylase promoter (pTHI), the promoters of heat shock protein family members (pSSA1, pHSP90, pKAR2), 6-phosphogluconate dehydrogenase (pGND1), phosphoglycerate mutase (pGPM1), transketolase (pTKL1), phosphatidylinositol synthase (pPIS1), ferro-02-oxidoreductase (pFET3), high affinity iron permease (pFTR1), repressible alkaline phosphatase (pPH08), N-myristoyl transferase (pNMT1), pheromone response transcription factor (pMCM1), ubiquitin (pUB14), single-stranded DNA endonuclease (pRAD2), the promoter of the major ADP/ATP carrier of the mitochondrial inner membrane (pPET9) (WO2008/128701) and the formate dehydrogenase (FMD) promoter.

Further examples of suitable promoters include S. cerevisiae enolase (ENO1), S. cerevisiae galactokinase (GAL1), S. cerevisiae alcohol dehydrogenase and S. cerevisiae glyceraldehyde-3-phosphate dehydrogenase (ADH1, ADH2, GAP), S. cerevisiae triose phosphate isomerase (TPI), S. cerevisiae metallothionein (CUP1), and S. cerevisiae 3-phosphoglycerate kinase (PGK), and the maltase gene promoter (MAL).

Further examples of suitable ECPs are promoters suitably used in a mammalian expression system such as e.g., a CHO host cell. Exemplary promoters comprise or consist of a promoter sequence from any one of an SV40 promoter sequence, an CMV (e.g., mCMV) promoter sequence, or a PGK promoter sequence.

Specifically, the ECP has sufficient sequences, e.g., from a naturally occurring or engineered promoter such that operably linking a coding sequence to the promoter results in the expression of the coding sequence. For example, a cytomegalovirus (CMV) promoter comprises all or an active fragment of the CMV promoter, e.g., all or an active fragment of the CMV promoter including optionally intron A and/or UTR sequences. In an embodiment, a CMV promoter differs at no more than 5, 10, 20, 30, 50, or 100 nucleotides from a naturally occurring or engineered variant CMV promoter.

Specifically, the CMV promoter differs at no more than 1, 5, 10, or 50% of its nucleotides from a naturally occurring or engineered variant CMV promoter.

According to a specific example, the host cell is a CHO host cell, and the ECP comprises or consists of a CMV promoter.

According to another specific example, the host cell is a P. pastoris host cell, and the promoter is a carbon source regulatable promoter such as e.g., any one of SEQ ID NO:38-51.

Preferably, the ECP is carbon source regulatable, such as repressed in the presence of amounts higher than any one of 1, 1.5, 2, 2.5, or 3 g/L of a carbon source in the cell culture medium or supernatant (herein referred to as a promoter-repressing amount), and induced or de-repressed in the presence of no detectable carbon source or amounts up to any one of 0.1, 0.2, 0.3, 0.4, 0.5, 0.6, 0.7, 0.8, 0.9, or 1.0 g/L carbon source in the cell culture medium or supernatant (herein referred to as a promoter-inducing amount). Such amounts in the cell culture medium or supernatant are particularly understood as the amount which upon feeding of the host cell and consumption by the host cell may be detectable. Typically, when producing a POI under growth-limiting conditions, the cell culture is fed by adding a supplemental carbon source, yet in an amount that is immediately consumed by the cells during POI production, thus, leaving no or only a low remaining amount in the cell culture medium or supernatant e.g., an amount up to 1.0 g/L.

Specifically, the cell is fed in the production phase of a cell culture with the supplemental carbon source in an amount which is growth-limiting, wherein the cells are fed with the carbon source in the growth-limiting amount without inducing carbon source-starvation of the cells. In particular, carbon source-starvation is avoided in a method of POI production.

Specifically, the non-methanol carbon source is a carbohydrate.

Specifically, the non-methanol carbon source is selected from saccharides, polyols, alcohols, or mixtures of any one or more of the foregoing.

Specifically, the saccharides may be any one or more of monosaccharides, such as a hexose e.g., glucose, fructose, galactose or mannose, or a disaccharide, such as saccharose; or an alcohol or polyol e.g., ethanol, or any diol, or triol, e.g., glycerol, or a mixture of any of the foregoing. Specifically, any such non-methanol carbon source may be used in the cell culture in an amount to produce said POI under the control of the ECP.

According to a specific aspect, the ECP may be a constitutive promoter. Specific examples of constitutive promoter include e.g., the pGAP (e.g., SEQ ID NO:52, SEQ ID NO:53), any of the constitutive promoter such as pCS1 (e.g., SEQ ID NO:54, or functional variants thereof, such as published in WO2014139608), pMDH3 (e.g., SEQ ID NO:55), pPOR1 (e.g., SEQ ID NO:56), pRPP1B, pPDC1, pGPM1, pFBA1-1, or a functional variant of any of the foregoing.

Preferred constitutive promoters are selected from promoter comprising at least any one of 60%, 65%, 70%, 75%, 80%, 85%, 90%, 95%, 96%, 97%, 98%, 99%, or 100% sequence identity to any one of SEQ ID NO:52-56.

Specifically, the GOI expression cassette comprises the ECP operably linked to the GOI encoding the POI, and may further comprise signal and/or leader sequences, as necessary to express and produce the POI as a secreted protein.

According to a specific aspect, the GOI expression cassette further comprises a nucleotide sequence encoding a signal peptide enabling the secretion of the POI preferably wherein the nucleotide sequence encoding the signal peptide is fused adjacent to, or directly to the 5′-end of the GOI.

Specifically, the signal peptide is selected from the group consisting of signal sequences from S. cerevisiae alpha-mating factor prepro-peptide, the signal peptides from the P. pastoris acid phosphatase gene (PHO1) and the extracellular protein X (EPX1) (Heiss, S., V. Puxbaum, C. Gruber, F. Altmann, D. Mattanovich & B. Gasser, Microbiology 2015; 161(7):1356-68).

Specifically, any of the signal and/or leader sequences as described in WO2014067926 A1 can be used, in particular SEQ ID NO:57 or SEQ ID NO:58.

Specifically, signal sequences as described in WO2012152823 A1 can be used, in particular the signal sequence of native alpha mating factor of S. cerevisiae identified as SEQ ID NO:59, or functional variants thereof.

According to a specific aspect, the GOI expression cassette is comprised in an autonomously replicating vector or plasmid, or integrated within a chromosome of said host cell.

The expression cassette may be introduced into the host cell and integrated into the host cell genome (or any of its chromosomes) as intrachromosomal element e.g., at a specific site of integration or randomly integrated, whereupon a high producer host cell line is selected. Alternatively, the expression cassette may be integrated within an extrachromosomal genetic element, such as a plasmid or an artificial chromosome e.g., a yeast artificial chromosome (YAC). According to a specific example, the expression cassette is introduced into the host cell by a vector, in particular an expression vector, which is introduced into the host cell by a suitable transformation technique. For this purpose, the GOI may be ligated into an expression vector.

A preferred yeast expression vector (which is preferably used for expression in yeast) is selected from the group consisting of plasmids derived from pPICZ, pGAPZ, pPIC9, pPICZalfa, pGAPZalfa, pPIC9K, pGAPHis, pPUZZLE or GoldenPiCS.

Techniques for transfecting or transforming host cells for introducing a vector or plasmid are well known in the art. Reference is for example made to the standard handbooks, such as Sambrook et al., 2012, Molecular Cloning: A Laboratory Manual, volumes 1-4, Cold Spring Harbor Press, NY), and in other molecular biology manuals.

These can include electroporation, spheroplasting, lipid vesicle mediated uptake, heat shock mediated uptake, calcium phosphate mediated transfection (calcium phosphate/DNA co-precipitation), viral infection, and particularly using modified viruses such as, for example, modified adenoviruses, microinjection and electroporation.

As used herein, the term “transforming” a eukaryotic cell is understood to encompass “transfecting” the same.

Transformants as described herein can be obtained by introducing the expression cassette, vector or plasmid DNA into a host and selecting transformants which express the relevant protein or selection marker. Host cells can be treated to introduce heterologous or foreign DNA by methods conventionally used for transformation of host cells, such as the electric pulse method, the protoplast method, the lithium acetate method, and modified methods thereof. P. pastoris is preferably transformed by electroporation. Preferred methods of transformation for the uptake of the recombinant DNA fragment by the microorganism include chemical transformation, electroporation or transformation by protoplastation.

According to a specific aspect, the host cell described herein may undergo one or more further genetic modifications e.g., for improving protein production.

Specifically, the host cell is further engineered to modify one or more genes influencing proteolytic activity used to generate protease deficient strains, in particular a strain deficient in carboxypeptidase Y activity. Particular examples are described in WO1992017595A1. Further examples of a protease deficient Pichia strain with a functional deficiency in a vacuolar protease, such as proteinase A or proteinase B, are described in U.S. Pat. No. 6,153,424A. Further examples are Pichia strains which have an ade2 deletion, and/or deletions of one or both of the protease genes, PEP4 and PRB1, are provided by e.g., ThermoFisher Scientific.

Specifically, the host cell is engineered to modify at least one nucleic acid sequence encoding a functional gene product, in particular a protease, selected from the group consisting of PEP4, PRB1, YPS1, YPS2, YMP1, YMP2, YMP1, DAP2, GRHI, PRD1, YSP3, and PRB3, as disclosed in WO2010099195A1.

Overexpression or underexpression of genes encoding helper factors is specifically applied to enhance expression of a GOI e.g., as described in WO2015158800A1 or WO2022069613A1.

Overexpression of the following genes was shown to increase POI secretion in P. pastoris: PP7435_Chr3-0607, PP7435_Chr3-0933, PP7435_Chr2-0220, PP7435_Chr3-0639, PP7435_Chr4-0108, PP7435_Chr1-1232, PP7435_Chr1-1225, PP7435_Chr1-0667, and PP7435_Chr4-0448.

Underexpression of the following genes was shown to increase POI secretion in P. pastoris: PP7435_Chr1-0176, PP7435_Chr3-1062, and PP7435_Chr4-0252.

The POI can be any one of eukaryotic, prokaryotic or synthetic peptides, polypeptides, proteins (including e.g., fusion proteins), or metabolites of a host cell.

Specifically, the POI is heterologous to the host cell species.

Specifically, the POI is a secreted peptide, polypeptide, or protein i.e., secreted from the host cell into the cell culture supernatant.

Specifically, the POI is a heterologous peptide, polypeptide, protein or fusion protein.

Specifically, the POI is a secreted POI i.e., secreted from the host cell.

Specifically, the POI is a eukaryotic protein, preferably a mammalian derived or related protein such as a human protein or a protein comprising a human protein sequence, or a bacterial protein or bacterial derived protein

Preferably, the POI is a therapeutic or diagnostic protein or product, such as functioning in mammals e.g., a human therapeutic or diagnostic.

In specific cases, the POI is a multimeric protein, specifically a dimer or tetramer.

According to a specific aspect, the POI is selected from the group consisting of an antigen-binding protein, an enzyme, a peptide, a protein antibiotic, a toxin fusion protein, a structural protein, a regulatory protein, a protein vaccine antigen, a hormone, a growth factor, a cytokine, and a blood clotting or coagulation factor.

According to a specific aspect, the POI is a peptide, polypeptide or protein selected from the group consisting of an antigen-binding protein, an enzyme, a peptide, a protein antibiotic, a toxin fusion protein, a carbohydrate—protein conjugate, a structural protein, a regulatory protein, a vaccine antigen, a growth factor, a hormone, a cytokine, a process enzyme, and a metabolic enzyme.

Specifically, the POI is a process enzyme or metabolic enzyme.

Specifically, the antigen-binding protein is an antibody molecule.

Specifically, the antibody molecule is a monoclonal antibody.

Specifically, the antibody molecule is a full-length antibody, an antibody comprising one or more epitope binding fragments of a full-length antibody, or a bispecific or multi-specific antibody comprising one or more of said fragments.

Specifically, said one or more epitope binding fragments of a full-length antibody are Fab, Fab′, F(ab′)₂, Fv, or scFv fragments, or single domain antibodies.

Specifically, the antigen-binding protein is selected from the group consisting of:
a) antibodies or antibody fragments, such as any of chimeric antibodies, humanized antibodies, bi-specific antibodies, Fab, Fd, scFv, diabodies, triabodies, Fv tetramers, minibodies, single-domain antibodies like VH, VHH, IgNARs, or V-NAR, in particular camelid VHH:
b) antibody mimetics, such as Adnectins, Affibodies, Affilins, Affimers, Affitins, Alphabodies, Anticalins, Avimers, DARPins, Fynomers, Kunitz domain peptides, Monobodies, or NanoCLAMPS; or
c) fusion proteins comprising one or more immunoglobulin-fold domains, antibody domains or antibody mimetics.

A specific POI is an antigen-binding molecule such as an antibody, or a fragment thereof, in particular an antibody fragment comprising an antigen-binding domain. Among specific POls are antibodies such as monoclonal antibodies (mAbs), immunoglobulin (Ig) or immunoglobulin class G (IgG), heavy-chain antibodies (HcAb's), or fragments thereof such as fragment-antigen binding (Fab), Fd, single-chain variable fragment (scFv), or engineered variants thereof such as for example Fv dimers (diabodies), Fv trimers (triabodies), Fv tetramers, or minibodies and single-domain antibodies like VH, VHH, IgNARs, or V-NAR, or any protein comprising an immunoglobulin-fold domain. Further antigen-binding molecules may be selected from antibody mimetics, or (alternative) scaffold proteins such as e.g., engineered Kunitz domains, Adnectins, Affibodies, Affiline, Anticalins, or DARPins.

Specifically, the POI is heterologous to the host cell species.

Specifically, the POI is a secreted peptide, polypeptide, or protein i.e., secreted from the host cell into the cell culture supernatant.

Specifically, the GOI is expressed with a secretion signal sequence, preferably wherein the secretion signal peptide (or a leader comprising a secretion signal peptide) is fused to the N-terminus of the POI.

According to a specific aspect, the host cell can be any eukaryotic cell such as an animal cell, a vertebrate cell, a mammalian cell such as a mammalian non-human animal cell (e.g., a CHO cell) or a human cell, a plant cell, a nematodal cell, an invertebrate cell such as an insect cell or a mollusc cell, a stem cell derived of any of the foregoing, or a fungal cell, or a yeast cell.

Specifically, the host cell is a cell of a genus selected from the group consisting of Pichia, Hansenula, Komagataella, Saccharomyces, Kluyveromyces, Candida, Ogataea, Yarrowia, and Geotrichum, specifically Saccharomyces cerevisiae, Pichia pastoris, Ogataea minuta, Kluyveromces lactis, Kluyveromes marxianus, Yarrowia lipolytica or Hansenula polymorpha, or of filamentous fungi like Aspergillus awamori or Trichoderma reesei. Preferably, the host cell is a methylotrophic yeast, preferably Pichia pastoris. Herein Pichia pastoris is used synonymously for any one or more or all of Komagataella pastoris, Komagataella phaffii and Komagataella pseudopastoris.

According to a specific aspect, the host cell is
a) a yeast cell of a genus selected from the group consisting of Pichia, Hansenula, Komagataella, Saccharomyces, Kluyveromyces, Candida, Ogataea, Yarrowia, and Geotrichum, such as of a Pichia genus (e.g. Pichia pastoris, Pichia methanolica, Pichia kluyveri, and Pichia angusta), Komagataella genus (e.g., Komagataella pastoris, Komagataella pseudopastoris or Komagataella phaffii), Saccharomyces genus (e.g. Saccharomyces cerevisae, Saccharomyces kluyveri, Saccharomyces uvarum), Kluyveromyces genus (e.g. Kluyveromyces lactis, Kluyveromyces marxianus), the Candida genus (e.g. Candida utilis, Candida cacaoi, Candida boidinii,), the Geotrichum genus (e.g. Geotrichum fermentans), Hansenula polymorpha, Yarrowia lipolytica, or Schizosaccharomyces pombe; or
b) a cell of filamentous fungi, such as Aspergillus awamori or Trichoderma reesei.

Preferred is the species Pichia pastoris. Specifically, the host cell is a Pichia pastoris strain selected from the group consisting of CBS 704, CBS 2612, CBS 7435, CBS 9173-9189, DSMZ 70877, X-33, GS115, KM71, KM71H and SMD1168.

Sources: CBS 704 (=NRRL Y-1603=DSMZ 70382), CBS 2612 (=NRRL Y-7556), CBS 7435 (=NRRL Y-11430), CBS 9173-9189 (CBS strains: CBS-KNAW Fungal Biodiversity Centre, Centraalbureau voor Schimmelculturen, Utrecht, The Netherlands), and DSMZ 70877 (German Collection of Microorganisms and Cell Cultures); strains from Invitrogen, such as X-33, GS115, KM71, KM71 H and SMD1168.

Examples of preferred S. cerevisiae strains include W303, CEN.PK and the BY-series (EUROSCARF collection). All of the strains described above have been successfully used to produce transformants and express heterologous genes.

According to a specific aspect, the eukaryotic host cell can be a fungal cell (e.g., Aspergillus (such as A. niger, A. fumigatus, A. oryzae, A. nidulans), Acremonium (such as A. thermophilum), Chaetomium (such as C. thermophilum), Chrysosporium (such as C. thermophile), Cordyceps (such as C. militaris), Corynascus, Ctenomyces, Fusarium (such as F. oxysporum), Glomerella (such as G. graminicola), Hypocrea (such as H. jecorina), Magnaporthe (such as M. oryzae), Myceliophthora (such as M. thermophile), Nectria (such as N. haematococca), Neurospora (such as N. crassa), Penicillium, Sporotrichum (such as S. thermophile), Thielavia (such as T. terrestris, T. heterothallica), Trichoderma (such as T. reesei), or Verticillium (such as V. dahlia)).

According to a specific aspect, the mammalian cell is a human or rodent or bovine cell, cell line or cell strain. Examples of specific mammalian cells suitable as host cells described herein are mouse myeloma (NSO)-cell lines, Chinese hamster ovary (CHO)-cell lines, HT1080, H9, HepG2, MCF7, MDBK Jurkat, MDCK, NIH3T3, PC12, BHK (baby hamster kidney cell), VERO, SP2/0, YB2/0, YO, C127, L cell, COS, e.g., COS1 and COS7, QC1-3, HEK-293, VERO, PER.C6, HeLA, EBI, EB2, EB3, oncolytic or hybridoma-cell lines. Preferably the mammalian cells are CHO-cells or respective cell lines. In one embodiment, the cell is a CHO cell. Examples of CHO cells include, but are not limited to, CHOK1, CHOK1SV, Potelligent CHOK1SV, CHO GS knockout, CHOK1SV GS-KO, CHOS, CHO DG44, CHO DXB11, CHOZN, or a CHO-derived cell.

In one embodiment, the cell is a CHO-K1 cell, a CHO-K1 SV cell, a DG44 CHO cell, a DUXB11 CHO cell, a DUKX CHO cell, a CHO-S, a CHO FUT8 knock-out CHO GS knock-out cell, a CHO FUT8 GS knock-out cell, a CHOZN, or a CHO-derived cell. The CHO GS knock-out cell (e.g., GSKO cell) is, for example, a CHO-K1 SV GS knockout cell. The CHO FUT8 knockout cell is, for example, the Potelligent® CHOK1 SV (Lonza Biologics, Inc.). Eukaryotic cells also include avian cells, cell lines or cell strains, such as for example, EBx® cells, EB14, EB24, EB26, EB66, or EBvl3.

According to another specific aspect, the eukaryotic cell is an insect cell (e.g., Sf9, Mimic™ Sf9, Sf21, High Five™ (BT1-TN-5B1-4), or BT1-Ea88 cells), an algae cell (e.g., of the genus Amphora, Bacillariophyceae, Dunaliella, Chlorella, Chlamydomonas, Cyanophyta (cyanobacteria), Nannochloropsis, Spirulina, or Ochromonas), or a plant cell (e.g., cells from monocotyledonous plants (e.g., maize, rice, wheat, or Setaria), or from a dicotyledonous plants (e.g., cassava, potato, soybean, tomato, tobacco, alfalfa, Physcomitrella patens or Arabidopsis).

Suitable host cells are commercially available, for example, from culture collections such as the DSMZ (Deutsche Sammlung von Mikroorganismen and Zellkulturen GmbH, Braunschweig, Germany) or the American Type Culture Collection (ATCC).

According to a specific aspect, the host cell is a production host cell and particularly provided as a production host cell line. Specifically, the production host cell is a differentiated form of cells. The production host cell can be derived from a primary cell in culture.

The invention further provides for a method for producing a host cell as described herein, comprising genetic engineering of a host cell to (i) introduce said recombinant expression cassette expressing said GOI, and to (ii) control expression of a Pat1 protein. such as to control, in particular to reduce or increase expression of the Pat1 protein, e.g., as compared to a cell of the same type in which the level of Pat1 protein has not been controlled, or compared to an endogenous expression thereof.

The invention further provides for a method of increasing the yield and/or titer of producing a heterologous protein of interest (POI) in a host cell or in a host cell culture, under carbon source limiting conditions, by engineering the host cell to control expression of a Pat1 protein in the host cell, such as to control, in particular to reduce or increase expression of the Pat1 protein, e.g., as compared to a cell of the same type in which the level of Pat1 protein has not been controlled, or compared to an endogenous expression thereof.

Specifically, the methods described herein are characterized by the genetic engineering of the host cell and one or more of the respective features of the host cell, as further described herein with respect to the host cell of the invention.

Specifically, the methods described herein produce or employ a recombinant host cell as further described herein. Specifically, the host cell is characterized and/or engineered as further described herein.

According to a specific embodiment, the methods described herein refer to a host cell which is a P. pastoris host cell, wherein the expression of the Pat1 protein is reduced by inactivating expression of the Pat1 protein in the host cell, in particular wherein the POI is a heterologous, secreted POI.

Specifically provided herein is a method of increasing the yield of a secreted heterologous POI in a P. pastoris host cell under carbon source limiting conditions, comprising inactivating expression of the Pat1 protein in the host cell.

Specifically, the method for producing a host cell as described herein, is characterized by one or more of the following features:
a) an at least 1.2-fold increased product yield and/or titer and/or an at least 1.1-fold or at least 1.2-fold increased specific productivity (μg/g cell dry mass (CDM)) per hour, and/or an at least 1.2-fold increased volumetric productivity (μg/L per hour) for said POI, compared to the host cell without said genetic modification to control expression of the Pat1 protein;
b) the product yield and/or titer, the specific productivity or volumetric productivity for said POI is increased in a host cell culture under carbon-source limiting conditions; preferably wherein the carbon-source limiting conditions are characterized by the presence of a growth-limiting amount of a carbon source in the host cell culture, which is up to 1 g/L;
c) the genetic engineering comprises knocking out at least one or all copies of a gene encoding the Pat1 protein from the genome of the cell or a regulatory nucleic acid sequence operably linked to the gene encoding the Pat1 protein, preferably by a split-marker cassette method, or by a method using a CRISPR-Cas9 protein in combination with an RNA specific for the gene encoding the Pat1 protein or specific to the regulatory nucleic acid sequence.

Specifically, the method of increasing the yield and/or titer of producing a heterologous POI in a host cell under carbon source limiting conditions as described herein, is characterized by one or more of the following features:
a) the carbon-source limiting conditions are characterized by the presence of a growth-limiting amount of a carbon source in the host cell culture, which is up to 1 g/L;
b) the yield and/or titer is at least 1.1-fold or at least 1.2-fold increased compared to the host cell without said genetic modification to control expression of the Pat1 protein;
c) the specific productivity (μg/g cell dry mass (CDM)) per hour and/or volumetric productivity (μg/L per hour) for said POI is at least 1.1-fold or at least 1.2-fold increased, compared to the host cell without said genetic modification to control expression of the Pat1 protein;
d) the genetic engineering comprises knocking out at least one or all copies of a gene encoding the Pat1 protein from the genome of the cell or a regulatory nucleic acid sequence operably linked to the gene encoding the Pat1 protein, by a split-marker cassette method, or by a method using a CRISPR-Cas9 protein in combination with an RNA specific for the gene encoding the Pat1 protein or specific to the regulatory nucleic acid sequence.

Specifically, any of the methods is characterized by the host cell's specific productivity (μg/g cell dry mass (CDM), in particular yeast dry mass (YDM) for yeast cells) per hour and/or volumetric productivity (μg/L per hour) for said POI, which is at least 1.1-fold or at least 1.2-fold increased, compared to the host cell without said genetic modification to control expression of the Pat1 protein.

Productivity and its fold change can be determined e.g., on a small scale or large scale, such as described in the Examples disclosed herein. For example, an increase in recombinant protein production might be determined at small-scale by measuring the concentration in the culture medium by a respective immunoassay, such as an ELISA. It can also be determined quantitatively by the ForteBio Octet method, or by HPLC.

Specific methods for determining the amount of POI production described herein, can refer to the specific production rate (qP) of the POI in the cell or cell culture, and/or to a time integral of a viable cell concentration (IVC). Specifically, the method may include the combination of determining qP and IVC. Recombinant POI production or productivity, being defined as concentration of the polypeptide in the culture medium, is typically understood as a function of these two parameters (qP and IVC).

Specifically, the host cell's specific productivity (μg/g cell dry mass (CDM), in particular yeast dry mass (YDM) for yeast cells) per hour and/or volumetric productivity (μg/L per hour) is increased by at least any one of 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold, as compared to the comparable host cell expressing said GOI, without such engineering for controlled or reduced Pat1 expression.

Specifically, the host cell's specific productivity (μg/g cell dry mass (CDM), in particular yeast dry mass (YDM) for yeast cells) per hour and/or volumetric productivity (μg/L per hour) is increased by 1.1-fold-12-fold, or 1.1-fold-10-fold, or 1.1-fold-8-fold, or 1.1-fold-6-fold, or 1.2-fold-12-fold, or 1.2-fold-10-fold, or 1.2-fold-8-fold, or 1.2-fold-6-fold, or 1.3-fold-12-fold, or 1.3-fold-10-fold, or 1.3-fold-8-fold, or 1.3-fold-6-fold.Specifically, the POI yield and/or titer is increased by at least any one of 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3 fold, 3.5 fold, 4 fold, 5 fold, 5.5 fold, 6 fold, 6.5 fold, 7 fold, 7.5 fold, 8 fold, 8.5 fold, 9 fold, 9.5 fold, 10 fold, 10.5 fold, 11 fold, 11.5 fold, or 12 fold, as compared to the comparable host cell expressing said GOI, without such engineering for controlled or reduced Pat1 expression.

Specifically, the POI yield or titer is increased by 1.1-fold-12-fold, or 1.1-fold-10-fold, or 1.1-fold-8-fold, or 1.1-fold-6-fold, or 1.2-fold-12-fold, or 1.2-fold-10-fold, or 1.2-fold-8-fold, or 1.2-fold-6-fold, or 1.3-fold-12-fold, or 1.3-fold-10-fold, or 1.3-fold-8-fold, or 1.3-fold-6-fold.

Specifically, the host cell's specific productivity (μg/g cell dry mass (CDM), in particular yeast dry mass (YDM) for yeast cells) per hour and/or volumetric productivity (μg/L per hour) and/or POI yield and/or titer is increased when producing the POI in a host cell culture under carbon source-limiting conditions. Specifically, the host cell is cultured under carbon source-limiting conditions to produce said POI, preferably in the presence of a growth-limiting amount of a carbon source which is up to 1 g/L.

According to a specific aspect, the invention provides for a method for producing a protein of interest (POI) encoded by a gene of interest (GOI) by culturing the recombinant host cell as further described herein under conditions to produce said POI.

Specifically, the method for producing the POI described herein is characterized by the genetic engineering of the host cell and the respective features of the host cell, as further described herein with respect to the host cell of the invention.

Specifically, the method for producing the POI described herein is characterized by one or more of the following features:
a) the host cell is cultured in a host cell culture under carbon-source limiting conditions to produce said POI, preferably wherein the carbon-source limiting conditions are characterized by the presence of a growth-limiting amount of a carbon source in the host cell culture, which is up to 1 g/L;
b) the endogenous expression of a Pat1 protein is reduced in the host cell, to a level or amount that increases the yield and/or titer of said POI, and/or the host cell's specific productivity for said POI (μg/g cell dry mass (CDM)) per hour and/or volumetric productivity for said POI (μg/L per hour);
c) the product yield and/or titer, the specific productivity or volumetric productivity for said POI is at least 1.2-fold increased compared to the host cell without said genetic modification to control expression of the Pat1 protein.

According to a specific aspect, the invention provides for the use of the host cell described herein for the production of a POI.

According to a further specific aspect, the invention provides for a method for producing a protein of interest (POI) in a host cell, comprising the steps:
a) genetically engineering the host cell to control or reduce endogenous expression of a Pat1 protein in the host cell;
b) introducing into the host cell a GOI expression cassette;
c) culturing said host cell under conditions to produce said PO;
d) optionally isolating said POI from the cell culture; and
e) optionally purifying said POI.

Specifically, step a) of the method described herein is carried out before, or after, or concomitantly with step b).

According to a specific aspect, the host cell is first genetically modified to control or reduce expression of said Pat1 protein before being engineered for producing the POI. According to a specific example, a wild-type host cell is genetically modified according to step a) of the method described herein. Specifically, the host cell is provided upon introducing said one or more genetic modifications into a wild-type host cell strain for control or reduction of said Pat1 protein.

According to a further aspect, the host cell is first engineered for producing the heterologous or recombinant POI, before being further genetically modified to control or reduce said Pat1 protein. According to a specific example, a wild-type host cell may first be engineered to comprise the expression cassette for POI production. Such engineered host cell may then be further modified to control or reduce said Pat1 protein, as described herein.

According to a further aspect, the host cell is undergoing both, the engineering for POI production and genetically modifying for control or reduction of said Pat1 protein in one method step e.g., employing the respective expression cassette, reagents and tools in one or more reaction mixtures.

Specifically, the method employs method steps to produce the recombinant host cell as further described herein.

Specifically, the host cell is cultured in a culture medium under conditions to secrete said POI into the host cell culture, and the POI is recovered from the host cell culture.

Specifically, the POI can be produced by culturing the host cell in an appropriate medium, isolating the expressed POI from the cell culture, in particular from the cell culture supernatant or medium upon separating the cells, and purifying it by a method appropriate for the expressed product, in particular upon separating the POI from the cell and purifying by suitable means. Thereby, a purified POI preparation can be produced.

Specifically, in said host cell culture, expression of a Pat1 protein is reduced to a level that increases the host cell's specific productivity for said POI (μg/g cell dry mass (CDM), in particular yeast dry mass (YDM) for yeast cells) per hour and/or volumetric productivity for said POI (μg/L per hour).

Specifically, the host cell is a cell line cultured in a cell culture, in particular a production host cell line.

According to a specific embodiment, the cell line is cultured under suitable batch, fed-batch or continuous culture conditions. The culture may be performed in microtiter plates, shake-flasks, or a bioreactor, and optionally starting with a batch phase as the first step, followed by a fed-batch phase or a continuous culture phase as the second step.

According to a specific aspect, the method described herein comprises a growing phase and a production phase.

Specifically, the method comprises the steps:
a) culturing the host cell under growing conditions (growing phase, or “growth phase”); and a further step
b) culturing the host cell under growth-limiting conditions (production phase), during which the GOI is expressed to produce said POI.

Specifically, the second step b) follows the first step a).

Specifically, the host cell is cultured in the first step under growing conditions in a cell culture medium comprising a basal carbon source e.g., in an amount sufficient to enable growth of the host cell in cell culture, optionally until the amount of the carbon source is consumed, and further culturing can be under growth-limiting conditions e.g., using a supplemental carbon source.

Specifically, said basal and/or supplemental carbon source is/are selected from saccharides, polyols, alcohols, or mixtures of any one or more of the foregoing.

According to a specific embodiment, the basal carbon source is different from the supplemental carbon source e.g., quantitatively and/or qualitatively different. The quantitative difference typically provides for the growth-limiting amount of the carbon source in a feed medium as used during the POI production phase.

According to a further specific embodiment, the basal and the supplemental carbon sources comprise the same type of molecules or carbohydrates, preferably in different concentrations. According to a further specific embodiment, the carbon source is a mixture of two or more different carbon sources.

Any type of organic carbon source may be used, in particular those typically used for host cell culture, in particular for eukaryotic host cell culture. According to a specific embodiment, the carbon source is a hexose, such as glucose, fructose, galactose or mannose, a disaccharide, such as saccharose, an alcohol, such as glycerol or ethanol, or a mixture thereof.

According to a specifically preferred embodiment, the basal carbon source is selected from the group consisting of glucose, glycerol, ethanol, or mixtures thereof. According to a preferred embodiment, the basal carbon source is glycerol.

According to a further specific embodiment, the supplemental carbon source is a hexose such as glucose, fructose, galactose and mannose, a disaccharide, such as saccharose, an alcohol, such as glycerol or ethanol, or a mixture thereof. According to a preferred embodiment, the supplemental carbon source is glucose.

Specifically,
a) the basal carbon source is selected from the group consisting of glucose, glycerol, ethanol, a mixture thereof; and
b) the supplemental carbon source is a hexose such as glucose, fructose, galactose or mannose, a disaccharide, such as saccharose, an alcohol, such as glycerol or ethanol, or a mixture of any of the foregoing.

Specifically, the feed medium is chemically defined and methanol-free.

Specifically, the second step b) employs a feed medium that provides for the supplemental carbon source in a growth limiting amount to keep the specific growth rate up to or below any one of 0.2 h⁻¹, 0.15 h⁻¹, 0.1 h⁻¹, 0.05 h⁻¹, 0.01 h⁻¹, 0.005 h⁻¹, 0.001 h-1, 0.0005 h⁻¹, or 0.0001 h⁻¹, specifically at least any one of 0.0001 h⁻¹, 0.0005 h⁻¹, 0.001 h, or 0.005 h⁻¹, specifically within the range of 0.0001 h⁻¹ to 0.2 h⁻¹, preferably 0.005 h⁻¹ to 0.2 h⁻¹, or 0.01 h⁻¹ to 0.2 h⁻¹, or 0.05 h⁻¹ to 0.2 h⁻¹, or 0.1 h⁻¹ to 0.2 h⁻¹, 0.15 h⁻¹ to 0.2 h⁻¹, or 0.005 h⁻¹ to 0.15 h⁻¹, or 0.01 h⁻¹ to 0.15 h⁻¹, or 0.05 h⁻¹ to 0.15 h⁻¹, or 0.1 h⁻¹ to 0.15 h⁻¹.

The feed medium may be added to the culture medium in the liquid form or else in an alternative form, such as a solid e.g., as a tablet or other sustained release means, or a gas. Yet, according to a preferred embodiment the limited amount of a supplemental carbon source added to the cell culture medium, may even be zero. Preferably, under conditions of a limited carbon substrate, the detectable concentration of a supplemental carbon source in the culture medium is 0-1 g/L, preferably less than any one of 0.9, 0.8, 0.7, 0.6, 0.5, 0.4, 0.3, 0.2, or 0.1 g/L, preferably less than any one of 90, 80, 70, 60, 50, 40, 30, 20, or 10 mg/L, or even less than 9, 8, 7, 6, 5, 4, 3, 2, or 1 mg/L, or specifically 1-50 mg/L, or 1-10 mg/L, specifically preferred 1 mg/L or even below, such as below the detection limit as measured with a suitable standard assay, e.g. determined as a residual concentration in the culture medium upon consumption by the growing cell culture.

In a preferred method, the limited amount of the supplemental source provides for a residual amount in the cell culture which is below the detection limit as determined in the fermentation broth at the end of a production phase or in the output of a fermentation process, preferably upon harvesting the fermentation product.

Specifically, said step a) culturing is performed in a batch phase; and said step b) culturing is performed in fed-batch or a continuous cultivation phase.

Specifically, the host cells are grown in a carbon source rich medium comprising a basal carbon source during the phase of high growth rate (under growing conditions), step a) (e.g. at least 50%, or at least 60%, at least 70%, at least 80%, at least 90%, at least 95%, at least 98%, at least 99%, or up to the maximum growth rate) and producing the POI during a phase of low growth rate (under growth-limiting conditions), step b) (e.g. less than 90%, preferably less than 80%, less than 70%, less than 60%, less than 50%, or less than 40%, less than 30%, less than 20%, less than 10%, less than 5%, less than 3%, less than 2%, less than 1%, less than 0.5%, less than 0.4%, less than 0.3%, or less than 0.2% of the maximum growth rate) while limiting the carbon source, in particular by feeding a defined minimal medium comprising only the amount of carbon source which is completely consumed when maintaining the cell culture in the production phase.

Specifically, the POI is expressed under said growth-limiting conditions e.g., by cultivating the cell line at a growth rate of less than the maximal growth rate, typically less than 90%, preferably less than 80%, less than 70%, less than 60%, less than 50%, less than 40%, less than 30%, less than 20%, less than 10%, less than 5%, less than 3%, less than 2%, less than 1%, less than 0.5%, less than 0.4%, less than 0.3%, or less than 0.2% of the maximum growth rate of the cells. Typically, the maximum growth rate is individually determined for each type of host cell.

Specifically, the batch phase is performed until a basal carbon source that is initially added to the cell culture is consumed by the cell line. The dissolved oxygen (DO) spike method can be used to determine basal carbon source consumption during batch phase.

According to a specific embodiment, the batch phase is characterized by a continuous decrease in oxygen partial pressure (pO₂) signal and wherein the end of the batch phase is characterized by an increase of pO₂. Typically, while consuming the basal carbon source during the batch phase and without adding further carbon sources as typical for batch phases, the oxygen partial pressure (pO₂) signal will continuously decrease until for example below 65% such as for example 30%.Upon consumption of the basal carbon source, the pO₂ may increase to e.g. above 30% such as for example above 65%, or more indicating the appropriate time point to switch to the fed-batch system using feed medium to add further carbon source under carbon source limited conditions.

Specifically, the pO₂ is decreased to less than 65% or less saturation during batch phase followed by an increase of above 65% or more saturation at the end of the batch. Specifically, the batch phase is performed until an increase of the oxygen partial pressure (pO₂) signal above 65% saturation, specifically above any of 70%, 75%, 80%, or 85%.

Specifically, the batch phase is performed for around 10 to 36h.

The term “around” with respect to cultivation time shall mean+/−5% or +/−10%.

For example, the specific batch performance time of around 10 to 36 h may be 18 to 39.6h, specifically 19 to 37.8h.

According to a specific embodiment, the batch phase is performed using 40 to 50 g/L glycerol, specifically 45 g/L glycerol as a basal carbon source in batch media, and cultivation is performed at 25° C. for around 27 to 30h, or at 30° C. for around 23 to 36h, or at any temperature between 25° C. and 30° C. during a cultivation time of 23 to 36h. Lowering the glycerol concentration in the batch medium would decrease the length of the batch phase, while increasing the glycerol in the batch medium would even prolong the batch phase. As an alternative to glycerol, glucose can be used e.g., in about the same amounts.

In a typical system of cell culture and POI expression, wherein a batch phase is followed by a fed-batch phase, specifically, the cultivation in the fed-batch phase is performed for any one of around 15 to 80h, around 15 to 70h, around 15 to 60h, around 15 to 50h, around 15 to 45h, around 15 to 40h, around 15 to 35h, around 15 to 30h, around 15 to 35h, around 15 to 25h, or around 15 to 20h; preferably around 20 to 40h. Specifically, the cultivation in the fed-batch phase is performed for any one of around 80h, around 70h, around 60h, around 55h, around 50h, around 45h, around 40h, around 35h, around 33h, around 30h, around 25h, around 20h, or around 15h.

Any fed-batch cultivation of less than 120 h or less than 100 h or up to 80h, which results in a successful POI production thereby obtaining a high yield and/or titer is herein referred to as “speed fermentation”. Specifically, the volume specific product formation rate (rP) is the amount of product (mg) formed per Unit Volume (L) and Unit time (h) (mg (L h)⁻¹). Volume specific product formation rate is also called space time yield (STY) or volumetric productivity.

Specifically, the fed-batch cultivation of the method described herein is performed such that a space time yield of around 30 mg (L h)⁻¹ (meaning 30 mg (L h)⁻¹+/−5% or +/−10%). Specifically, a space time yield of around 30 mg (L h)⁻¹ is achieved within around 30 h fed batch, specifically at least any of 27, 28, 29, 30, 31, 32, or 33 mg (L h)⁻¹ within less than any one of 33h, 32h, 31h, 30h, 29h, 28h, 27h, 26h, or 25 h fed batch time can be achieved.

Specifically, the batch phase is performed as a first step a), and the fed-batch phase is performed as a second step b).

Specifically, the second step b) employs a feed medium in a fed-batch phase that provides for a supplemental carbon source in a growth limiting amount to keep the specific growth rate within the range of 0.0001 h⁻¹ to 0.2 h⁻¹, preferably less than or up to any of 0.2, 0.15, 0.1 or 0.15 h⁻¹.

According to a specific aspect, the invention further provides for a pat1 knockout eukaryotic host cell producing a protein of interest (POI) at an at least 1.1-fold or at least 1.2-fold increased yield and/or titer and/or specific productivity (μg/g cell dry mass (CDM), in particular yeast dry mass (YDM) for yeast cells) per hour and/or volumetric productivity (μg/L per hour) compared to the host cell without said pat1 knockout, preferably under carbon source-limiting conditions to produce said POI. Specifically, the increase is compared to the host cell without said pat1 knockout.

Specifically, the pat1 knockout cell as described herein is characterized by the genetic engineering of the host cell and the respective features of the host cell, as further described herein, with respect to the host cell of the invention which comprises a knockout of a pat1 polynucleotide or gene.

Specifically, the pat1 knockout cell is a eukaryotic host cell as described herein.

Specifically, the pat1 knockout cell described herein is characterized by one or more of the following features:
a) the product yield and/or titer, the specific productivity or volumetric productivity for said POI is at least 1.1-fold or at least 1.2-fold increased under carbon source limiting conditions to produce said POI;
b) the carbon-source limiting conditions are characterized by the presence of a growth-limiting amount of a carbon source in the host cell culture, which is up to 1 g/L;
c) the pat1 gene encodes a Pat1 protein that is Pat1 comprising SEQ ID NO:1, SEQ ID NO:3, or any one of SEQ ID NO:13 to 17, or SEQ ID NO:97-101, or a Pat1 ortholog to any one of the foregoing, preferably wherein the Pat1 is endogenous to the wild-type host cell;
d) the pat1 knockout host cell is a production host cell suitable for use in a recombinant host cell culture to produce the POI;
e) the pat1 knockout host cell is a yeast or mammalian host cell;
f) the POI is a heterologous peptide, polypeptide, protein or fusion protein, preferably wherein the POI is a secreted POI;
g) the POI is a therapeutic or diagnostic product; preferably wherein the POI is a therapeutic product selected from the group consisting of an antigen-binding protein, an enzyme, a peptide, a protein antibiotic, a toxin fusion protein, a structural protein, a regulatory protein, a protein vaccine antigen, a hormone, a growth factor, a cytokine, and a blood clotting or coagulation factor, preferably wherein the antigen-binding protein is an antibody molecule as further described herein;
h) the POI is a process enzyme or metabolic enzyme.

Specifically, the pat1 knockout cell as described herein is a P. pastoris cell, such as e.g., K. phaffii, K. pastoris or K. pseudopastoris, and the pat1 gene encodes a Pat1 protein comprising SEQ ID NO:1 or SEQ ID NO:3, or a Pat1 ortholog to the respective SEQ ID NO:1 or SEQ ID NO:3, as further described herein.

Specifically, the pat1 knockout cell as described herein is a CHO cell, and the pat1 gene encodes a Pat1 protein comprising any one of SEQ ID NO:13 to 17, or SEQ ID NO:97-101, or a Pat1 ortholog to the SEQ ID NO:13 to 17, as further described herein. Specifically, the Pat1 protein or Pat1 ortholog originates from a host cell selected from the group consisting of any one of Komagataella phaffii, Komagataella pastoris, Komagataella pseudopastoris, Saccharomyces cerevisiae, Yarrowia lipolytica, Ogataea polymorpha, Schizosaccharomyces pombe, Trichoderma reesei, Kluyveromyces lactis, Aspergillus niger, Homo sapiens, or Cricetulu griseus (in particular CHO) host cells.

Specifically,
a) the host cell is a Komagataella phaffii host cell, and the Pat1 protein comprises or consists of at least 90%, or at least 95%, or at least 96%, or at least 97%, or at least 98%, or at least 99%, or 100% sequence identity to SEQ ID NO:1; or
b) the host cell is a Komagataella pastoris host cell, and the Pat1 protein comprises or consists of at least 90%, or at least 95%, or at least 96%, or at least 97%, or at least 98%, or at least 99%, or 100% sequence identity to SEQ ID NO:3; or
c) the host cell is a Saccharomyces cerevisiae host cell, and the Pat1 protein comprises or consists of at least 90%, or at least 95%, or at least 96%, or at least 97%, or at least 98%, or at least 99%, or 100% sequence identity to SEQ ID NO:4; or
d) the host cell is a Yarrowia lipolytica host cell, and the Pat1 protein comprises or consists of at least 90%, or at least 95%, or at least 96%, or at least 97%, or at least 98%, or at least 99%, or 100% sequence identity to SEQ ID NO:5; or
e) the host cell is an Ogataea polymorpha host cell, and the Pat1 protein comprises or consists of at least 90%, or at least 95%, or at least 96%, or at least 97%, or at least 98%, or at least 99%, or 100% sequence identity to SEQ ID NO:6; or
f) the host cell is a Schizosaccharomyces pombe host cell, and the Pat1 protein comprises or consists of at least 90%, or at least 95%, or at least 96%, or at least 97%, or at least 98%, or at least 99%, or 100% sequence identity to SEQ ID NO:7; or
g) the host cell is a Trichoderma reesei host cell, and the Pat1 protein comprises or consists of at least 90%, or at least 95%, or at least 96%, or at least 97%, or at least 98%, or at least 99%, or 100% sequence identity to SEQ ID NO:8; or
h) the host cell is a Kluyveromyces lactis host cell, and the Pat1 protein comprises or consists of at least 90%, or at least 95%, or at least 96%, or at least 97%, or at least 98%, or at least 99%, or 100% sequence identity to SEQ ID NO:9; or
i) the host cell is an Aspergillus niger host cell, and the Pat1 protein comprises or consists of at least 90%, or at least 95%, or at least 96%, or at least 97%, or at least 98%, or at least 99%, or 100% sequence identity to SEQ ID NO:10.
j) the host cell is a Homo sapiens host cell, and the Pat1 protein comprises or consists of at least 90%, or at least 95%, or at least 96%, or at least 97%, or at least 98%, or at least 99%, or 100% sequence identity to SEQ ID NO:11 or 12; or
k) the host cell is a Cricetulu griseus host cell, and the Pat1 protein comprises or consists of at least 90%, or at least 95%, or at least 96%, or at least 97%, or at least 98%, or at least 99%, or 100% sequence identity to any one of SEQ ID NO:13 to 17, or any one of SEQ ID NO:97-101.

Specifically, the pat1 knockout host cell is a CHO cell, such as further described herein.

Specifically, the pat1 knockout host cell is a CHO cell and the Pat1 protein comprises or consists of at least 90%, or at least 95%, or at least 96%, or at least 97%, or at least 98%, or at least 99%, or 100% sequence identity to any one of SEQ ID NO:13 to 17, or any one of SEQ ID NO:97-101.

Specifically, the pat1 knockout eukaryotic host cell is a host cell that is characterized or produced as further described herein.

### FIGURES

FIG. 1 shows sequences referred to herein.

FIG. 2 shows cB72.3 and Cas9 vector used for creating reporter cell lines in the CHOK1SV host. A: The cB72.3 vector contains a transcription unit incorporating the cB72.3 light and heavy chain genes driven by the promoter of the murine CMV intermediate early gene 1 (mCMV) and its first intron (Intron A) and the flanking exons encoding the 5′UTR. Glutamine synthetase cDNA (GS) driven by SV40E promoter and is used as a selection marker. B: The Cas9 vector contains a transcriptional unit incorporating human codon optimized Streptococcus pyogenes Cas9 driven by the EF-1a core promoter. Selection using Blasticidin resistance marker (BlastR).

FIG. 3 is a schematic of the Cricetulus griseus PATL1 gene. The 17 exons are annotated. CRISPR gRNA positioning on the exons 4, 12 and 14 (guides highlighted: Patl1_1, Patl1_2, Patl1_3) are highlighted.

FIG. 4 shows the data from illumina sequencing of PCR amplicons derived from CRISPR gRNA transfected pools. Genomic DNA was extracted 3-days post transfection. PCR amplification of the target locus (ex 4,12 and 14 of PATL1) was followed by illumina sequencing. Sequencing reads were analysed using CRISPResso (version 1) software and the proportion of reads containing insertions, deletions and substitutions (indels) are presented. Data are mean±st dev, n=2. Pools transfected with gRNA targeting the PATL1 gene had >80% of sequencing reads containing indels. Non targeting, no-guide and untreated showed minimal indels.

FIG. 5: shows secreted cB72.3 mAb concentration of transfected pools (ex 4,12 and 14 of PATL1) determined by Sartorius Octet® using Protein A biosensors 9 days post transfection. Data are mean±st dev, n=4. PATL1 KO exhibited high levels of mAb compared to controls (Non-targeting, No guide and parental/mock).

FIG. 6 summarizes growth and productivity measurements from 10-day culture of transfected pools expressing cB72.3. A: integral of viable cell concentration (IVCC). B: harvest cB72.3 at day 10 determined by Sartorius Octet® using Protein A biosensors. Data are mean±Standard deviation (n=4). PATL1 KO increased titers by >30% over controls. Standard deviation plotted from four replicates.

FIG. 7 summarises PATL1 mRNA fold change in transfected pools relative to parent host cells. qPCR was completed using the 2-AACT with Glyceraldehyde 3-phosphate dehydrogenase (GAPDH) as the housekeeping gene. Data are presented relative to the parental controls. Data are mean (n=3)±Standard deviation. PATL1 KO pool showed >80% reduction in transcript level compared to controls. Standard deviation plotted from three replicates.

FIG. 8: summarizes growth and productivity measurements from 10-day culture of clones expressing cB72.3. All the clones were tested by qPCR for functional KO of the PATL 1 gene. (A) IVCC of all clones. Data are mean (n=2)±Standard deviation. (B) Titre levels of KO clones and controls (determined by Sartorius Octet® using Protein A biosensors and ranked in the order based on Titre). Data are mean (n=2)±Standard deviation. C) qPCR assay was performed on KO clones showing reduction in transcript level for PATL1 gene. Transcript amounts are presented relative to non-targeting control. Data are mean (n=3)±Standard deviation.

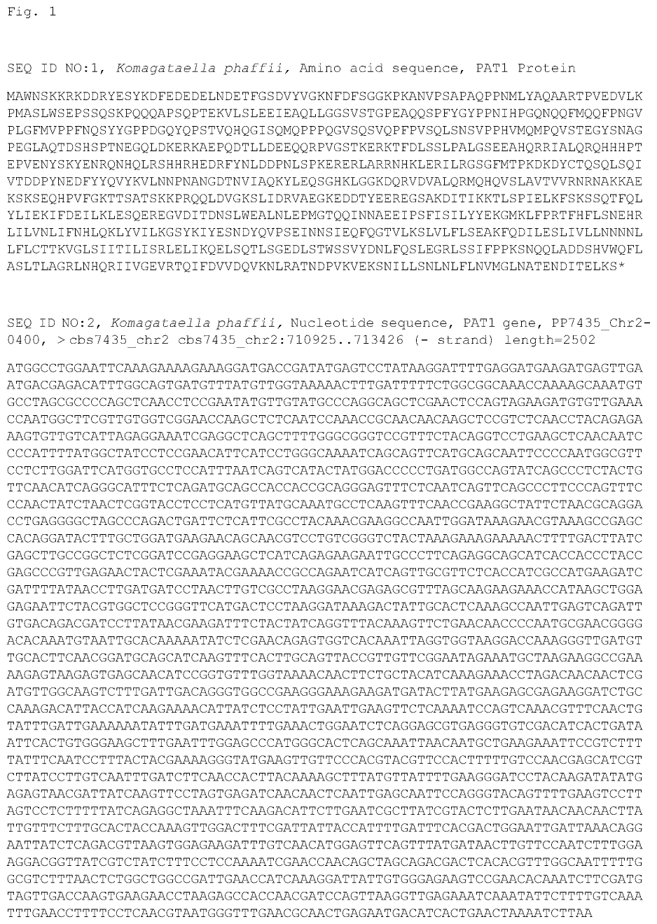

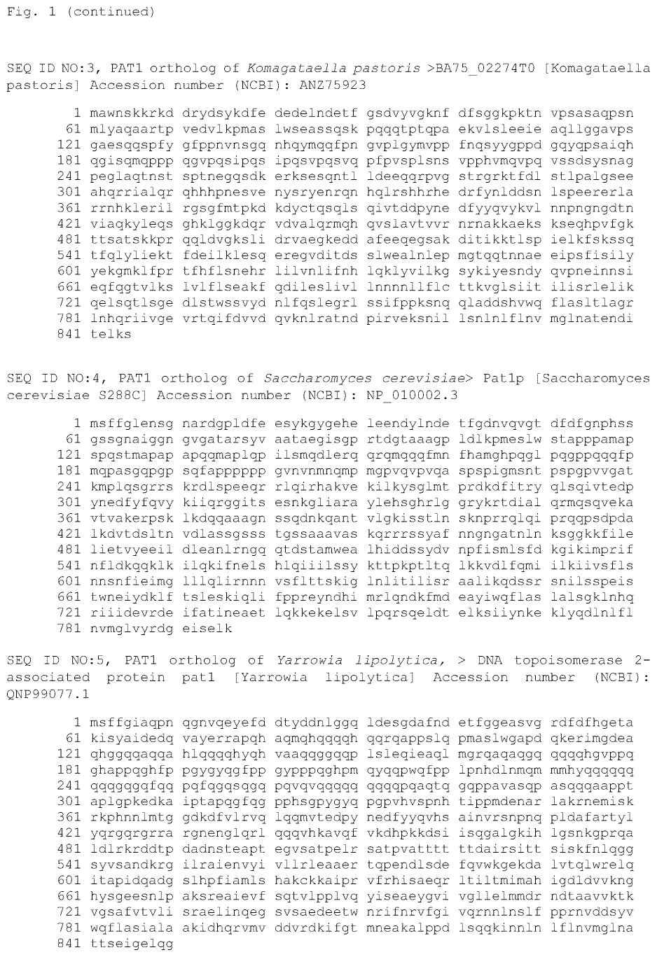

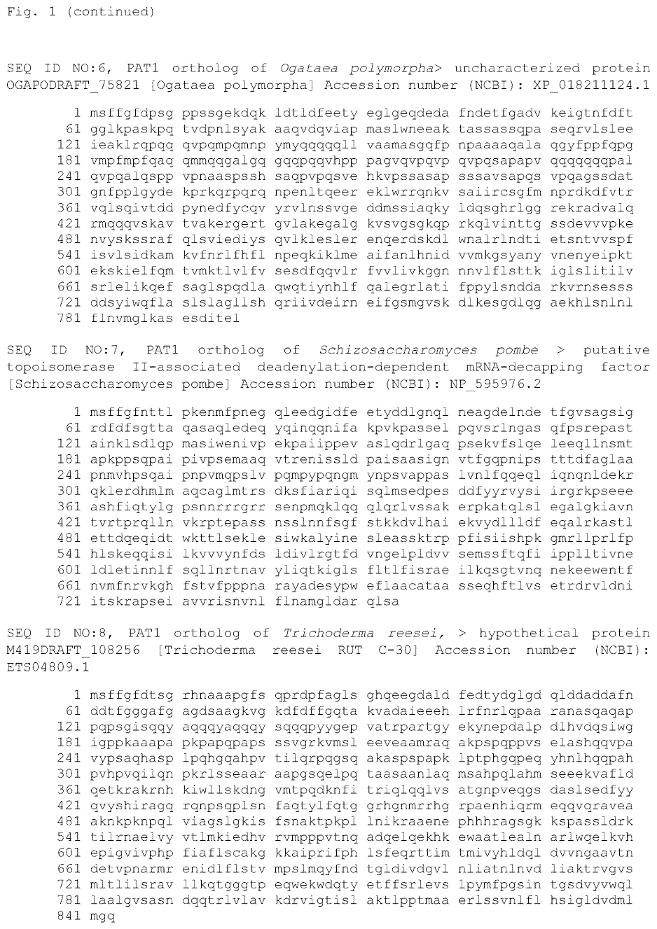

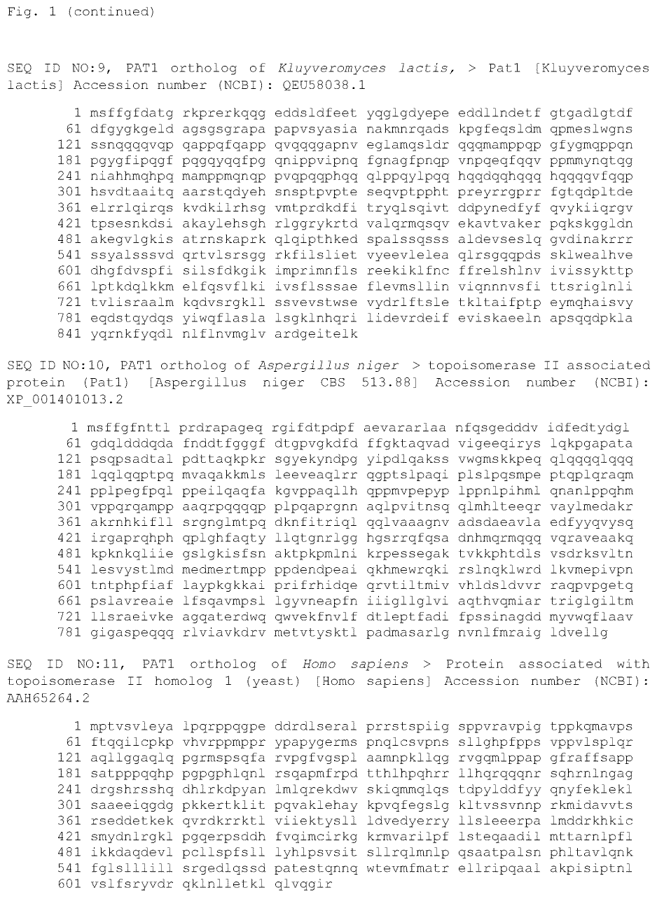

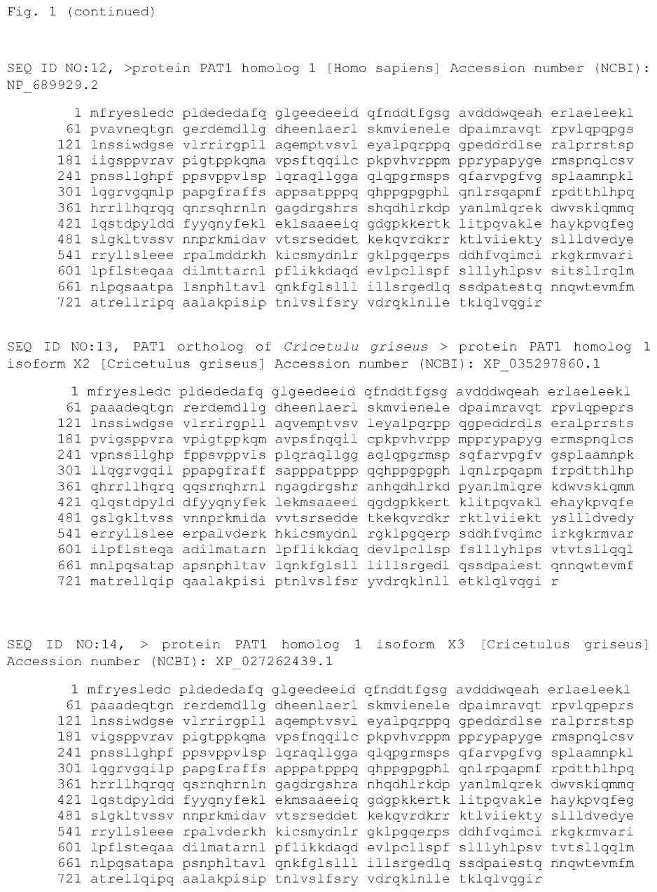

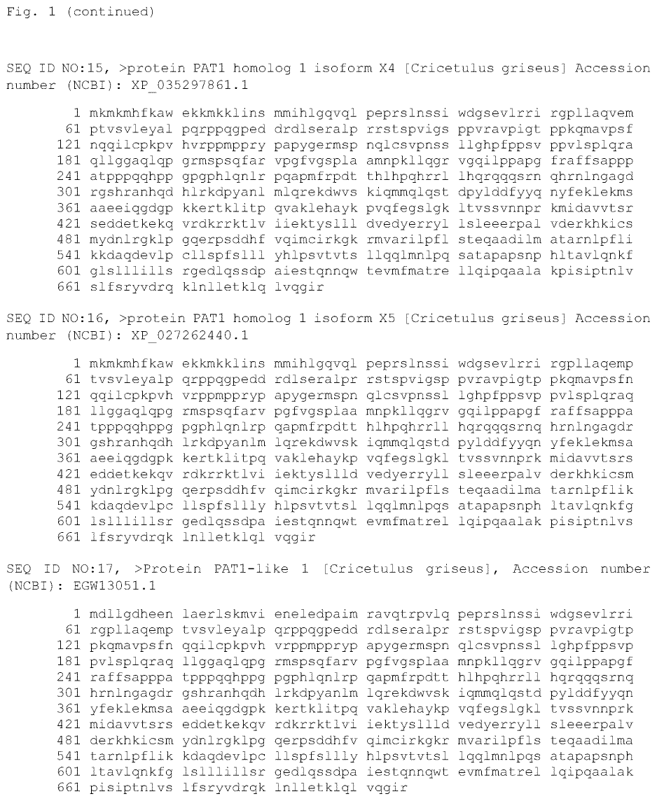

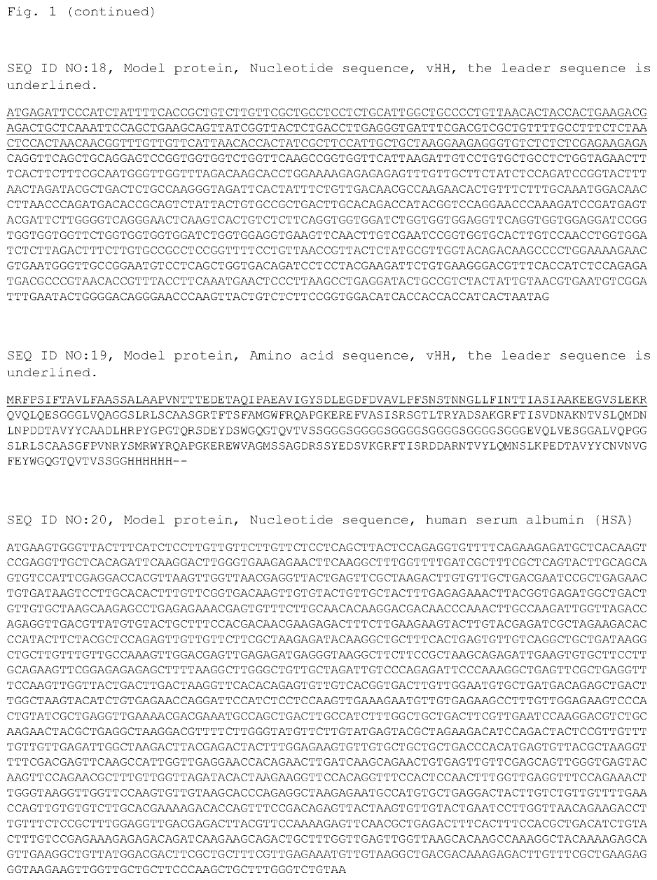

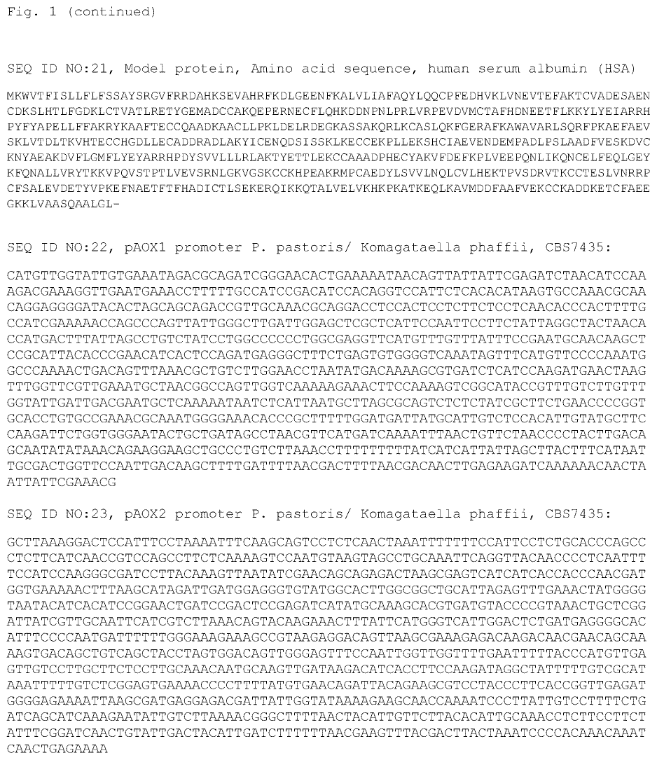

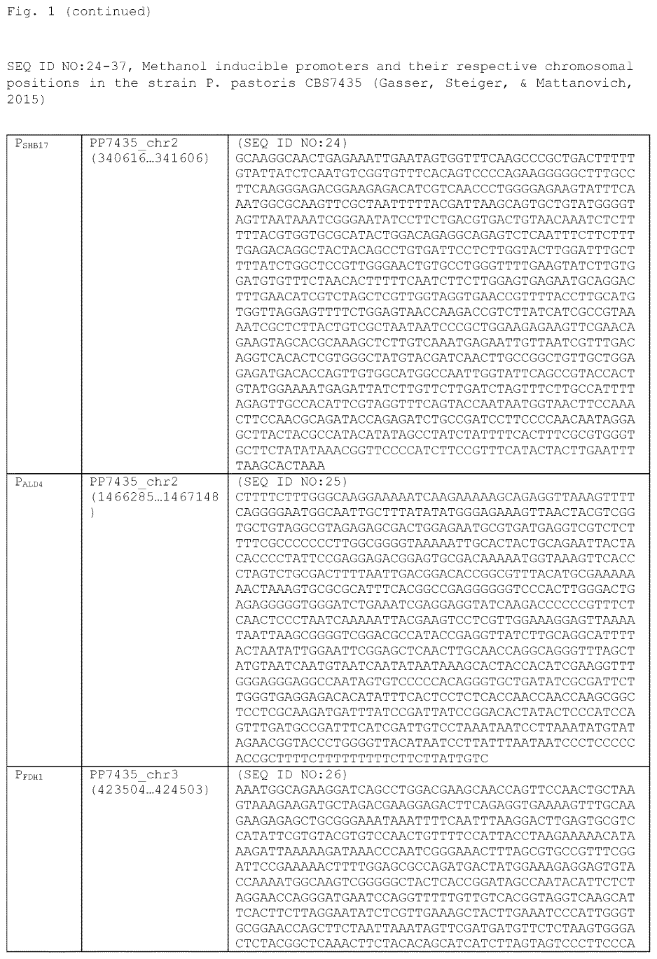

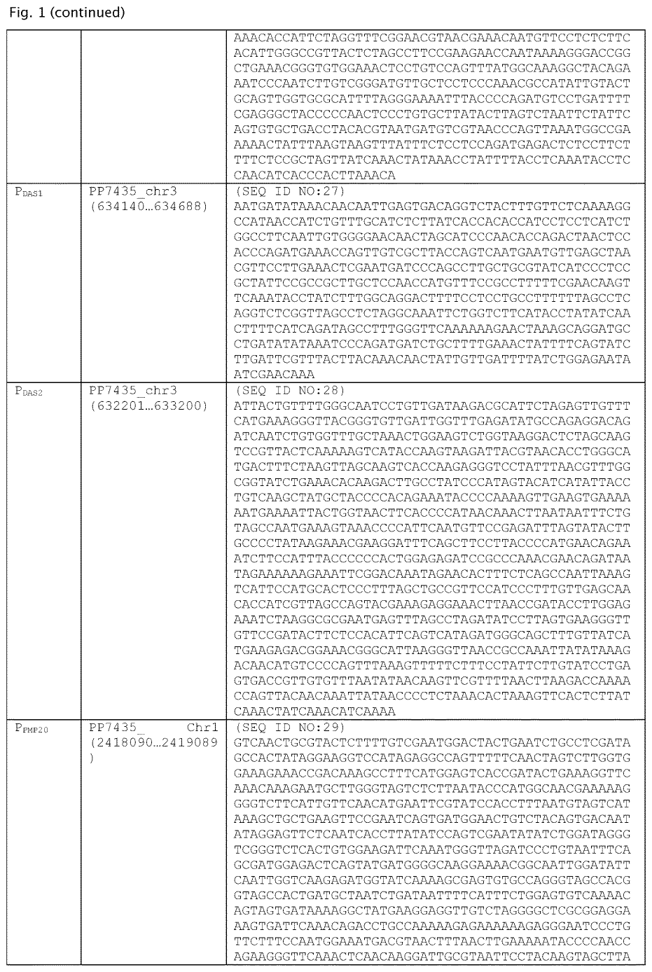

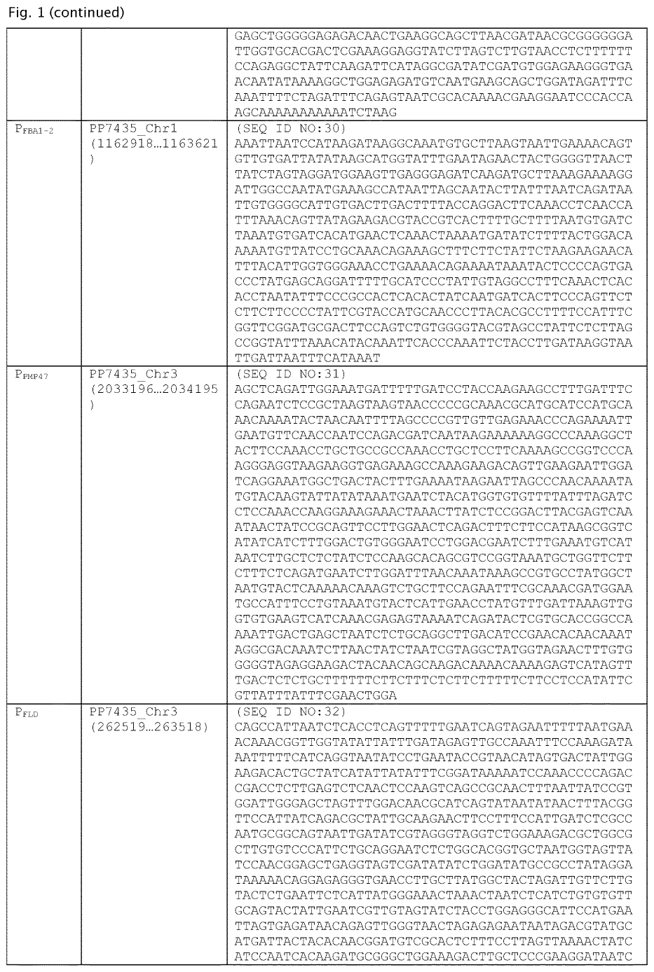

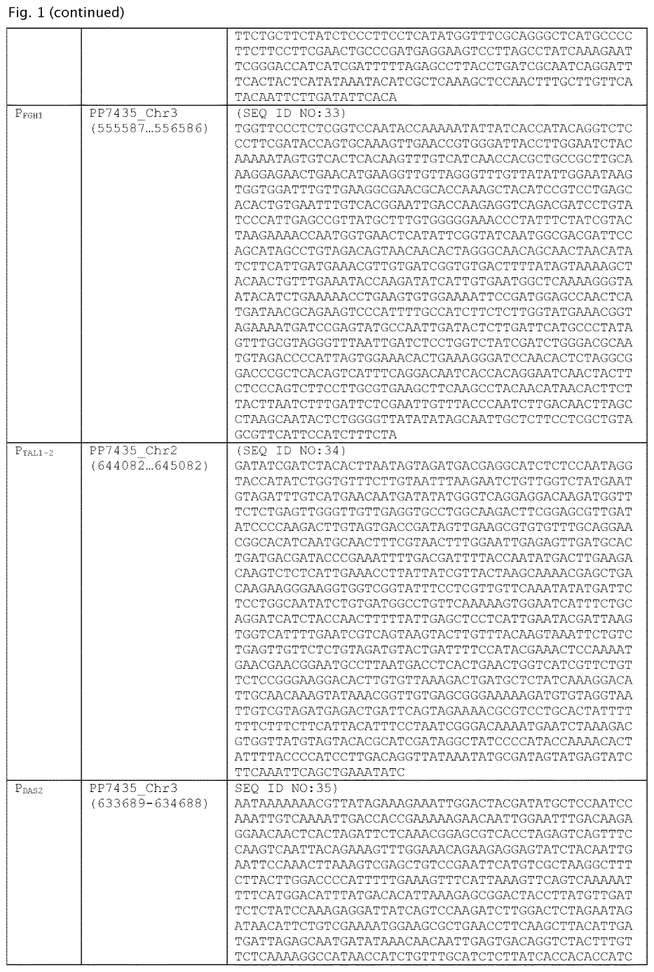

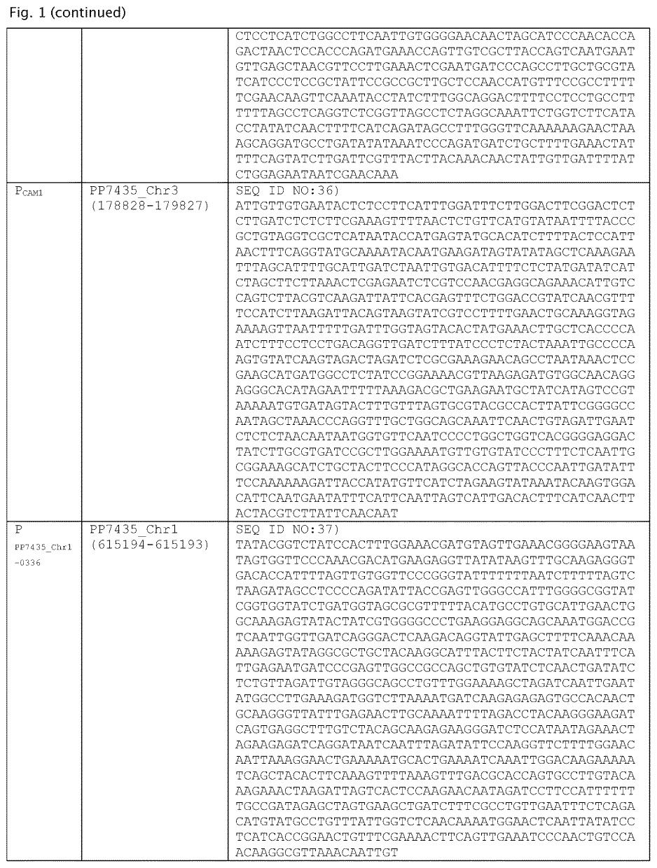

### DETAILED DESCRIPTION

Unless indicated or defined otherwise, all terms used herein have their usual meaning in the art, which will be clear to the skilled person. Reference is for example made to the standard handbooks, such as Sambrook et al., 2012, Molecular Cloning: A Laboratory Manual, volumes 1-4, Cold Spring Harbor Press, NY); Lewin, “Genes IV”, Oxford University Press, New York, (1990), and Janeway et al., “Immunobiology” (5th Ed., or more recent editions), Garland Science, New York, 2001, Ausubel et al., Current Protocols in Molecular Biology, John Wiley and Sons, Baltimore, Md. (1989), Vega et al., Gene Targeting, CRC Press, Ann Arbor Mich. (1995), and Vectors: A Survey of Molecular Cloning Vectors and Their Uses, Butterworths, Boston Mass. (1988).

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

Specific terms as used throughout the specification have the following meaning.

The term “carbon source” also referred as “carbon substrate” as used herein shall mean a fermentable carbon substrate, typically a source carbohydrate, suitable as an energy source for microorganisms, such as those capable of being metabolized by host organisms or production cell lines, in particular sources selected from the group consisting of monosaccharides, oligosaccharides, polysaccharides, alcohols including glycerol, in the purified form, in minimal media or provided in raw materials, such as a complex nutrient material. The carbon source may be used as described herein as a single carbon source or as a mixture of different carbon sources.

A non-methanol carbon source is herein understood as a carbon source which is any other than methanol, in particular a methanol-free carbon source.

A “basal carbon source” such as used as described herein typically is a carbon source suitable for cell growth, such as a nutrient for host cells, in particular for eukaryotic cells. The basal carbon source may be provided in a medium, such as a basal medium or complex medium, but also in a chemically defined medium containing a purified carbon source. The basal carbon source typically is provided in an amount to provide for cell growth, in particular during the growth phase in a cultivation process, for example to obtain cell densities of at least 5 g/L cell dry mass, preferably at least 10 g/L cell dry mass, or at least 15 g/L cell dry mass e.g., exhibiting viabilities of more than 90% during standard sub-culture steps, preferably more than 95%.

The basal carbon source is typically used in an excess or surplus amount, which is understood as an excess providing energy to increase the biomass e.g., during the cultivation of a cell line with a high specific growth rate, such as during the growth phase of a cell line in a batch or fed-batch cultivation process. This surplus amount is particularly in excess of the limited amount of a supplemental carbon source (as used under growth-limited conditions) to achieve a residual concentration in the fermentation broth that is measurable and typically at least 10-fold higher, preferably at least 50-fold or at least 100-fold higher than during feeding the limited amount of the supplemental carbon source.

A “supplemental carbon source” such as described herein typically is a supplemental substrate facilitating the production of fermentation products by production cell lines, in particular in the production phase of a cultivation process. The production phase specifically follows a growth phase e.g., in batch, fed-batch and continuous cultivation process. The supplemental carbon source specifically may be contained in the feed of a fed-batch process. The supplemental carbon source is typically employed in a cell culture under carbon substrate limited conditions i.e., using the carbon source in a limited amount.

The term “limited amount” of a carbon source, a “limited carbon source” or “carbon source limitation” is herein understood to specifically refer to the type and amount of a carbon substrate facilitating the production of fermentation products by production cell lines, in particular in a cultivation process with controlled growth rates of less than the maximum growth rate. The production phase specifically follows a growth phase e.g., in batch, semi fed-batch, fed-batch, perfusion and/or and continuous cultivation process. Cell culture processes may employ batch culture, semi fed-batch, perfusion culture, continuous culture, and fed-batch culture. Specifically, a bioreactor is used which is suitable for any such cell culture or respective fermentation processes.

Batch culture is a culture process by which a small amount of a seed culture solution is added to a medium and cells are grown without adding an additional medium or discharging a culture solution during culture. Continuous culture is a culture process by which a medium is continuously added and discharged during culture. The continuous culture also includes perfusion culture. Fed-batch culture, which is an intermediate between the batch culture and the continuous culture and also referred to as semi-batch culture, is a culture process by which a medium is continuously or sequentially added during culture but, unlike the continuous culture, a culture solution is not continuously discharged.

Specifically preferred is a fed-batch process which is based on feeding of a growth limiting nutrient substrate to a culture. The fed-batch strategy, including single fed-batch or repeated fed-batch fermentation, is typically used in bio-industrial processes to reach a high cell density in the bioreactor.

The host cell culture is suitably performed in a bioreactor or bioreactor unit, which is particularly understood as a fermenter or fermentation unit, or any other reaction vessel and production unit comprising a reaction vessel. Specifically, a bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO₂ levels, maintenance of pH level, agitation (e.g., stirring), and/or cleaning/sterilizing.

The controlled addition of the carbon substrate directly affects the growth rate of the culture and helps to avoid overflow metabolism or the formation of unwanted metabolic byproducts. Under carbon source limited conditions, the carbon source specifically may be contained in the feed of a fed-batch process. Thereby, the carbon substrate is provided in a limited amount.

In chemostat or continuous culture as described herein, the growth rate can be tightly controlled.

The limited amount of a carbon source is herein particularly understood as the amount of a carbon source necessary to keep a production cell line under growth-limited conditions e.g., in a production phase or production mode. Such a limited amount may be employed in a fed-batch process, where the carbon source is contained in a feed medium and supplied to the culture at low feed rates for sustained energy delivery e.g., to produce a POI, while keeping the biomass at low specific growth rates. A feed medium is typically added to a fermentation broth during the production phase of a cell culture.

The limited amount of a carbon source may, for example, be determined by the residual amount of the carbon source in the cell culture broth, which is below a predetermined threshold or even below the detection limit as measured in a standard (carbohydrate) assay. The residual amount typically would be determined in the fermentation broth upon harvesting a fermentation product.

The limited amount of a carbon source may as well be determined by defining the average feed rate of the carbon source to the fermenter e.g., as determined by the amount added over the full cultivation process e.g., the fed-batch phase, per cultivation time, to determine a calculated average amount per time. This average feed rate is kept low to ensure complete usage of the supplemental carbon source by the cell culture, e.g., between 0.6 g L⁻¹ h⁻¹ (g carbon source per L initial fermentation volume and h time) and 25 g L⁻¹ h⁻¹, preferably between 1.6 g L⁻¹ h⁻¹ and 20 g L⁻¹ h⁻¹.

The limited amount of a carbon source may also be determined by measuring the specific growth rate, which specific growth rate is kept low e.g., lower than the maximum specific growth rate, during the production phase e.g., within a predetermined range, such as in the range of 0.001 h⁻¹ to 0.20 h⁻¹, or 0.005 h⁻¹ to 0.20 h⁻¹, preferably between 0.01 h⁻¹ and 0.15 h⁻¹.

The term “carbon source limitation” as used herein is specifically not used for conditions of carbon source-starvation.

The term “carbon source-starvation” is herein understood to specifically refer to cultivation in media without any carbon source or after complete consumption of a carbon source. Carbon-source starvation can e.g., be determined by growth rate measurements or by quantification of carbon-source molecules in the media and culture supernatant (e.g., HPLC)

Specifically, a feed medium is used which is chemically defined and methanol-free.

The term “chemically defined” with respect to cell culture medium, such as a minimal medium or feed medium in a fed-batch process, shall mean a cultivation medium suitable for the in vitro cell culture of a production cell line, in which all of the chemical components and (poly)peptides are known. Typically, a chemically defined medium is entirely free of animal-derived components and represents a pure and consistent cell culture environment.

The term “host cell” as used herein shall refer to a single cell, a single cell clone, or a cell line of a host cell.

The term “cell” with respect to a “host cell” as used herein shall refer to a single cell, a single cell clone, or a cell line of a host cell.

The term “cell line” as used herein refers to an established clone of a particular cell type that has acquired the ability to proliferate over a prolonged period of time. A cell line is typically used for expressing an endogenous or recombinant nucleic acid molecule or gene, or products of a metabolic pathway to produce polypeptides or cell metabolites mediated by such polypeptides. A “production host cell line” or “production cell line” is commonly understood to be a cell line ready-to-use for cell culture in a bioreactor to obtain the product of a production process, such as a POI.

The term “host cell” as used herein shall particularly apply to any cell, which is suitably used for recombination purposes to produce a POI or a host cell metabolite. It is well understood that the term “host cell” does not include human beings. Specifically, recombinant host cells as described herein are artificial organisms and derivatives of native (wild-type) host cells. It is well understood that the host cells, methods and uses described herein, e.g., specifically referring to those comprising one or more genetic modifications, heterologous expression cassettes or artificial expression constructs, said transfected or transformed host cells and recombinant proteins, are non-naturally occurring, are “man-made” or synthetic, and are therefore not considered as a result of “law of nature”. Genetic modifications described herein may employ tools, methods and techniques known in the art, such as described by Sambrook et al., 2012, Molecular Cloning: A Laboratory Manual, volumes 1-4, Cold Spring Harbor Press, NY).

The term “cell culture” or “culturing” or “cultivation” as used herein with respect to a host cell refers to the maintenance of cells in an artificial, e.g., an in vitro environment, under conditions favoring growth, differentiation or continued viability, in an active or quiescent state, of the cells, specifically in a controlled bioreactor according to methods known in the industry.

When culturing a cell culture using appropriate culture media, the cells are brought into contact with the media in a culture vessel or with substrate under conditions suitable to support culturing the cells in the cell culture. Standard cell culture media and techniques are well-known in the art.

The cell cultures as described herein particularly employ techniques which provide for the production of a secreted POI, such as to obtain the POI in the cell culture medium, which is separable from the cellular biomass, herein referred to as “cell culture supernatant”, and may be purified to obtain the POI at a higher degree of purity.

Cell culture media provide the nutrients necessary to maintain and grow cells in a controlled, artificial and in vitro environment. Characteristics and compositions of the cell culture media vary depending on the particular cellular requirements. Important parameters include osmolality, pH, and nutrient formulations. Feeding of nutrients may be done in a continuous or discontinuous mode according to methods known in the art.

Whereas a batch process is a cell culture mode in which all the nutrients necessary for culturing the cells are contained in the initial culture medium, without additional supply of further nutrients during fermentation, in a fed-batch or continuous process, after a batch phase, a feeding phase takes place in which one or more nutrients are supplied to the culture by feeding. Although in most processes the mode of feeding is critical and important, the host cell and methods described herein are not restricted with regard to a certain mode of cell culture.

A POI can be produced using the host cell and the respective cell line described herein, by culturing in an appropriate medium, isolating the expressed product or metabolite from the culture, and optionally purifying it by a suitable method.

Methods for recovering and/or purifying a POI are well established in the art. Specifically, a physical or chemical or physical-chemical method is used. The physical or chemical or physical-chemical method can be a filtering method, a centrifugation method, an ultracentrifugation method, an extraction method, a lyophilization method, a precipitation method, a chromatography method or a combination of two or more of any such methods. Specifically, the chromatography method comprises one or more of size-exclusion chromatography (or gel filtration), ion exchange chromatography, e.g., anion or cation exchange chromatography, affinity chromatography, hydrophobic interaction chromatography, and/or multimodal chromatography.

Several different approaches for the production of the POI as described herein are preferred. A POI may be expressed, processed and optionally secreted by transforming or transfecting a host cell with an expression vector harboring recombinant DNA encoding the relevant protein, preparing a culture of the transformed or transfected cell, growing the culture, inducing transcription and POI production, and recovering the POI.

In certain embodiments, the cell culture process is a fed-batch process. Specifically, a host cell transfected with a nucleic acid construct encoding a desired recombinant POI, is cultured in a growth phase and transitioned to a production phase in order to produce a desired recombinant POI.

In another embodiment, host cells described herein are cultured in a continuous mode e.g., employing a chemostat. A continuous fermentation process is characterized by a defined, constant and continuous rate of feeding of fresh culture medium into a bioreactor, whereby culture broth is at the same time removed from the bioreactor at the same defined, constant and continuous removal rate. By keeping culture medium, feeding rate and removal rate at the same constant level, the cell culture parameters and conditions in the bioreactor remain constant.

In another embodiment, host cells described herein are cultured in a perfusion mode e.g., culturing cells within a device while supplying fresh medium and removing the supernatant.

A stable cell culture as described herein is specifically understood to refer to a cell culture maintaining the genetic properties, specifically keeping the POI production level high e.g., at least at a μg level, even after about 20 generations of cultivation, preferably at least 30 generations, more preferably at least 40 generations, most preferred of at least 50 generations. Specifically, a stable recombinant host cell line is provided which is considered a great advantage when used for industrial scale production. Specifically, the host cell is suitable for a cell culture in a bioreactor, or is capable of being cultured or grown in a bioreactor.

The cell culture described herein is particularly advantageous for use in a method of POI production on an industrial manufacturing scale e.g., with respect to both the volume and the technical system, in combination with a cultivation mode that is based on feeding of nutrients, in particular a fed-batch or batch process, or a continuous or semi-continuous process (e.g., chemostat).

The host cell described herein is typically tested for its capacity to express the GOI for POI production, tested for the POI yield and/or titer by any of the following tests: ELISA, activity assay, capillary electrophoresis, HPLC, or other suitable tests, such as SDS-PAGE and Western Blotting techniques, or mass spectrometry.

To determine the effect of a genetic modification on the expression control or reduction of a gene encoding the Pat1 protein as described herein in the respective cell culture and e.g., on its effect on POI production, the host cell line may be cultured in microtiter plates, shake flask, or bioreactor using fed-batch or chemostat fermentations in comparison with strains without such genetic modification in the respective cell.

The production method described herein specifically allows for the fermentation on a pilot or industrial scale. The industrial process scale would preferably employ volumes of at least 10 L, specifically at least 50 L, preferably at least 1 m³, preferably at least 10 m³, most preferably at least 100 m3.

Production conditions in industrial scale are preferred, which refer to e.g., fed batch culture in reactor volumes of 100 L to 10 m³ or larger, employing typical process times of several days, or continuous processes in fermenter volumes of approximately 50-1000 L or larger, with dilution rates of approximately 0.001-0.15 h⁻¹.

The devices, facilities and methods used for the purpose described herein are specifically suitable for use in and with culturing any desired cell line. Further, the devices, facilities and methods are suitable for culturing any eukaryotic host cell type, and are particularly suitable for production operations configured for production of pharmaceutical and biopharmaceutical products, such as polypeptide or protein products (POI), nucleic acid products (for example DNA or RNA), or cells and/or viruses such as those used in cellular and/or viral therapies. Unless stated otherwise herein, the devices, facilities, and methods can include any desired volume or production capacity including but not limited to bench-scale, pilot-scale, and full production scale capacities.

Moreover, the devices, facilities, and methods can include any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter.” For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation process. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally, and unless otherwise stated, the devices, systems, and methods described herein can be housed and/or performed in a single location or facility or alternatively be housed and/or performed at separate or multiple locations and/or facilities.

Suitable techniques may encompass culturing in a bioreactor starting with a batch phase, followed by a short exponential fed batch phase at high specific growth rate, further followed by a fed batch phase at a low specific growth rate. Another suitable culture technique may encompass a batch phase followed by a fed-batch phase at any suitable specific growth rate or combinations of specific growth rates such as going from high to low growth rate over POI production time, or from low to high growth rate over POI production time. Another suitable culture technique may encompass a batch phase followed by a continuous culturing phase at a low dilution rate.

A preferred embodiment includes a batch culture to provide biomass followed by a fed-batch culture for high yield POI production.

It is preferred to culture a host cell as described herein in a bioreactor under growth conditions to obtain a cell density of at least 1 g/L cell dry weight, more preferably at least 10 g/L cell dry weight, preferably at least 20 g/L cell dry weight, preferably at least any one of 30, 40, 50, 60, 70, or 80 g/L cell dry weight. It is advantageous to provide for such yields of biomass production on a pilot or industrial scale.

A growth medium allowing the accumulation of biomass, specifically a basal growth medium, typically comprises a carbon source, a nitrogen source, a source for sulphur and a source for phosphate. Typically, such a medium comprises furthermore trace elements and vitamins, and may further comprise amino acids, peptone or yeast extract.

Preferred nitrogen sources include NH₄H₂PO₄, or NH₃ or (NH₄)₂SO₄;

Preferred sulphur sources include MgSO₄, or (NH₄)₂SO₄ or K₂SO₄; Preferred phosphate sources include NH₄H₂PO₄, or H₃PO₄, or NaH₂PO₄, KH₂PO₄, Na₂HPO₄ or K₂HPO₄;

Further typical medium components include KCl, CaCl₂), and Trace elements such as: Fe, Co, Cu, Ni, Zn, Mo, Mn, I, B;

Preferably the medium is supplemented with vitamins essential for growth, e.g., B vitamins such as B7;

A typical growth medium for P. pastoris comprises glycerol, sorbitol or glucose, NH₄H₂PO₄, MgSO₄, KCl, CaCl₂), biotin, and trace elements.

In the production phase a production medium is specifically used with only a limited amount of a supplemental carbon source.

Preferably, the host cell line is cultured in a mineral medium with a suitable carbon source, thereby further simplifying the isolation process significantly. An example of a preferred mineral medium is one containing a utilizable carbon source (e.g., glucose, glycerol, sorbitol, methanol, ethanol, or combinations thereof), salts containing the macro elements (potassium, magnesium, calcium, ammonium, chloride, sulphate, phosphate) and trace elements (copper, iodide, manganese, molybdate, cobalt, zinc, and iron salts, and boric acid), and optionally vitamins or amino acids, e.g., to complement auxotrophies.

Specifically, the cells are cultured under conditions suitable to effect expression of the desired POI, which can be purified from the cells or culture medium, depending on the nature of the expression system and the expressed protein, e.g., whether the protein is fused to a signal peptide and whether the protein is soluble or membrane-bound. As will be understood by the skilled artisan, culture conditions will vary according to factors that include the type of host cell and particular expression vector employed.

A typical production medium comprises a supplemental carbon source, and further NH₄H₂PO₄, MgSO₄, KCl, CaCl₂), biotin, and trace elements.

For example, the feed of the supplemental carbon source added to the fermentation may comprise a carbon source with up to 50 wt % utilizable sugars, or up to 100% utilizable alcohols.

The fermentation preferably is carried out at a pH ranging from 3 to 8.

Typical fermentation times are about 24 to 120 hours with temperatures in the range of 20° C. to 35° C., preferably 22-30° C.

The POI is preferably expressed employing conditions to produce yields of at least 1 mg/L, preferably at least 10 mg/L, preferably at least 100 mg/L, most preferred at least 1 g/L.

The term “expression” or “expression cassette” is herein understood to refer to nucleic acid molecules (herein also referred to as polynucleotides), which contain a desired coding sequence (herein referred to as a gene), and control sequences in operable linkage, so that hosts transformed or transfected with these molecules incorporate the respective sequences and are capable of producing the encoded proteins or host cell metabolites. The term “expression” as used herein refers to expression of a polynucleotide or gene, or to the expression of the respective polypeptide or protein.

One or more expression cassettes are herein also understood as “expression system”. The expression system may be included in an expression construct, such as a vector; however, the relevant DNA may also be integrated into a host cell chromosome. Expression may refer to secreted or non-secreted expression products, including polypeptides or metabolites.

Expression cassettes are conveniently provided as expression constructs e.g., in the form of “vectors” or “plasmids”, which are typically DNA sequences that are required for the transcription of cloned recombinant nucleotide sequences i.e., of recombinant genes and the translation of their mRNA in a suitable host organism. Expression vectors or plasmids usually comprise an origin for autonomous replication or a locus for genome integration in the host cells, selectable markers (e.g., an amino acid synthesis gene or a gene conferring resistance to antibiotics such as zeocin, kanamycin, G418 or hygromycin, nourseothricin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences, such as artificial chromosomes e.g., a yeast artificial chromosome (YAC).

Expression vectors may include but are not limited to cloning vectors, modified cloning vectors and specifically designed plasmids. Preferred expression vectors described herein are expression vectors suitable for expressing of a recombinant gene in a eukaryotic host cell and are selected depending on the host organism. Appropriate expression vectors typically comprise regulatory sequences suitable for expressing DNA encoding a POI in a eukaryotic host cell. Examples of regulatory sequences include promoter, operators, enhancers, ribosomal binding sites, and sequences that control transcription and translation initiation and termination. The regulatory sequences are typically operably linked to the DNA sequence to be expressed.

To allow expression of a recombinant nucleotide sequence in a host cell, a promoter sequence is typically regulating and initiating transcription of the downstream nucleotide sequence, with which it is operably linked. An expression cassette or vector typically comprises a promoter nucleotide sequence which is adjacent to the 5′ end of a coding sequence, e.g., upstream from and adjacent to the coding sequence (e.g., encoding a helper factor) or gene of interest (GOI), or if a signal or leader sequence is used, upstream from and adjacent to said signal and leader sequence, respectively, to facilitate translation initiation and expression of coding sequences to obtain the expression product (e.g., the POI).

Specific expression constructs described herein comprise a promoter operably linked to a nucleotide sequence encoding a POI under the transcriptional control of said promoter. Specifically, the promoter can be used which is not natively associated with said coding sequence.

Specific expression constructs described herein comprise a polynucleotide encoding the POI linked with a leader sequence (e.g., a secretion signal peptide sequence (pre-sequence), or a pro-sequence), which causes transport of the POI into the secretory pathway and/or secretion of the POI from the host cell. The presence of such a secretion leader sequence in the expression vector is typically required when the POI intended for recombinant expression and secretion is a protein which is not naturally secreted and therefore lacks a natural secretion leader sequence, or its nucleotide sequence has been cloned without its natural secretion leader sequence. In general, any secretion leader sequence effective to cause secretion of the POI from the host cell may be used. The secretion leader sequence may originate from yeast source e.g., from yeast alpha-factor such as MFa of Saccharomyces cerevisiae, or yeast phosphatase, from mammalian or plant source, or others.

In specific embodiments, multicloning vectors may be used, which are vectors having a multicloning site. Specifically, a desired heterologous polynucleotide can be integrated or incorporated at a multicloning site to prepare an expression vector. In the case of multicloning vectors, a promoter is typically placed upstream of the multicloning site.

The term “gene expression”, or “expressing a polynucleotide” or “expressing a nucleic acid molecule” as used herein, is meant to encompass at least one step selected from the group consisting of DNA transcription into mRNA, mRNA translation and processing, mRNA maturation, mRNA export, protein folding and/or protein transport.

The terms “control expression” or “reduce expression” typically refer to “underexpressing” and refer to any amount less than an expression level exhibited by a reference standard, which is the host cell prior to the engineering to reduce expression of a certain polynucleotide, or which is otherwise expressed in a host cell of the same type or species which is not engineered to lower expression of said polynucleotide. Reduction of expression as described herein specifically refers to a polynucleotide or gene encoding a defined Pat1 protein, in particular a gene that is endogenous to the host cell prior to engineering. In particular, the respective gene product is the defined Pat1 protein as described herein. Upon engineering the host cell by genetic modification to reduce expression of said gene the expression of said gene product or polypeptide is at a level which is less than the expression of the same gene product or polypeptide prior to a genetic modification of the host cell or in a comparable host which has not been genetically modified. “Less than” includes e.g., an amount that is reduced by a certain percentage e.g., a percentage of at least 10%, 20%, 30%, 40%, 50%, 60%, 70%, 80, 90% or higher. No expression of the gene product or a polypeptide is also encompassed by the term “reduction of expression” or “underexpression.”

According to specific embodiments described herein, the host cell is engineered to knock-down or knockout (for inactivation or deletion of a gene or a part thereof) the endogenous host cell gene encoding the Pat1 protein (as defined herein, including e.g., the respective homologue or orthologue), or other (coding or non-coding) nucleotide sequences which confer the host cell's ability to express or produce said Pat1 protein.

Specifically, a deletion strain is provided, wherein a nucleotide sequence is disrupted.

The term “disrupt” as used herein refers to the significant reduction to complete removal of the expression of one or more endogenous proteins in a host cell, such as by knock-down or knockout. This may be measured as presence of such one or more endogenous proteins in a cell culture or culture medium of the host cell, such as by mass spectrometry wherein the total content of a endogenous protein may be less than a threshold or non-detectable.

The term “disrupted” specifically refers to a result of genetic engineering by at least one step selected from the group consisting of gene silencing, gene knock-down, gene knockout, delivery of a dominant negative construct, conditional gene knockout, and/or by gene alteration with respect to a specific gene.

The term “knock-down”, “reduction” or “depletion” in the context of gene expression as used herein refers to experimental approaches leading to reduced expression of a given gene compared to expression in a control cell. Knock-down of a gene can be achieved by various experimental means such as introducing nucleic acid molecules into the cell which hybridize with parts of the gene's mRNA leading to its degradation (e.g., shRNAs, RNAi, miRNAs) or altering the sequence of the gene in a way that leads to reduced transcription, reduced mRNA stability or diminished mRNA translation.

The present disclosure, in part, is directed to methods of manufacturing a product, e.g., a recombinant POI, in a host cell, also referred to as production cell, wherein the method comprises controlling the level of Pat1 in the cell. Specifically, the level is reduced by a genetic modification which comprises eliminating, e.g., knocking out, a copy of the gene encoding the Pat1. In some embodiments, reducing the level of Pat1 in the cell, e.g., production cell, comprises eliminating, e.g., knocking out, all (e.g., both) copies of the gene encoding Pat1, in particular the endogenous Pat1, from the genome of the cell. In some embodiments, reducing the level of Pat1, in particular, the endogenous Pat1 in the cell, e.g., production cell, comprises eliminating, e.g., knocking out, a regulatory nucleic acid sequence, e.g., promoter or enhancer, operably coupled to the gene encoding Pat1. In some embodiments, reducing the level of Pat1 in the cell, e.g., production cell level, comprises eliminating, e.g., knocking out, all (e.g., both) copies of a regulatory nucleic acid sequence, e.g., promoter or enhancer, operably coupled to the gene encoding Pat1. In some embodiments, eliminating, e.g., knocking out, comprises introducing a deletion, substitution, or insertion mutation to the gene encoding Pat1 or to the regulatory nucleic acid sequence, e.g., promoter or enhancer, operably coupled to the gene encoding Pat1, e.g., wherein the mutation reduces the level of expression of Pat1. In some embodiments, eliminating, e.g., knocking out, comprises introducing a deletion or insertion of 1, 2, 3, 4, 5, 6, 7, 8 9, 10, or more base pairs in the gene encoding Pat1 or in the regulatory nucleic acid sequence, e.g., promoter or enhancer, operably coupled to the gene encoding Pat1. In some embodiments, eliminating, e.g., knocking out, comprises substituting 1, 2, 3, 4, 5, 6, 7, 89, 10, or more base pairs for different base pairs (e.g., transition or transversion mutations) in the gene encoding Pat1 or the regulatory nucleic acid sequence, e.g., promoter or enhancer, operably coupled to the gene encoding Pat1. For example, an insertion mutation that disrupts the reading frame of the gene encoding Pat1 or a substitution mutation that alters the start codon of the gene encoding Pat1 would both be examples of eliminating, e.g., knocking out, that reduce the level of Pat1. As a further example, eliminating, e.g., knocking out, may comprise insertion of a marker (e.g., an antibiotic resistance, auxotrophy mitigating, or fluorescent marker encoding gene) in a manner that deletes a promoter or portion of a promoter operably linked to the gene encoding Pat1. In some embodiments, eliminating, e.g., knocking out, results in the complete loss of production of Pat1 from the copy of the gene encoding the Pat1. In other embodiments, eliminating, e.g., knocking out, results in a lower level of production of the Pat1, e.g., of functional Pat1. In other embodiments, eliminating, e.g., knocking out, results in production of a variant of Pat1 that is one or more of: truncated, less-functional, non-functional, misfolded, and/or degradation-prone. Eliminating, e.g., knocking out, a copy of a gene encoding Pat1 or a regulatory element operably linked to said gene may be achieved by means of any gene editing system known in the art. Exemplary gene editing systems include clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol.31.7(2013):397-405; CRISPR methods of gene editing are described, e.g., in Guan et al., Application of CRISPR-Cas system in gene therapy: Pre-clinical progress in animal model. DNA Repair 2016 July 30 [Epub]; Zheng et al., Precise gene deletion and replacement using the CRISPR/Cas9 system in human cells. BioTechniques, Vol. 57, No. 3, September 2014, pp. 115-124. A person of skill in the art will be aware of these and other options for editing the genome (e.g., knocking out a gene) in a cell, e.g., a production cell. In some embodiments, eliminating, e.g., knocking out, a copy of a gene encoding Pat1 or a regulatory element operably linked to said gene comprises using a CRISPR-Cas9 molecule, e.g., a Cas9 molecule, in combination with an RNA (e.g., gRNA, sgRNA, and/or tracrRNA), specific for the Pat1 encoding gene or a regulatory nucleic acid sequence, e.g., promoter or enhancer, operably coupled to the gene encoding Pat1. In some embodiments, reducing the level of an endogenous protein in the cell, e.g., production cell, comprises reducing the level of mRNA transcript encoding the endogenous protein, e.g., knockdown, in the cell. In some embodiments, reducing the level of mRNA transcript encoding the endogenous protein, e.g., knockdown, comprises using a siRNA, e.g., capable of binding to nucleic acid, e.g., mRNA, encoding the endogenous protein or a regulatory nucleic acid sequence, e.g., promoter or enhancer, operably linked thereto. A person of skill in the art will be aware of tools and techniques for designing and delivering siRNA to a cell. Such tools and/or techniques include, but are not limited to: Dharmacon Horizon siDesign tool, InvivoGen siRNA Wizard, GenScript siRNA Construct Services, IDT Custom Dicer-Substrate siRNA, Sigma-Aldrich siRNA Design Service. In some embodiments, reducing the level of mRNA transcript encoding Pat1, e.g., knockdown, results in a reduction of at least 10, 20, 30, 40, 50, 60, 70, 80, 90, or 100% in the level of mRNA transcript encoding Pat1 in the cell.

A complete inhibition of expression of a given gene is referred to as “knockout”. Knockout of a gene means that no functional transcripts are synthesized from said gene leading to a loss of function normally provided by this gene. Gene knockout is achieved by altering the DNA sequence leading to disruption or deletion of the gene or its regulatory sequences, or part of such gene or regulatory sequences. Knockout technologies include the use of homologous recombination techniques to replace, interrupt or delete crucial parts or the entire gene sequence or the use of DNA-modifying enzymes such as zinc-finger or mega-nucleases to introduce double strand breaks into DNA of the target gene e.g., described by Gaj et al. (Trends Biotechnol. 2013; 31(7):397-405).

Specific embodiments employ one or more knockout plasmids or cassettes which are transformed or transfected into the host cells. By homologous recombination the target gene in the host cells can be disrupted. This procedure is typically repeated until all alleles of the target gene are stably removed.

Knocking out a copy of a gene encoding an endogenous protein (such as the Pat1) or a regulatory element operably linked to said gene may be achieved by means of any gene editing system known in the art. Exemplary gene editing systems include clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods.

One specific method for knocking out a specific gene as described herein is the CRISPR-Cas9 methods as described in e.g., Weninger et al. (J. Biotechnol. 2016, 235:139-49). Another method includes the split marker approach as described by e.g. Heiss et al. 2013 (Appl Microbiol Biotechnol. 97(3):1241-9.)

According to a specific example, the knocking out is performed by using a CRISPR-Cas9 molecule, e.g., a Cas9 molecule in combination with a RNAg, gRNA, sgRNA, and/or tracrRNA specific for an endogenous protein (such as the Pat1 protein) encoding gene or a regulatory nucleic acid sequence, e.g., promoter or enhancer operably coupled to the gene coding endogenous protein. The level of an endogenous protein (such as the Pat1 protein) in a cell can be reduced by using a CRISPR-Cas9 molecule such as Cas9 molecule and RNA, such as gRNA, sgRNA, and/or tracrRNA that is specific for endogenous protein encoding gene or regulatory nucleic acid sequence, e.g., promoter or enhancer operably coupled to the gene coding endogenous protein, or by providing a nucleic acid encoding the CRISPR-Cas9 molecule and RNA that is specific for the endogenous protein encoding gene or regulatory nucleic acid sequence, e.g., promoter or enhancer operably coupled to the gene encoding the endogenous protein.

Another embodiment refers to target mRNA degradation by using small interfering RNA (siRNA) to transfect the host cell and targeting a mRNA encoding the target protein expressed endogenously by said host cell. The level of an endogenous protein (such as the Pat1 protein) in a cell can be reduced by using a siRNA capable of binding to the nucleic acid encoding the endogenous protein or a regulatory nucleic acid sequence, e.g., promoter or enhancer operably linked to it. The siRNA can be used by providing siRNA in a cell, or providing a nucleic acid encoding the siRNA in the cell. Specifically, the cell comprises a siRNA capable of binding to a nucleic acid encoding the endogenous protein or a respective regulatory element. The siRNA can be designed, selected, produced by a method selected from or equivalent to Dharmacon Horizon siDesign tool, invivoGen siRNA Wizard, GenScript siRNA Construct Services, IDT Custom Dicer-Substrate siRNA, Sigma-Aldrich siRNA Design Service, or commercially available sources.

Expression of a gene may be inhibited or reduced by methods which directly interfere with gene expression, encompassing, but not restricted to, inhibition or reduction of DNA transcription, e.g., by use of specific promoter-related repressors, by site specific mutagenesis of a given promoter, by promoter exchange, or inhibition or reduction of translation, e.g., by RNAi or non-coding RNA induced post-transcriptional gene silencing. The expression of a dysfunctional, or inactive gene product with reduced activity, can, for example, be achieved by site specific or random mutagenesis, insertions or deletions within the coding gene.

The inhibition or reduction of the activity of gene product can, for example, be achieved by administration of, or incubation with, an inhibitor to the respective enzyme, prior to or simultaneously with protein expression. Examples for such inhibitors include, but are not limited to, an inhibitory peptide, an antibody, an aptamer, a fusion protein or an antibody mimetic against said enzyme, or a ligand or receptor thereof, or an inhibitory peptide or nucleic acid, or a small molecule with similar binding activity.

Gene silencing, gene knock-down and gene knockout refers to techniques by which the expression of a gene is reduced, either through genetic modification or by treatment with an oligonucleotide with a sequence complementary to either an mRNA transcript or a gene. If genetic modification of DNA is done, the result is a knock-down or knockout organism. If the change in gene expression is caused by an oligonucleotide binding to an mRNA or temporarily binding to a gene, this results in a temporary change in gene expression without modification of the chromosomal DNA and is referred to as a transient knock-down.

In a transient knock-down, which is also encompassed by the above term, the binding of this oligonucleotide to the active gene or its transcripts causes decreased expression through blocking of transcription (in the case of gene-binding), degradation of the mRNA transcript (e.g., by small interfering RNA (siRNA) or antisense RNA) or blocking mRNA translation.

Other approaches to carry out gene silencing, knock-down or knockout are known to the skilled person from the respective literature, and their application in the context of the present invention is considered as routine. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), or Vega et al., Gene Targeting, CRC Press, Ann Arbor Mich. (1995).

Gene knockout refers to techniques by which the expression of a gene is fully blocked i.e., the respective gene is inoperative, or even removed. Methodological approaches to achieve this goal are manifold and known to the skilled person. Examples are the production of a mutant which is dominantly negative for the given gene. Such mutant can be produced by site directed mutagenesis (e.g., deletion, partial deletion, insertion or nucleic acid substitution), by use of suitable transposons, or by other approaches which are known to the skilled person from the respective literature, the application of which in the context of the present invention is thus considered as routine. One example is knockout by use of targeted Zinc Finger Nucleases. A respective Kit is provided by Sigma Aldrich as “CompoZR knockout ZFN”. Another approach encompasses the use of Transcription activator-like effector nucleases (TALENs).

The delivery of a dominant negative construct involves the introduction of a sequence coding for a dysfunctional gene expression product e.g., by transfection. Said coding sequence is functionally coupled to a strong promoter, in such way that the gene expression of the dysfunctional enzyme overrules the natural expression of the gene expression product, which, in turn, leads to an effective physiological defect of the respective activity of said gene expression product.

A conditional gene knockout allows blocking gene expression in a tissue- or time-specific manner. This is done, for example, by introducing short sequences called IoxP sites around the gene of interest. Again, other approaches are known to the skilled person from the respective literature, and their application in the context of the present invention is considered as routine.

One other approach is gene alteration which may lead to a dysfunctional gene product or to a gene product with reduced activity. This approach involves the introduction of frame shift mutations, nonsense mutations (i.e., introduction of a premature stop codon) or mutations which lead to an amino acid substitution which renders the whole gene product dysfunctional, or causing a reduced activity. Such gene alteration can for example be produced by mutagenesis (e.g., deletion, partial deletion, insertion or nucleic acid substitution), either unspecific (random) mutagenesis or site directed mutagenesis. Protocols describing the practical application of gene silencing, gene knock-down, gene knockout, delivery of a dominant negative construct, conditional gene knockout, and/or gene alteration are commonly available to the skilled artisan, and are within his routine. The technical teaching provided herein is thus entirely enabled with respect to all conceivable methods leading to an inhibition or reduction of gene expression of a gene product, or to the expression of a dysfunctional, or inactive gene product, or with reduced activity.

Genetic modifications described herein may employ tools, methods and techniques known in the art, such as described by Sambrook et al., Molecular Cloning: A Laboratory Manual, Cold Springs Harbor Laboratory, New York (1989, 1992), Ausubel et al., Current Protocols in Molecular Biology, John Wiley and Sons, Baltimore, Md. (1989), or Vega et al., Gene Targeting, CRC Press, Ann Arbor Mich. (1995).

The term “endogenous” as used herein is meant to include those molecules and sequences, in particular endogenous genes or proteins, which are present in the wild-type (native) host cell, prior to its modification to reduce expression of the respective endogenous genes and/or reduce the production of the endogenous proteins. In particular, an endogenous nucleic acid molecule (e.g., a gene) or protein that does occur in (and can be obtained from) a particular host cell as it is found in nature, is understood to be “host cell endogenous” or “endogenous to the host cell”. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature.

Thus, even if an endogenous protein is no more produced by a host cell, such as in a knockout mutant of the host cell, where the protein encoding gene is inactivated or deleted, the protein is herein still referred to as “endogenous”.

The term “heterologous” as used herein with respect to a nucleotide sequence, construct such as an expression cassette, amino acid sequence or protein, refers to a compound which is either foreign to a given host cell, i.e. “exogenous”, such as not found in nature in said host cell; or that is naturally found in a given host cell e.g., is “endogenous”, however, in the context of a heterologous construct or integrated in such heterologous construct e.g., employing a heterologous nucleic acid fused or in conjunction with an endogenous nucleic acid, thereby rendering the construct heterologous. The heterologous nucleotide sequence as found endogenously may also be produced in an unnatural e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a nucleotide sequence not natively associated with a promoter e.g., to obtain a hybrid promoter, or operably linked to a coding sequence, as described herein. As a result, a hybrid or chimeric polynucleotide may be obtained. A further example of a heterologous compound is a POI encoding polynucleotide operably linked to a transcriptional control element e.g., a promoter, to which an endogenous, naturally-occurring POI coding sequence is not normally operably linked.

The term “mutagenesis” as used herein shall refer to a method of providing mutants of a nucleotide sequence e.g., through insertion, deletion and/or substitution of one or more nucleotides, so to obtain variants thereof with at least one change in the non-coding or coding region. Mutagenesis may be through random, semi-random or site directed mutation. Specific ECP described herein and respective nucleotide sequences may be used to produce variants, which are likewise regulatable promoters which may be used for the purpose as described herein. Such variants can be produced by a suitable mutagenesis method using the ECP nucleotide sequences provided herein as a parent sequence. Such mutagenesis method encompasses those methods of engineering the nucleic acid or de novo synthesizing a nucleotide sequence using the respective parent promoter sequence information as a template. Specific mutagenesis methods apply rational promoter engineering.

The exemplary ECP described herein may e.g., be modified to generate promoter variants with altered expression levels and regulatory properties. For instance, a promoter library may be prepared by mutagenesis of selected promoter sequences, which may be used as parent molecules e.g., to fine-tune the gene expression in eukaryotic cells by analyzing variants for their expression under different fermentation strategies and selecting suitable variants. A synthetic library of variants may be used e.g., to select a promoter matching the requirements for producing a selected POI. Such variants may have increased expression efficiency in (e.g., eukaryotic) host cells and differential expression under carbon source rich and limiting conditions. Typically, large randomized gene libraries are produced with a high gene diversity, which may be selected according to a specifically desired genotype or phenotype.

Certain ECP variants may be size variants of the ECP nucleotide sequences provided herein and/or comprise more than one of the elements or regions of the promoter described herein, such as the core regulatory regions, the main regulatory regions, or the T motifs, and/or comprise one or more (of the same or different) fragments of the ECP nucleotide sequences.

Specific mutagenesis methods provide for point mutations of one or more nucleotides in a sequence, in particular tandem point mutations, such as to change at least 2, 3, 4, 5, 6, 7, 8, 9, 10, or even more continuous nucleotides within the nucleotide sequence of the promoter. A point mutation is typically at least one of a deletion, insertion, and/or substitution of one or more nucleotides. The promoter sequence may be mutated at the distal ends, in particular within the 5′-region which amounts to up to 50% of the full-length promoter sequence, which 5′-region can be highly variable without substantially losing the promoter activity. The promoter sequence may specifically be mutated within the main regulatory region, yet, it may be preferred that the sequence identity to the exemplary main regulatory region and in particular to the exemplary core regulatory region is high, such as e.g., at least any one of 80%, 85%, 90%, or 95%. Outside any of the core or main regulatory regions, the variability of the sequence may be higher and the ECP still be functional e.g., with a sequence identity of less than 80% or less than 85%.

Any mutation within the core or main regulatory regions is typically conservative, such as to maintain (or even improve) the recognition by a certain transcription factor.

Specifically, the ECP described herein may comprise a hybrid nucleotide sequence e.g. comprising the core or main regulatory regions described herein and in addition one or more regions or alternative (native or artificial) promoter sequences, such as an translation initiation site at the 3′-region (specifically the 3′-end which comprises at least 10 or 15 3′-terminal nucleotide sequence including the 3′-terminus, (e.g., up to 20, 25, or 30 nt) of a different promoter, e.g. of any constitutive or regulatable (or otherwise inducible) promoter, thereby substituting the translation initiation site of the ECP promoter.

The term “operably linked” as used herein refers to the association of nucleotide sequences on a single nucleic acid molecule, e.g., a vector, or an expression cassette, in a way such that the function of one or more nucleotide sequences is affected by at least one other nucleotide sequence present on said nucleic acid molecule. By operably linking, a nucleic acid sequence is placed into a functional relationship with another nucleic acid sequence on the same nucleic acid molecule. For example, a promoter is operably linked with a coding sequence of a recombinant gene, when it is capable of effecting the expression of that coding sequence. As a further example, a nucleic acid encoding a signal peptide is operably linked to a nucleic acid sequence encoding a POI, when it is capable of expressing a protein in the secreted form, such as a preform of a mature protein or the mature protein. Specifically, such nucleic acids operably linked to each other may be immediately linked i.e., without further elements or nucleic acid sequences in between the nucleic acid encoding the signal peptide and the nucleic acid sequence encoding a POI. Alternatively, a suitable linking sequence can be used such as e.g., a cloning site positioned between the promoter and the GOI.

The term “polynucleotide”, “nucleic acid molecule(s)” or “nucleic acid sequence(s)” as interchangeably used herein, refers to nucleotides, either ribonucleotides or deoxyribonucleotides or a combination of both, in a polymeric unbranched form of any length. Preferably, a polynucleotide refers to deoxyribonucleotides in a polymeric unbranched form of any length. Here, nucleotides consist of a pentose sugar (deoxyribose), a nitrogenous base (adenine, guanine, cytosine or thymine) and a phosphate group.

A “promoter” sequence is typically understood as a non-coding regulatory sequence which, when operably linked to a coding sequence, controls the transcription of the coding sequence. A promoter sequence may be natively associated with the coding sequence, such as in a native (wild-type) cell for endogenous protein expression.

A promoter is herein described to initiate, regulate, or otherwise mediate or control the expression of a protein coding polynucleotide (DNA), such as a POI coding DNA. Promoter DNA and coding DNA may be or be derived from the same gene or from different genes, and may be derived from the same or different organisms.

Either the promoter or the coding sequence, or both, can be heterologous to the cell. A promoter may or may not be natively associated with the coding sequence. Any one or both of the promoter and the coding sequence can be endogenous and are herein also understood to be not natively associated in a cell, if comprised in a heterologous expression cassette.

A heterologous promoter may be heterologous to the polynucleotide to be expressed and/or an artificial promoter, or a promoter that is originating from the wild-type host cell, but positioned in the host cell genome within a heterologous expression cassette or positioned at a location where it is not naturally-occurring in the wild-type host cell.

The strength of a promoter specifically refers to its transcription strength, represented by the efficiency of initiation of transcription occurring at that promoter with high or low frequency. The higher the transcription strength, the more frequently transcription will occur at that promoter. Promoter strength is a typical feature of a promoter, because it determines how often a given mRNA sequence is transcribed, effectively giving higher priority for transcription to some genes over others, leading to a higher concentration of the transcript. A gene that codes for a protein that is required in large quantities, for example, typically requires a relatively strong promoter. The RNA polymerase can only perform one transcription task at a time and so must prioritize its work to be efficient. Differences in promoter strength are selected to allow for this prioritization.

The promoter strength may also refer to the frequency of transcription which is commonly understood as the transcription rate e.g., as determined by the amount of a transcript in a suitable assay e.g., RT-PCR or Northern blotting. For example, the transcription strength of a promoter described herein is determined in the host cell which is P. pastoris and compared to the native pGAP promoter of P. pastoris.

The native pGAP promoter typically initiates expression of the gap gene encoding glyceraldehyde-3-phosphate dehydrogenase (GAPDH), which is a constitutive promoter present in most living organisms. GAPDH (EC 1.2.1.12), a key enzyme of glycolysis and gluconeogenesis, plays a crucial role in catabolic and anabolic carbohydrate metabolism.

The term “regulatable” with respect to an inducible or repressible regulatory element, such as a promoter described herein shall refer to an element that is repressed in a host cell in the presence of an excess amount of a substance (such as a nutrient in the cell culture medium) e.g., in the growth phase of a batch culture, and de-repressed to induce strong activity e.g., in the production phase (such as upon reducing the amount of a nutrient, or upon feeding of a supplemental substrate), according to a fed-batch strategy. A regulatory element can as well be designed to be regulatable, such that the element is inactive without addition of a cell culture additive, and active in the presence of such additive. Thus, expression of a POI under the control of such regulatory element can be induced upon addition of such additive.

The term “repression,” or “repressed,” as used herein e.g., to characterize a carbon-source regulatable promoter described herein, refers to the interference of transcription of a gene of interest (encoding a protein of interest) that is under the transcriptional control of a promoter that is understood to be repressible, resulting in decreased expression of the protein of interest by the cell(s).

Repression of an ECP as described herein is specifically occurring when a repressing agent is in the cell culture medium. A repressing agent can be a certain carbon-source or a repressing amount of a carbon-source e.g., above a certain threshold amount. Expression of a gene of interest or of a protein of interest is said to be “derepressed,” when, the repressing agent is removed from the medium, or reduced to below a threshold amount that is no more repressing. Upon derepressing, the ECP is understood to be fully induced, and expression of the protein of interest is typically at least 1.5-fold over the basal levels of expression by the cell(s) under promoter-repressing conditions.

Specifically, transcription of a gene of interest under the control of a carbon-source regulatable ECP described herein may be repressed by at least any one of 30, 40, 50, 60, 70, 80, 85%, 90%, or 95%, or completely repressed (100% repressed) compared to transcription of said gene upon de-repressing or fully inducing the ECP.

The differential promoter strength comparing the promoter strength under repressed and derepressed condition, determines the regulatable properties of a promoter and the respective induction ratio. According to certain embodiments, the induction ratio is understood as a differential promoter strength which is determined by the initiation of POI production upon switching to inducing conditions below a predetermined carbon source threshold, and compared to the strength in the repressed state. The transcription strength commonly is understood as the strength in the fully induced state i.e., showing about maximum activities under de-repressing conditions. The differential promoter strength is e.g., determined according to the efficiency or yield and/or titer of POI production in a recombinant host cell line under de-repressing conditions as compared to repressing conditions, or else by the amount of a transcript. The regulatable promoter as described herein has a preferred differential promoter strength (induction ratio), which is at least 1.5-fold or at least 2-fold, more preferably at least 5-fold, even more preferred at least 10-fold, more preferred at least 20-fold, more preferably at least 30, 40, 50, or 100-fold in the de-repressed (fully induced) state compared to the repressed state, also understood as fold induction.

The term “Pat1” as used herein shall refer to an evolutionarily conserved multi-domain RNA binding protein and eukaryotic P-body component, known to interact with mRNA decapping and decay factors to coordinate translation repression, P-body formation and mRNA decay.

Pat1 (short for protein associated with topoisomerase II, name given by Wang et al. 1996; Nucleic Acids Research 24(23):4791-4797) is described as deadenylation-dependent mRNA-decapping factor, involved in P-body (cytoplasmic mRNA processing body) formation. Wang et al describe Pat1 of S. cerevisiae. The human homolog is called PatL1 or Pat1b.

Alternative names for Pat1 are:
Decapping activator and translational repressor PAT1
Topoisomerase II-associated protein PAT1
mRNA turnover protein 1

Marnef et al. (Biochem Soc Trans. 2010; 38(6):1602-7) also describes Pat1 functions: Pat1 proteins: a life in translation, translation repression and mRNA decay. Pat1 proteins are conserved across eukaryotes. Vertebrates have evolved two Pat1 proteins paralogues, whereas invertebrates and yeast only possess one such protein. Despite their lack of known domains or motifs, Pat1 proteins are involved in several key post-transcriptional mechanisms of gene expression control. In yeast, Pat1p interacts with translating mRNPs (messenger ribonucleoproteins), and is responsible for translational repression and decapping activation, ultimately leading to mRNP degradation. Drosophila HPat and human Pat1b (PatL1) proteins also have conserved roles in the 5′→3′ mRNA decay pathway. Consistent with their functions in silencing gene expression, Pat1 proteins localize to P-bodies (processing bodies) in yeast, Drosophila, Caenorhabditis elegans and human cells.

mRNA decapping requires the functions of specific regulators commonly known as “decapping activators”, such as Pat1, Dhh1 and the Lsm1-7 complex (Parker, Genetics 2012, 191:671-702). The two major proposed functions for decapping activators are translational repression and decapping enzyme activation.

Specifically, the Pat1 protein is a DNA topoisomerase 2-associated protein.

Specifically, the Pat1 protein is a topoisomerase II-associated deadenylation-dependent mRNA-decapping factor.

Pat1 is described to regulate general translational repression. It is described as a translational activator of select mRNAs during filamentous growth, mating and autophagy. It is described to cooperate with Ngr1p to promote specific mRNA decay. ATP- and RNA-bound form promote processing body assembly, while ATPase stimulation by Not1p promotes disassembly; forms cytoplasmic foci on replication stress. Exemplary Pat1 proteins are listed in the table 2 below, showing sequence identity to Pat1 of K. phaffii. Mammalian Pat1 orthologs are also referred to as PAT1-like 1, or PATL1, or Pat1.

List of exemplary Pat1 proteins:

| | | NCBI |
|---|---|---|
| | | Database |
| Gene name | Organism | Accession no.* |
| topoisomerase II associated protein (Pat1) | Aspergillus niger CBS 513.88 | XP_001401013.2 |
| [Aspergillus niger CBS 513.88] | | |
| protein PAT1 homolog 1 isoform X4 | Cricetulus griseus | XP_035297861.1 |
| [Cricetulus griseus] | | |
| protein PAT1 homolog 1 isoform X5 | Cricetulus griseus | XP_027262440.1 |
| [Cricetulus griseus] | | |
| protein PAT1 homolog 1 isoform X3 | Cricetulus griseus | XP_027262439.1 |
| [Cricetulus griseus] | | |
| protein PAT1 homolog 1 isoform X2 | Cricetulus griseus | XP_035297860.1 |
| [Cricetulus griseus] | | |
| Protein PAT1-like 1, Patl1 [Cricetulus griseus] | Cricetulus griseus | EGW13051.1 |
| Protein associated with topoisomerase = | Homo sapiens | AAH65264.2 |
| homolog 1 (yeast) [Homo sapiens] | | |
| protein PAT1 homolog 1 [Homo sapiens] | Homo sapiens | NP_689929. |
| Pat1 [Kluyveromyces lactis] | Kluyveromyces lactis | QEU58038.1 |
| BA75_02274T0 [Komagataella pastoris] | Komagataella pastoris | ANZ75923.1 |
| Deadenylation-dependent mRNA-decapping | Komagataella phaffii CBS | CCA38092.1 |
| factor [Komagataella phaffii CBS 7435] | 7435 | |
| uncharacterized protein | Ogataea polymorpha | XP_018211124.1 |
| OGAPODRAFT_75821 [Ogataea polymorpha] | | |
| Pat1p [Saccharomyces cerevisiae S288C] | Saccharomyces cerevisiae | NP_010002.3 |
| | S288C | |
| putative topoisomerase II-associated | Schizosaccharomyces | NP_595976.2 |
| deadenylation-dependent mRNA-decapping | pombe | |
| factor [Schizosaccharomyces pombe] | | |
| hypothetical protein M419DRAFT_108256 | Trichoderma reesei | ETS04809.1 |
| [Trichoderma reesei RUT C-30] | RUT C-30 | |
| DNA topoisomerase 2-associated protein | Yarrowia lipolytica | QNP99077.1 |
| pat1 [Yarrowia lipolytica] | | |

*National Library of Medicine, Bethesda, MD

Sequence identity of the exemplary Pat1 proteins
compared to Pat1 of K. phaffii (SEQ ID NO: 1):

| | | Max | Total | Query | E | Percent | Acc. |
|---|---|---|---|---|---|---|---|
| Organism | Accession | Score | Score | Cover | value | Ident | Len |
| Aspergillus niger CBS | XP_001401013.2 | 162 | 162 | 54% | 8E−40 | 27.1% | 837 |
| 513.88 | | | | | | | |
| Cricetulus griseus | XP_035297861.1 | 43.9 | 43.9 | 29% | 0.009 | 23.4% | 686 |
| Cricetulus griseus | XP_027262440.1 | 43.9 | 43.9 | 29% | 0.01 | 23.4% | 685 |
| Cricetulus griseus | XP_027262439.1 | 43.1 | 43.1 | 29% | 0.019 | 23.4% | 770 |
| Cricetulus griseus | XP_035297860.1 | 43.1 | 43.1 | 29% | 0.019 | 23.4% | 771 |
| Cricetulus griseus | EGW13051.1 | 43.9 | 43.9 | 29% | 0.012 | 23.4% | 695 |
| Homo sapiens | AAH65264.2 | 43.1 | 43.1 | 33% | 0.018 | 22.3% | 627 |
| Homo sapiens | NP_689929. | 42.4 | 42.4 | 33% | 0.036 | 22.2% | 770 |
| Kluyveromyces lactis | QEU58038.1 | 347 | 347 | 64% | 8E−105 | 41.6% | 870 |
| Komagataella pastoris | ANZ75923.1 | 1477 | 1477 | 99% | 0 | 92.1% | 845 |
| Komagataella phaffii | CCA38092.1 | 1573 | 1573 | 99% | 0 | 100.0% | 833 |
| CBS 7435 | | | | | | | |
| Ogataea polymorpha | XP_018211124.1 | 407 | 407 | 59% | 2E−128 | 49.2% | 797 |
| Saccharomyces | NP_010002.3 | 320 | 366 | 64% | 2E−95 | 39.6% | 796 |
| cerevisiae S288C | | | | | | | |
| Schizosaccharomyces | NP_595976.2 | 148 | 148 | 53% | 2E−35 | 26.5% | 754 |
| pombe | | | | | | | |
| Trichoderma reesei | ETS04809.1 | 155 | 155 | 57% | 2E−37 | 27.9% | 843 |
| RUT C-30 | | | | | | | |
| Yarrowia lipolytica | QNP99077.1 | 228 | 228 | 58% | 1E−61 | 34.2% | 850 |

The term “protein of interest (POI)” as used herein refers to a polypeptide or a protein that is produced by means of recombinant technology in a host cell. More specifically, the protein may either be a polypeptide not naturally-occurring in the host cell i.e., a heterologous protein, or else may be native to the host cell i.e., a homologous protein to the host cell, but is produced, for example, by transformation or transfection with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g., of the promoter sequence. In some cases, the term POI as used herein also refers to any metabolite product by the host cell as mediated by the recombinantly expressed protein.

As referred to herein, a POI may be specifically selected from the following: BOTOX, Myobloc, Neurobloc, Dysport (or other serotypes of botulinum neurotoxins), alglucosidase alpha, daptomycin, YH-16, choriogonadotropin alpha, filgrastim, cetrorelix, interleukin-2, aldesleukin, teceleulin, denileukin diftitox, interferon alpha-n3 (injection), interferon alpha-nl, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alpha, collagenase, carperitide, recombinant human epidermal growth factor (topical gel, wound healing), DWP401, darbepoetin alpha, epoetin omega, epoetin beta, epoetin alpha, desirudin, lepirudin, bivalirudin, nonacog alpha, Mononine, eptacog alpha (activated), recombinant Factor VIII+VWF, Recombinate, recombinant Factor VIII, Factor VIII (recombinant), Alphnmate, octocog alpha, Factor VIII, palifermin, indikinase, tenecteplase, alteplase, pamiteplase, reteplase, nateplase, monteplase, follitropin alpha, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, Eutropin, KP-102 program, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alpha, trafermin, ziconotide, taltirelin, diboterminalfa, atosiban, becaplermin, eptifibatide, Zemaira, CTC-111, Shanvac-B, HPV vaccine (quadrivalent), octreotide, lanreotide, ancestirn, agalsidase beta, agalsidase alpha, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin Ill, Granditropin, Vitrase, recombinant insulin, interferon-alpha (oral lozenge), GEM-21S, vapreotide, idursulfase, omnapatrilat, recombinant serum albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alpha-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 1311-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, ACM-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10 (autoimmune diseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alpha-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague FIV vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, HA, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhlGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36](obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-1, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S. pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, or TP-9201, adalimumab (HUMIRA), infliximab (REMICADE™), rituximab (RITUXAN™/MAB THERA™), etanercept (ENBREL™), bevacizumab (AVASTIN™), trastuzumab (HERCEPTIN™), pegrilgrastim (NEULASTA™), or any other suitable POI including biosimilars and biobetters.

The term “sequence identity” of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or genes by detecting excess similarity, and statistically significant similarity that reflects common ancestry. Homologues may encompass orthologues, which are herein understood as the same protein in different organisms, e.g., variants of such protein in different different organisms or species.

“Percent (%) amino acid sequence identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences is determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver (https://www.ebi.ac.uk/Tools/psa/emboss_needle/) was used with default settings (Matrix: EBLOSUM62; Gap open:10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

“Percent (%) identity” with respect to a nucleotide sequence e.g., of a promoter or a gene, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences is determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2,-3, Filter string: Low complexity regions, Mark for lookup table only.

The term “isolated” or “isolation” as used herein with respect to a POI shall refer to such compound that has been sufficiently separated from the environment with which it would naturally be associated, in particular a cell culture supernatant, so as to exist in “purified” or “substantially pure” form. Yet, “isolated” does not necessarily mean the exclusion of artificial or synthetic mixtures with other compounds or materials, or the presence of impurities that do not interfere with the fundamental activity, and that may be present, for example, due to incomplete purification. Isolated compounds can be further formulated to produce preparations thereof, and still for practical purposes be isolated—for example, a POI can be mixed with pharmaceutically acceptable carriers or excipients when used in diagnosis or therapy.

The term “purified” as used herein shall refer to a preparation comprising at least 50% (mol/mol), preferably at least 60%, 70%, 80%, 90% or 95% of a compound (e.g., a POI). Purity is measured by methods appropriate for the compound (e.g., chromatographic methods, polyacrylamide gel electrophoresis, HPLC analysis, and the like). An isolated, purified POI as described herein may be obtained by purifying the cell culture supernatants to reduce impurities.

As isolation and purification methods for obtaining a recombinant polypeptide or protein product, methods, such as methods utilizing difference in solubility, such as salting out and solvent precipitation, methods utilizing difference in molecular weight, such as ultrafiltration and gel electrophoresis, methods utilizing difference in electric charge, such as ion-exchange chromatography, methods utilizing specific affinity, such as affinity chromatography, methods utilizing difference in hydrophobicity, such as reverse phase high performance liquid chromatography, and methods utilizing difference in isoelectric point, such as isoelectric focusing may be used.

The following standard methods are preferred: cell (debris) separation and wash by Microfiltration or Tangential Flow Filter (TFF) or centrifugation, POI purification by precipitation or heat treatment, POI activation by enzymatic digest, POI purification by chromatography, such as ion exchange (IEX), hydrophobic interaction chromatography (HIC), affinity chromatography, size exclusion (SEC) or HPLC chromatography, POI precipitation, concentration and washing, such as by ultrafiltration steps.

A highly purified product is essentially free from contaminating proteins, and preferably has a purity of at least 90%, more preferred at least 95%, or even at least 98%, up to 100%. The purified products may be obtained by purification of the cell culture supernatant or else from cellular debris.

An isolated and purified POI can be identified by conventional methods such as Western blot, HPLC, activity assay, or ELISA.

The term “recombinant” as used herein shall mean “being prepared by or the result of genetic engineering. A “recombinant cell” or “recombinant host cell” is herein understood as a cell or host cell that has been genetically engineered or modified to comprise a nucleic acid sequence which was not native to said cell. A recombinant host may be engineered to delete and/or inactivate one or more nucleotides or nucleotide sequences, and may specifically comprise an expression vector or cloning vector containing a recombinant nucleic acid sequence, in particular employing nucleotide sequence foreign to the host. A recombinant protein is produced by expressing a respective recombinant nucleic acid in a host. The term “recombinant” with respect to a POI as used herein, includes a POI that is prepared, expressed, created or isolated by recombinant means, such as a POI isolated from a host cell transformed or transfected to express the POI. In accordance with the present invention conventional molecular biology, microbiology, and recombinant DNA techniques within the skill of the art may be employed. Such techniques are explained fully in the literature. See, e.g., Sambrook et al., 2012, Molecular Cloning: A Laboratory Manual, volumes 1-4, Cold Spring Harbor Press, NY).

Certain recombinant host cells are “engineered” host cells which are understood as host cells which have been manipulated using genetic engineering i.e., by human intervention. When a host cell is engineered to express, underexpress or knockout a given gene or the respective protein, the host cell is manipulated such that the host cell has the capability to express such gene and protein, respectively, to a different extent compared to the host cell under the same condition prior to manipulation, or compared to the host cells which are not engineered. Cells that are not engineered by any recombinant means or techniques are generally understood as being naturally-occurring or wild-type.

Therefore, the present invention provides for an improved POI production by controlling expression of an mRNA decapping activator in the host cell.

According to specific examples, it has been found that deletion of pat1 significantly affects eukaryotic host cells, in particular P. pastoris. The engineered strains exhibit increased global translation activity in addition to strongly increased recombinant protein production. Thus, a new cell engineering strategy has been developed which allows for much higher product formation.

To decrease global protein synthesis repression upon glucose limitation, the gene coding for Pat1 (herein referred to as pat1 or PAT1) was deleted in various strains. By pat1 deletion, production of model proteins (such as an antibody fragment or human serum albumin) was increased at least 3-fold (or even 6-fold) depending on the protein and strain. Measurements of global translation activity showed correspondingly increased levels in the engineered cells versus the comparison strains (without such engineering for pat1 deletion. Therefore, the engineering strategy to control Pat1 expression indeed increases mRNA translation cell-wide.

It can be concluded that expression control of a decapping activator such as Pat1 leads to a global higher translational activity of a eukaryotic host cell such as P. pastoris, enabling increased productivity of recombinant proteins.

The present invention is specifically described by one or more of the following items:
1. A eukaryotic host cell which is engineered to introduce a recombinant expression cassette expressing a gene of interest (GOI) to produce a protein of interest (POI), and by a genetic modification to control expression of a Pat1 protein compared to an endogenous expression thereof.
2. The host cell of item 1, wherein the expression of the Pat1 protein is reduced by one or more genetic modifications comprising a disruption, substitution, deletion or knockout of (i) an endogenous polynucleotide encoding the Pat1 protein, or a part thereof; or (ii) an expression control sequence that controls expression of said endogenous polynucleotide encoding the Pat1 protein.
3. The host cell of item 2, wherein said expression control sequence is selected from the group consisting of a promoter, a ribosomal binding site, transcriptional or translational start and stop sequences, an enhancer and an activator sequence.
4. The host cell of any one of items 1 to 3, wherein an endogenous gene encoding said Pat1 protein is knocked out.
5. The host cell of any one of items 1 to 4, wherein the expression cassette comprises one or more expression control sequences operably linked to said GOI, preferably comprising a promoter which is an inducible, de-repressible or otherwise regulatable promoter, or a constitutive promoter.
6. The host cell of any one of items 1 to 5, wherein the expression cassette comprises a promoter which is inducible in the presence of a growth-limiting amount of up to 1 g/L of a carbon source; and repressible in the presence of an excess amount of a carbon source that is higher than the growth-limiting amount.
7. The host cell of any one of items 1 to 6, wherein the POI is a therapeutic or diagnostic product, preferably a peptide, polypeptide or protein selected from the group consisting of an antigen-binding protein, an enzyme, a peptide, a protein antibiotic, a toxin fusion protein, a carbohydrate—protein conjugate, a structural protein, a regulatory protein, a vaccine antigen, a growth factor, a hormone, a cytokine, a process enzyme, and a metabolic enzyme.
8. The host cell of any one of items 1 to 7, which is
a) a yeast cell of a genus selected from the group consisting of Pichia, Hansenula, Komagataella, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromes marxianus, Yarrowia lipolytica or Hansenula polymorpha;
b) a cell of filamentous fungi, such as Aspergillus awamori or Trichoderma reesei;
c) a non-human primate, human, rodent or bovine cell, such as mouse myeloma (NSO)-cell lines, Chinese hamster ovary (CHO)-cell lines, HT1080, H₉, HepG2, MCF7, MDBK Jurkat, MDCK, NIH3T3, PC12, BHK (baby hamster kidney cell), VERO, SP2/0, YB2/0, YO, C127, L cell, COS, e.g., COS1 and COS7, QC1-3, HEK-293, VERO, PER.C6, HeLA, EB1, EB2, EB3, oncolytic or hybridoma-cell lines;
d) an insect cell, such as Sf9, Mimic™ Sf9, Sf21, High Five (BT1-TN-5B1-4), or BT1-Ea88 cells;
e) an algae cell, such as of the genus Amphora, Bacillariophyceae, Dunaliella, Chlorella, Chlamydomonas, Cyanophyta (cyanobacteria), Nannochloropsis, Spirulina, or Ochromonas); or
f) a plant cell, such as cells from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.
9. The host cell of any one of items 1 to 8, wherein the Pat1 protein is Pat1 protein comprising SEQ ID NO:1, or a Pat1 ortholog to SEQ ID NO:1, which is encoded by the host cell endogenous polynucleotide without said genetic modification to control expression of the Pat1 protein, preferably wherein the Pat1 protein or Pat1 ortholog originates from a host cell selected from the group consisting of any one of Komagataella phaffii, Komagataella pastoris, Komagataella pseudopastoris, Saccharomyces cerevisiae, Yarrowia lipolytica, Ogataea polymorpha, Schizosaccharomyces pombe, Trichoderma reesei, Kluyveromyces lactis, Aspergillus niger, Homo sapiens, or Cricetulu griseus host cells, in particular CHO cells.
10. The host cell of any one of items 1 to 9, wherein:
a) the host cell is a Komagataella phaffii host cell, and the Pat1 protein comprises at least 90% sequence identity to SEQ ID NO:1; or
b) the host cell is a Komagataella pastoris host cell, and the Pat1 protein comprises at least 90% sequence identity to SEQ ID NO:3; or
c) the host cell is a Saccharomyces cerevisiae host cell, and the Pat1 protein comprises at least 90% sequence identity to SEQ ID NO:4; or
d) the host cell is a Yarrowia lipolytica host cell, and the Pat1 protein comprises at least 90% sequence identity to SEQ ID NO:5; or
e) the host cell is an Ogataea polymorpha host cell, and the Pat1 protein comprises at least 90% sequence identity to SEQ ID NO:6; or
f) the host cell is a Schizosaccharomyces pombe host cell, and the Pat1 protein comprises at least 90% sequence identity to SEQ ID NO:7; or
g) the host cell is a Trichoderma reesei host cell, and the Pat1 protein comprises at least 90% sequence identity to SEQ ID NO:8 or
h) the host cell is a Kluyveromyces lactis host cell, and the Pat1 protein comprises at least 90% sequence identity to SEQ ID NO:9; or
i) the host cell is an Aspergillus niger host cell, and the Pat1 protein comprises at least 90% sequence identity to SEQ ID NO:10.
j) the host cell is a Homo sapiens host cell, and the Pat1 protein comprises at least 90% sequence identity to SEQ ID NO:11 or 12; or
k) the host cell is a Cricetulu griseus host cell, and the Pat1 protein comprises at least 90% sequence identity to any one of SEQ ID NO:13 to 17, or any one of SEQ ID NO:97-101.
11. The host cell of any one of items 1 to 10, which comprises an at least 1.2 fold increased specific productivity (μg/g cell dry mass (CDM), in particular yeast dry mass (YDM) for yeast cells) per hour and/or volumetric productivity (μg/L per hour) for said POI, compared to the host cell without said genetic modification to control expression of the Pat1 protein.
12. A method for producing a host cell of any one of items 1 to 10, comprising genetic engineering of a host cell to (i) introduce said recombinant expression cassette expressing said GOI, and to (ii) control expression of a Pat1 protein compared to an endogenous expression thereof.
13. A method of increasing the yield of producing a heterologous protein of interest (POI) in a host cell under carbon source limiting conditions, by engineering the host cell to control expression of a Pat1 protein in the host cell compared to an endogenous expression thereof.
14. The method of item 13, wherein the specific productivity (μg/g cell dry mass (CDM), in particular yeast dry mass (YDM) for yeast cells) per hour and/or volumetric productivity (μg/L per hour) for said POI is at least 1.2-fold increased, compared to the host cell without said genetic modification to control expression of the Pat1 protein.
15. The method of item 13 or 14, wherein the host cell is a host cell of any one of items 1 to 11.
16. A method for producing a protein of interest (POI) in a host cell culture, by culturing the host cell of any one of items 1 to 11 under conditions to produce said POI.
17. A method of item 15, wherein the host cell is cultured under carbon source limiting conditions to produce said POI, preferably in the presence of a growth-limiting amount of a carbon source which is up to 1 g/L.
18. A method of item 16 or 17, wherein the host cell is cultured in a culture medium under conditions to secrete said POI into the host cell culture, and the POI is recovered from the host cell culture.
19. The method of any one of items 16 to 18, wherein said expression of a Pat1 protein is reduced to a level that increases the host cell's specific productivity for said POI (μg/g cell dry mass (CDM), in particular yeast dry mass (YDM) for yeast cells) per hour and/or volumetric productivity for said POI (μg/L per hour).
20. A pat1 knockout eukaryotic host cell producing a protein of interest (POI) at an at least 1.2-fold increased specific productivity (μg/g cell dry mass (CDM), in particular yeast dry mass (YDM) for yeast cells) per hour and/or volumetric productivity (μg/L per hour) compared to the host cell without said pat1 knockout, preferably under carbon source limiting conditions to produce said POI.
21. The host cell of item 20, wherein the pat1 gene encodes a Pat1 protein comprising SEQ ID NO:1 or a Pat1 ortholog to SEQ ID NO:1, preferably wherein the Pat1 protein or Pat1 ortholog originates from a host cell selected from the group consisting of any one of Komagataella phaffii, Komagataella pastoris, Komagataella pseudopastoris, Saccharomyces cerevisiae, Yarrowia lipolytica, Ogataea polymorpha, Schizosaccharomyces pombe, Trichoderma reesei, Kluyveromyces lactis, Aspergillus niger, Homo sapiens, or Cricetulu griseus host cells, in particular CHO cells.
22. The host cell of item 20 or 21, wherein
a) the host cell is a Komagataella phaffii host cell, and the Pat1 protein comprises at least 90% sequence identity to SEQ ID NO:1; or
b) the host cell is a Komagataella pastoris host cell, and the Pat1 protein comprises at least 90% sequence identity to SEQ ID NO:3; or
c) the host cell is a Saccharomyces cerevisiae host cell, and the Pat1 protein comprises at least 90% sequence identity to SEQ ID NO:4; or
d) the host cell is a Yarrowia lipolytica host cell, and the Pat1 protein comprises at least 90% sequence identity to SEQ ID NO:5; or
e) the host cell is an Ogataea polymorpha host cell, and the Pat1 protein comprises at least 90% sequence identity to SEQ ID NO:6; or
f) the host cell is a Schizosaccharomyces pombe host cell, and the Pat1 protein comprises at least 90% sequence identity to SEQ ID NO:7; or
g) the host cell is a Trichoderma reesei host cell, and the Pat1 protein comprises at least 90% sequence identity to SEQ ID NO:8 or
h) the host cell is a Kluyveromyces lactis host cell, and the Pat1 protein comprises at least 90% sequence identity to SEQ ID NO:9; or
i) the host cell is an Aspergillus niger host cell, and the Pat1 protein comprises at least 90% sequence identity to SEQ ID NO:10.
j) the host cell is a Homo sapiens host cell, and the Pat1 protein comprises at least 90% sequence identity to SEQ ID NO:11 or 12; or
k) the host cell is a Cricetulu griseus host cell, and the Pat1 protein comprises at least 90% sequence identity to any one of SEQ ID NO:13 to 17, or any one of SEQ ID NO:97-101.

The Examples which follow are set forth to aid in the understanding of the invention but are not intended to, and should not be construed to limit the scope of the invention in any way. The Examples do not include detailed descriptions of conventional methods and devices. Such methods and devices are well known to those of ordinary skill in the art.

### EXAMPLES

The examples below demonstrate that deletion of the decapping factor PAT1 leads to a global higher translational activity of P. pastoris enabling increased productivity of recombinant proteins.

### Example 1: Construction of Production and Deletion Strains

### a) Host Strains and Expression Vectors.

P. pastoris strains CBS7435 or CBS2612 (CBS-KNAW Fungal Biodiversity Centre, Centraalbureau voor Schimmelcultures, Utrecht, The Netherlands) were used as background strains. To test the impact of the deletions, different secretory model proteins were used as reporters. The deletions were either made to the non-expressing P. pastoris background strains (Example 1b), which were then transformed with expression cassettes for the recombinant genes of interest (GOI). Alternatively, the deletions were generated in established P. pastoris production strains e.g., the strain CBS2612_P_{G1-3}_vHH #4 (described in WO2020144313A1), which expresses the nanobody vHH under control of the glucose-limit inducible promoter P_{G1-3}.

To have additional testing strains available, the expression cassette for the nanobody, P_{G1-3}_vHH, and human serum albumin, P_{G1-3}_HSA, were transformed into a generated CBS7435_Apat1 strain (see Example 1b) as described in WO2020144313A1.

The used pPUZZLE plasmids (Stadlmayr et al. 2010. J Biotechnol. 2010 December; 150(4):519-29. doi:10.1016/j.jbiotec.2010.09.957.) pPM1aZ30_P_{G1-3}_vHH and pPM1aZ30_P_{G1-3}_HSA, each contained the Zeocin resistance cassette, the gene of interest (bivalent nanobody vHH or human serum albumin HSA, both codon optimized for expression in P. pastoris by commercial suppliers), a leader sequence (S. cerevisiae alpha-mating factor pre-pro leader for vHH and the native human leader for HSA), the S. cerevisiae CYC1 transcription terminator, as well as a locus for integration into the P. pastoris genome (3′AOX1 region). The coding sequence of both genes of interest (codon-optimized and synthesized by external providers) can be found in the sequence file.

### b) Generation of Deletion Strains.

To generate the Apat1 mutant strain, the gene PP7435_Chr2-0400 (PAT1) was deleted by a split-marker cassette method similar to the adapted method for P. pastoris (Gasser et al. 2013. Future Microbiol. February; 8(2):191-208. doi: 10.2217/fmb.12.133.) and as described in Heistinger et al. 2018 (Mol Cell Biol. 2018 Jan. 15; 38(2). doi: 10.1128/MCB.00398-17).

Briefly, two 1.0 kb regions located up- and downstream of the ORF were amplified from purified P. pastoris CBS2612 genomic DNA, using primers A_fwd and A_rev as well as D_fwd and D_rev, respectively (Table 1). These primers contained fusion sites A to D for smooth integration of the generated fragments into a GoldenPiCS BB3 plasmid. The GoldenPiCS vector system is described in Prielhofer et al. 2017 (BMC Systems Biol. 11, 123, doi: 10.1186/s12918-017-0492-3). Another set of primers with added fusion sites, B-C_NTC_FsB_fwd and B-C_NTC_FsC_rev, was used to amplify the NatMX resistance cassette flanked by IoxP sites (see Table 2) to enable marker recycling with Cre recombinase. A previously generated plasmid by Heistinger et al. 2018 was used as template.

Golden Gate assembly of the generated fragments A, B-C and D into BB3aK_FsA_FsD as well as subsequent transformation into E. coli, purification and sequence control was performed according to the previously described protocols (Prielhofer et al. 2017; WO2022069613A1).

The generated plasmids were then used as template to amplify fragments AB and CD by PCR. Primers for both fragments were designed to generate a 400 bp overlapping region within the NatMX cassette (Table 1). Both fragments were transformed into P. pastoris at the same time by electroporation using a standard protocol (Gasser et al. 2013). The transformation resulted in full replacement of each gene's open reading frame (ORF) with the NatMX cassette.

Selection of positive transformants was done on selective YPD-agar plates (per liter: 10 g yeast extract, 20 g peptone, 20 g glucose, 20 g agar-agar) containing 100 μg mL⁻¹ Nourseotricin. Successful deletion was verified by colony PCR with primers located inside of the targeted genes (Det_fwd and Det_rev, Table 1) and subsequent gel electrophoresis. As the primers were located within the targeted genes, no visible band after the PCR meant the deletion was successful. Any clone showing such a result was restreaked and controlled at least two more times by colony PCR to confirm this result. Appearance of a band in any control resulted in immediate exclusion of said clone.

TABLE 1
Primers used for generation of deletion cassettes for the gene PAT1 and
detection thereof by PCR.

| Name | Sequence |
|---|---|
| A_FsA_pat1_fwd | GTAGGGTCTCCGATCGGAGCCGATTGACGA |
| | SEQ ID NO: 60 |
| A_FsB_pat1_rev | GATGGGTCTCGCCGGGGTTGATTTCTATTGTCACAGG |
| | SEQ ID NO: 61 |
| B-C_NTC_FsB_fwd | GATAGGTCTCCCCGGGTACGCTGCAGGTCGACAAC |
| | SEQ ID NO: 62 |
| B-C_NTC_FsC_rev | GATAGGTCTCCAATTAGTGGATCTGATATCACCTA |
| | SEQ ID NO: 63 |
| D_FsC_pat1_fwd | GTTCGGTCTCCAATTGTCTCGATGTTCAAATTCTG |
| | SEQ ID NO: 64 |
| D_FsD_pat1_rev | GATCGGTCTCGAGCTCGGGTCTGGCAATCT |
| | SEQ ID NO: 65 |
| PAT1_ABAmp_fwd | CAGAGATCAATACCAAATACCTG |
| | SEQ ID NO: 66 |
| PAT1_CDAmp_rev | CAGCCAAGAATGGTGTG |
| | SEQ ID NO: 67 |
| ABAmp_rev | TGCGTTGACGTTGGTGAC |
| | SEQ ID NO: 68 |
| CDAmp_fwd | GTCCTTCACCACCGACACC |
| | SEQ ID NO: 69 |
| PAT1_Det_fwd | GGCGATGGTGAGAAC |
| | SEQ ID NO: 70 |
| PAT1_Det_rev | CCGAACATTCATCCTG |
| | SEQ ID NO: 71 |

TABLE 2
The cassette of NatMX for the split marker method. The two loxP sites
are underlined.

| | |
|---|---|
| NatMX | GTACGCTGCAGGTCGACAACCCTTAATATAACTTCGTATAATGTATGCTATACGAAGTTATTA |
| expression | GGTCTAGATCGGTACCGACATGGAGGCCCAGAATACCCTCCTTGACAGTCTTGACGTGCGCAG |
| cassette | CTCAGGGGCATGATGTGACTGTCGCCCGTACATTTAGCCCATACATCCCCATGTATAATCATT |
| with loxP | TGCATCCATACATTTTGATGGCCGCACGGCGCGAAGCAAAAATTACGGCTCCTCGCTGCAGAC |
| sites | CTGCGAGCAGGGAAACGCTCCCCTCACAGACGCGTTGAATTGTCCCCACGCCGCGCCCCTGTA |
| | GAGAAATATAAAAGGTTAGGATTTGCCACTGAGGTTCTTCTTTCATATACTTCCTTTTAAAAT |
| | CTTGCTAGGATACAGTTCTCACATCACATCCGAACATAAACAACCATGGGTACCACTCTTGAC |
| | GACACGGCTTACCGGTACCGCACCAGTGTCCCGGGGGACGCCGAGGCCATCGAGGCACTGGAT |
| | GGGTCCTTCACCACCGACACCGTGTTCCGCGTCACCGCCACCGGGGACGGCTTCACCCTGCGG |
| | GAGGTGCCGGTGGACCCGCCCCTGACCAAGGTGTTCCCCGACGACGAATCGGACGACGAATCG |
| | GACGACGGGGAGGACGGCGACCCGGACTCCCGGACGTTCGTCGCGTACGGGGACGACGGCGAC |
| | CTGGCGGGCTTCGTGGTCGTCTCGTACTCCGGCTGGAACCGCCGGCTGACCGTCGAGGACATC |
| | GAGGTCGCCCCGGAGCACCGGGGGCACGGGGTCGGGCGCGCGTTGATGGGGCTCGCGACGGAG |
| | TTCGCCCGCGAGCGGGGCGCCGGGCACCTCTGGCTGGAGGTCACCAACGTCAACGCACCGGCG |
| | ATCCACGCGTACCGGCGGATGGGGTTCACCCTCTGCGGCCTGGACACCGCCCTGTACGACGGC |
| | ACCGCCTCGGACGGCGAGCAGGCGCTCTACATGAGCATGCCCTGCCCCTAATCAGTACTGACA |
| | ATAAAAAGATTCTTGTTTTCAAGAACTTGTCATTTGTATAGTTTTTTTATATTGTAGTTGTTC |
| | TATTTTAATCAAATGTTAGCGTGATTTATATTTTTTTTCGCCTCGACATCATCTGCCCAGATG |
| | CGAAGTTAAGTGCGCAGAAAGTAATATCATGCGTCAATCGTATGTGAATGCTGGTCGCTATAC |
| | TGGTACCATTCGAGAACCCTTAATATAACTTCGTATAATGTATGCTATACGAAGTTATTAGGT |
| | GATATCAGATCCACT (SEQ ID NO: 72) |

To excise the NatMX selection marker cassette from the genome based on Cre-IoxP recombination, each strain was transformed transiently with the Cre-recombinase expression plasmid, pKTAC_CreshphMX4, by electroporation as described in Gasser et al, 2013 and WO2020144313A1.

As this plasmid contains a Hygromycin resistance cassette, selection of positive transformants was done on YPD-agar containing 200 μg mL⁻¹ Hygromycin. Hygromycin resistant clones were subsequently restreaked on YPD-agar (without Hygromycin to promote loss of pKTAC_Cre_hphMX4). To verify loss of the plasmid as well as successful excision, obtained clones were cultivated in three parallel cultures in YPD media (per liter: 10 g yeast extract, 20 g peptone, 20 g glucose) supplemented with either 100 μg mL⁻¹ Nourseotricin, 200 μg mL⁻¹ Hygromycin or no antibiotic for 30 h at 25° C. Cultures which did not show growth in either antibiotic, but growth without antibiotics, were used for the subsequent experiments. In Table 3 an overview of generated strains is provided.

TABLE 3
Names, genotypes and source of the strains used in this work.

| | | | Model- | |
|---|---|---|---|---|
| Strain name | Modifications/Expression cassettes | Resistance | protein | Source |
| CBS2612_ | CBS2612 + pPM1aZ30_P_{G1-3}-vHH #4 | ZeoR | vHH | parent strain; |
| vHH#4 | | | | WO2020144313A1 |
| CBS2612_ | CBS2612 + pPM1aZ30_P_{G1-3}-vHH #4 | ZeoR | vHH | generated |
| vHH_Δpat1 | pat1A::loxP-natMX-loxP | NatMX | | |
| CBS2612_ | CBS2612 + pPM1aZ30_PG₁₋₃-vHH #4 | ZeoR | vHH | generated |
| vHH_Δpat1lx | pat1Δ::loxP | | | |
| CBS7435_ | CBS7435 + pPM1aZ30_PG₁₋₃-vHH #5 | ZeoR | vHH | reference strain; |
| vHH#5 | | | | WO2022069613A1 |
| CBS7435_ | pat1Δ::loxP-natMX-loxP | NatMX | none | generated |
| Δpat1 | | | | |
| CBS7435_ | pat1Δ::loxP | none | none | generated |
| Δpat1lx | | | | |
| CBS7435_ | pat1Δ::loxP #47/#50 + | ZeoR | vHH | generated |
| Δpat1lx_ | pPM1aZ30_P_{G1-3}-vHH | | | |
| vHH | | | | |
| CBS7435_ | CBS7435 + pPM1aZ30_P_{G1-3}-HSA | ZeoR | HSA | reference strain; |
| HSA | | | | generated |
| CBS7435_ | pat1Δ::loxP #25/#47/#50/#86 + | ZeoR | HSA | generated |
| Δpat1_HSA | pPM1aZ30_P_{G1-3}-HSA | NatMX | | |
| CBS7435_ | CBS7435_P_{G1-3}-VHH #5 + 5′-P_{MDH3}- | ZeoR | vHH | reference strain; |
| vHH_C3b | TIF4632-RPS3tt-P_{POR1}-TIF2-RPS17Btt- | NatMX | | WO2022069613A1 |
| | P_{PDC1}-CDC33-IDP1tt-P_{GPM1}-PAB1- | | | |
| | RPS25tt-natMX-3′ integrated in RGI1 | | | |
| | locus | | | |
| CBS7435_ | pat1Δ::loxP #47/#50 + | ZeoR | vHH | generated |
| Δpat1lx_ | pPM1aZ30_P_{G1-3}-VHH #+ | NatMX | | |
| vHH_C3b | 5′-P_{MDH3}-TIF4632-RPS3tt-P_{POR1}-TIF2- | | | |
| | RPS17Btt-P_{PDC1}-CDC33-IDP1tt-P_{GPM1}- | | | |
| | PAB1-RPS25tt-natMX-3′ integrated in | | | |
| | RGI1 locus | | | |

### c) Generation of Overexpression Strain

For further comparisons, the generated pat1Δ strains (see Table 3) were transformed with the overexpression cassette C3b described in WO2022069613A1. By this, the genes T/F4632 (PP7435_Chr1-0352), TIF2b (PP7435_Chr3-0610), CDC33 (PP7435_Chr3-0197) and PAB1 (PP7435_Chr2-1212) were overexpressed in the cells under control of constitutive promoters. The cloning procedure, protocol for confirmation of successful integration and the used promoters are described in WO2022069613A1.

### Example 2: Effects of Decapping Factor Deletion on Recombinant Protein Production in Small Scale Screenings

To determine the effect of decapping factor deletion on recombinant protein secretion, engineered mutant strains were cultivated in glucose limiting conditions to induce the used P_{G}-promoter for the GOI, P_{G1-3} (Prielhofer et al. 2013. Microb Cell Fact 12, 5). The engineering of the P. pastoris parent/reference strains were done as described in Example 1. Verified single colonies were used for cultivation in small scale (screening procedure), which simulated a fed-batch cultivation. The recombinant protein secreted into the supernatant was quantified and the titers and yields of the different engineered strains were compared to the parent strain.

a) Screening of Engineered P. pastoris Strains Under Glucose-Limit.

Media: synthetic screening medium ASMv6 per liter: 6.30 g (NH₄)₂HPO₄, 0.8 g (NH₄)₂SO₄, 0.49 MgSO₄*7H₂O, 2.64 g KCl, 0.0535 g CaCl₂)*2H₂O, 22.0 g citric acid monohydrate, 1470 μL PTM₀ trace salt stock solution, 20 mL NH₄OH (25%), 4 mL Biotin (0.1 g L⁻¹). Solid KOH was added to set the pH to 6.4-6.6.

PTM₀ trace salt stock solution per liter: 5.0 mL H₂SO₄ (95-98%), 65.0 g FeSO₄*7H₂O, 20 g ZnCl₂, 6.00 g CuSO₄*5H₂O, 3.36 g MnSO₄*H₂O, 0.82 g CoCl₂*6H₂O, 0.20 g Na₂MoO₄*2H₂O, 0.08 g NaI, 0.02 g H₃BO₃ For the small-scale screening, single colonies were inoculated in 2 mL liquid YPG medium (per liter: 20 g peptone, 10 g yeast extract, 12.6 g glycerol 100%, pH 7.4-7.6) containing 50 μg mL⁻¹ Zeocin and 100 μg mL⁻¹ nourseothricin (if appropriate). Additionally, on each plate the parent/reference strain was cultivated in quadruplicate for comparison. This pre-culture was grown for approximately 24 h at 25° C. in 24-DWP (deep well plates) at 280 rpm. The pre-cultures were then used to inoculate 2 mL of synthetic screening medium ASMv6 to a starting-OD₆₀₀ of 8. The media contained 50 g L⁻¹ polysaccharide (EnPump200 polysaccharide, Enpresso) and 0.3% of glucose-releasing enzyme (Reagent A, Enpresso) as carbon source. Cultivation conditions were similar to pre-culture conditions. After a time indicated in each section (72-76 h), 1 mL of cell suspension was transferred to a pre-weighted 1.5 mL centrifugation tube and centrifuged at 16,000 g for 5 min at room temperature. Supernatants were carefully transferred to a new vial and stored at −20° C. until further use. Centrifugation tubes containing the pellets were weighted again to determine the wet cell weight (WCW). Quantification of the recombinant secreted protein in the supernatant was done by microfluidic capillary electrophoresis as described below.

b) Quantification of Secreted Recombinant Protein by Microfluidic Capillary Electrophoresis (mCE).

The ‘LabChip GX/GXII System’ (PerkinElmer) was used for quantitative analysis of secreted protein titer in culture supernatants. The consumables ‘Protein Express Lab Chip’ (760499, PerkinElmer) and ‘Protein Express Reagent Kit’ (CLS960008, PerkinElmer) were used. Chip and sample preparation were done according to the manufacturer's recommendations. A brief description of the procedure is given below.

Chip preparation: After the reagents came to room temperature 520 and 280 μL of Protein Express Gel Matrix were transferred to spin filters. 20 μL of Protein Express Dye solution was added to the 520 μL Gel Matrix containing spin filter. After briefly vortexing the dye containing spin filter in the inverted orientation, both spin filters were centrifuged at 9300 g for 10 minutes. To wash the chip, 120 μL Milli-Q® water were added to all active chip wells and the chip was subjected to the instruments washing program. After two further rinsing steps with Milli-Q® water, remaining fluids were fully aspirated and appropriate amounts of the filtered Gel Matrix solutions as well as the Protein Express Lower Marker solution were added to the appropriate chip wells.

Sample and ladder preparation: For sample preparation 6 μL sample were mixed with 21 μL of sample buffer in a 96-microtiter plate. Samples were denatured at 100° C. for 5 min and centrifuged at 1,200 g for 2 min. Subsequently, 105 μL of Milli-Q® water were added. Sample solutions were briefly mixed by pipetting and centrifuged again at 1,200 g for 2 min before measurement. To prepare the ladder 12 μL of Protein Express Ladder were denatured at 100° C. for 5 min in a PCR tube. Subsequently, 120 μL of Milli-Q® water were added and the ladder solution was briefly vortexed before spinning the tube for 15 seconds in a minicentrifuge and starting the measurement.

Quantitation was done by employing the LabChip software provided by the manufacturer and comparison against BSA standards.

### Example 3: Determining Effects of Deletion of mRNA Decapping Machinery Components on Recombinant Protein Production

### a) Effect of PAT1 Deletion in a Recombinant Protein Producing Strain

First, effects of PAT1 deletion in a strain already established for high level protein production were analysed. For this, the P. pastoris strain generated in WO2020144313A1, CBS2612_vHH #4, which secretes high levels of the nanobody vHH was engineered as described in Example 1, by deleting the ORFs coding for Pat1. Deletion was achieved by replacing the ORFs with a NatMX resistance cassette using the split marker cassette approach. A successful clone was identified (#18).

The engineered strain was then analysed in small scale screenings as described in Example 2. From each pre-culture, two wells with synthetic screening media were inoculated in parallel in the main culture. One of each duplicate was harvested after 50 h, the other after 76 h of cultivation to make calculation of specific productivities (qP) possible. In the first round of screenings, the Apat1 deletion strain was cultivated, and growth as well as vHH secretion were compared to the parent CBS2612_vHH #4 cultivated in quadruplicates on the same DWP. In the second round of screening, CBS2612_vHH Apat1 #18 was cultivated in quadruplicates. Table 4 shows the obtained product titers and WCW obtained after 76 h of cultivation. The fold change (FC) of the vHH yield (titer divided by WCW) compared to the parent strain is also shown for the 76 h data point. Additionally, specific productivity was determined between 50 h and 76 h of cultivation by calculating the increase in protein titer per average biomass in this interval and time passed.

TABLE 4
Effect of deletion of PAT1 on recombinant protein production in the established nanobody secreting
clone CBS2612_vHH#4 in small scale screenings. Samples for the titer and WCW were taken after
76 h of cultivation. FC depicts the fold change of the WCW, secreted vHH Titer or secreted vHH yield
(titer/WCW) between the generated strains and the parent strain after 76 h of cultivation. The specific
productivities (qP) were determined using the values measured for the 50 h and the 76 h samples.

| | 1^{st} screening | | | | 2^{nd} screening | | | |
|---|---|---|---|---|---|---|---|---|
| | | FC | | qP | | FC | FC | qP |
| | FC titer | WCW | FC | [mg g⁻¹ | FC titer | WCW | vHH | [mg g⁻¹ |
| Strain/Clone | [mg L⁻¹] | [g L⁻¹] | vHHyield | h⁻¹] | [mg L⁻¹] | [g L⁻¹] | yield | h⁻¹] |
| CBS2612_vHH#4 | Ref. | Ref. | Ref. | 0.005 | Ref. | Ref. | Ref. | 0.010 |
| CBS2612_vHH_Δpat1#18 | 6.2 | 1.5 | 4.1 | 0.039 | 3.0 ± 0.1 | 1.4 ± 0.0 | 2.2 ± 0.0 | 0.050 |

The results showed that deletion of PAT1 has a clear positive effect. The Δpat1 strain produced 2 to 4-fold higher yields and, in the last day of cultivation, showed more than 5-fold higher specific productivity compared to the parent strain. Seemingly, productivity of Δpat1 increases even more in later stages of the screening.

b) Confirming the Effect Decapping Factor Deletion on Recombinant Protein production by Introducing POI Expression Cassettes in the PAT1 Deletion background

To confirm the effects seen for Δpat1 with more clones and in a different P. pastoris background, PAT1 was deleted in the CBS7435 wild type by the split marker cassette approach as described in Example 1. After identification of multiple clones with successful deletion and recycling of the marker with Cre recombinase, two clones (CBS7435_Δpat1lx #47 and #50) were chosen for the further experiments. These two deletion clones were transformed with the P_{G1-3}_vHH expression cassette (described in Example 1) and, after one round of re-streaking, used directly in a small-scale screening (described in Example 2). In this case, a reference clone was used for comparison. For this purpose, CBS7435_P_{G1-3}_vHH #5, generated and described in WO2022069613A1, was chosen. Complete samples were taken after 72 h of cultivation, while after 49 h 200 μL were taken for an additional protein titer measurement of the supernatant. Results of this screening can be found in Table 5. Clones with potential integration of multiple copies of the GOI expression cassette were excluded from the analysis. For comparative purposes, also the strains from Example 3a were cultivated in the same screening procedure. Table 5 shows names of the used strains, the number of clones or replicates used for the calculations as well as Titer, WCW and FC of the vHH yield after 72 h of cultivation. Additionally, productivities (difference of titer divided by time passed) are shown for the time interval between 49 h and 72 h of cultivation.

TABLE 5
Recombinant nanobody production in wild type and PAT1 deletion backgrounds in small
scale screenings. Samples for the titer and WCW were taken after 72 h of cultivation.
FC of yield depicts the fold change of the yield (titer/WCW) between the generated strains
and the reference strain at the end of the cultivation. The productivities (p) were determined
with the values measured for the 49 h and the 72 h samples.

| | Nr. of clones/ | Titer | WCW | FC vHH | p |
|---|---|---|---|---|---|
| Strain | replicates | [mg L⁻¹] | [g L⁻¹] | yield | [mg h⁻¹] |
| CBS2612_vHH#4 | 5 replicates | 44.4 ± 3.6 | 58.6 ± 0.9 | Ref. | 0.36 ± 0.03 |
| CBS2612_vHH_Δpat1#18 | 6 replicates | 164.0 ± 2.0 | 79.2 ± 0.7 | 2.7 ± 0.0 | 3.93 ± 0.18 |
| CBS7435_vHH#5 | 6 replicates | 17.9 ± 1.3 | 58.6 ± 1.4 | Ref. | 0.17 ± 0.03 |
| CBS7435_Δpat1lx#47_vHH | 7 clones | 134.1 ± 11.3 | 85.4 ± 0.9 | 5.2 ± 0.4 | 3.49 ± 0.24 |
| CBS7435_Δpat1lx#50_vHH | 8 clones | 149.2 ± 13.0 | 85.7 ± 1.6 | 5.7 ± 0.5 | 3.96 ± 0.33 |

The results further confirmed the effects achieved by decapping factor deletion seen in Example 3a. Deletion of PAT1 in the CBS7435 strain background leads to 5 to 6-fold higher vHH yields compared to the reference strain. Again, also productivity was immensely increased (up to 23-fold) in the later stage of the screening procedure. The CBS2612 strains behaved similar to the screenings shown in Table 4, meaning the yield was increased by about 3-fold in Δpat1. This comparison further verifies that the effect of the PAT1 deletion is independent of the order of the workflow of generating production strains, and independent of the strain background.

c) Effect of PAT1 Deletion on Different Recombinant Proteins—Small-Scale Screenings with HSA as Model Protein.

To test if the effects seen in the previous examples only apply to nanobody production or the deletion can be used to improve production of different proteins, recombinant HSA producing strains were generated and tested as well.

First, effects were determined in small scale screenings. For this, four clones (#25, #47, #50 and #86) of the deletion strain CBS7435_Δpat1/x (described in Example 3b) were used as host strains for the P_{G1-3}_HSA expression cassette. The expression cassette as well as the transformation protocol are described in Example 1. To also have a reference strain, the CBS7435 wild type was transformed with the P_{G1-3}_HSA expression cassette at the same time. The engineered strains were screened with the protocol described in Example 2. Samples were taken after 72 h of cultivation. Due to the number of samples, multiple 24-DWP needed to be used for this screening. To ensure the plates are comparable, six wells containing different reference strain clones (#1-6) were inoculated from the same pre-culture wells into the different main culture plates. Table 6 shows the results of these screenings. With integration of the protein expression cassette into the P. pastoris genome some clones obtained multiple copies of P_{G1-3}_HSA. As results of such clones were not comparable, all average values shown in Table 7 are corrected for these outliers.

TABLE 6
Recombinant HSA production in wild type and Δpat1 background strains in small scale screenings.
Samples for the titer and WCW were taken after 72 h of cultivation. FC HSA yield depicts the fold
change of the yield (titer/WCW) between the Δpat1 background strains and the reference strain.
Additionally, the number of clones screened as well as the number usedfor calculations
(which excludes outliers) is shown (screened/used).

| Strain | Nr. of | Titer | WCW | FC HSA |
|---|---|---|---|---|
| | clones | [mg L⁻¹] | [g L⁻¹] | yield |
| CBS7435_HSA | 6/5 | 14.3 ± 2.7 | 49.7 ± 1.5 | Ref. |
| CBS7435_ | 6/5 | 97.0 ± 22.0 | 89.5 ± 1.9 | 3.8 ± 0.8 |
| Δpat1#25_vHH | | | | |
| CBS7435_ | 11/8 | 100.6 ± 2.8 | 88.7 ± 1.6 | 3.9 ± 0.1 |
| Δpat1#47_vHH | | | | |
| CBS7435_ | 11/9 | 100.2 + 6.8 | 87.8 ± 2.2 | 4.0 ± 0.2 |
| Δpat1#50_vHH | | | | |
| CBS7435_ | 7/7 | 92.2 ± 8.7 | 89.6 ± 3.4 | 3.6 ± 0.4 |
| Δpat1#86_vHH | | | | |

The results obtained for HSA production corresponded perfectly with the results of vHH production. Again, Δpat1 could increase the obtained recombinant protein yield 4-fold. Interestingly, also biomass was highly affected by Δpat1. These results indicate that the deletion of the decapping factor provides a universal mean to improve recombinant protein production and secretion.

### d) Gene Copy Number Determination for the GOI Expression Cassette

As expression strength is often correlated to the number of expression cassettes integrated into the P. pastoris genome, gene copy number (GCN) needed to be determined for selected clones before continuing further. This ensured that the differences seen between reference and deletion strains indeed stems from the genetic modifications.

For the determination, genomic DNA was isolated from pelleted cells using the Wizard® Genomic DNA Purification Kit (Promega Corporation, Cat. No. A1120). Then, gene copy numbers were determined using quantitative real-time PCR (qPCR). For this, the Blue S'Green qPCR Kit (Biozym) was used. The Blue S'Green qPCR master mix was mixed with primers and samples and applied for real time analysis in a real-time PCR cycler (Rotor Gene, Qiagen). A list of used primers is shown in Table 7. All samples were analysed in triplicates. The Rotor Gene software was used for data analysis. As a calibrator, the ACT1 gene was used.

TABLE 7
qPCR Primers used for HSA expression cassette GCN determination.
ACT1 was used as calibrator.

| | | | Product |
|---|---|---|---|
| Name | Target | Sequence | Length |
| qPCR_PpACT1_fwd | ACT1 | CCTGAGGCTTTGTTCCACCCATCT | 148 bp |
| | | SEQ ID NO: 73 | |
| qPCR_PpACT1_rev | ACT1 | GGAACATAGTAGTACCACCGGACATAACGA | |
| | | SEQ ID NO: 74 | |
| qPCR_HSA_fwd | HSA | AGACTTTCACTTTCCACGCT | 153 bp |
| | | SEQ ID NO: 75 | |
| qPCR_HSA_rev | HSA | CAACGAAAGCAGCGAAGTC | |
| | | SEQ ID NO: 76 | |

The results showed that clones with much higher titers than others of the same strain—which were excluded as outliers beforehand—had up to seven copies of the protein expression cassette integrated in their genome. However, the clones used for the calculations in Table 6 only had obtained a single copy. Two reference clones and two Δpat1 clones were chosen to be used in a round of fed-batch cultivations.

### Example 4: Fed-Batch Cultivations of PAT1 Deletion Strains

To further validate the observations made in the screenings, fed-batch cultivations similar to standard production processes for the respective promoter system were done with Δpat1.

### a) Fed-Batch Cultivation Procedure

Media: PTM₀ trace salt stock solution per liter:
5.0 mL H₂SO₄ (95-98%), 65.0 g FeSO₄*7H₂O, 20 g ZnCl₂, 6.00 g CuSO₄*5H₂O, 3.0 g MnSO₄*H₂O, 0.5 g CoCI2*6H₂O, 0.20 g Na₂MoO₄*2H₂O, 0.08 g NaI, 0.02 g H₃BO₃

Glycerol Batch medium contained per liter:
2 g Citric acid monohydrate (C₆H₈O₇*H₂O), 45 g Glycerol, 12.6 g (NH₄)₂HPO₄, 0.5 g MgSO₄*7H₂O, 0.9 g KCl, 0.022 g CaCl₂*2H₂O, 13.2 mL biotin stock solution (0.1 g L⁻¹) and 4.6 mL PTMO trace salts stock solution. HCl (conc.) was added to set the pH to 5.

Glucose feed media contained per liter:
495 g glucose monohydrate, 4.6 g MgSO₄*7H₂O, 8.4 g KCl, 0.28 g CaCl₂*2H₂O, 23.6 mL biotin stock solution (0.1 g L⁻¹) and 10.1 mL PTMO trace salts stock solution.

In each round of fed-batch cultivations the parent/reference strain and the Δpat1 strain were cultivated in duplicates in 1 L benchtop bioreactors (SR07000DLS; Dasgip, Eppendorf, Germany). This system allows for usage of four parallel bioreactors. For pre-cultures 100 mL YPG media containing 50 μg mL⁻¹ Zeocin and 100 μg mL-1 nourseothricin (if appropriate) in a 1 L shake flask were inoculated with a 1.0 mL cryostock and incubated for around 24 h at 180 rpm and 25° C. Batch cultures were operated at a working volume of 0.25 L and were inoculated to a starting OD₆₀₀ of 1.5. Glycerol batch media composition is given above. During the entire process the temperature was controlled at 30° C., the DO was kept at 30% by automated adjustment of stirrer speed (between 400 and 1200 rpm) and air flow (between 9.5 and 30 sL h⁻¹), and the pH was regulated to be at 5.0 by automated addition of 12.5% NH₄OH. After a sudden spike in DO, indicating batch-end (BE), a linear incremental glucose feed (media composition detailed above) resulting in fast initial growth rates (p) followed by an extended phase of gradually decreasing p was applied. The linear increase of the feed was set to follow the equation: F[mL h⁻¹]=0.1431*t+2.0499.

Yeast dry mass (YDM) and secreted recombinant proteins were analysed at various time points throughout the process (shown in Tables 8 and 9). For YDM analysis 1 mL of culture broth was transferred to a 2 mL pre-dried (at 105° C. for at least 24 h) and pre-weighted centrifugation tube. After centrifugation at 16,000 g and 4° C. for 5 min the supernatant was carefully transferred to a fresh vial and stored at −20° C. until further use. Cell pellets were washed twice with 0.1 M HCl and dried at 105° C. for at least 24 h before the weight was measured again. YDM was analysed in triplicates.

Supernatants were analyzed in duplicate by microfluidic capillary electrophoresis (GXII, Perkin-Elmer) as described in Example 2b.

### b) Determination of Overall Translation Activity in the Mutant Strain.

The measurement of overall translation activity with O-propargyl labelled puromycin (OPP) was done as described in Staudacher et al. 2021 (BMC Microbiol. 21(1):120. doi: 10.1186/s12866-021-02185-3). Briefly, cells from selected fed-batch cultivation samples were pipetted in duplicate into a 96-well microtiter plate with an end-OD₆₀₀ of 0.4 in 90 μL “Incubation Solution”. The “Incubation Solution” consisted of ASMv6 media (see Example 2) supplemented with 0.6 mM O-propargyl puromycin (Jena Bioscience, NU-931-05), dissolved in 10% DMSO and PBS (2 mM KH₂PO₄, 10 mM Na₂HPO₄·2 H₂O, 2.7 mM g KCl, 8 mM NaCl, pH 7.4), and 1.5 g L⁻¹ Imipramine. The suspension was incubated for 2 h at 25° C. on a shaker, transferred into ice-cold Eppendorf tubes and centrifuged at 16,000 g for 5 min at 4° C. After washing the pelleted cells with 120 μL PBS, the again pelleted cells were fixed with 1 mL of ice-cold 70% ethanol. These fixed samples were stored between 1 day and 2 weeks at 4° C.

After collection and storage of all samples the click chemistry reaction was done. For this, the fixed samples were harvested by centrifugation at 16,000 g and 4° C. for 5 min. The pellet was transferred to a 96-well microtiter plate and washed with 100 μL “Click Chemistry Buffer” (115 mM Tris/HCl pH=8.5, 0.1% Triton X-100). Then the samples were incubated in “Click Chemistry Mix” (101 mM Click-it Click Chemistry Buffer, 1.9 mM CuSO4, 1.9 mg/mL ascorbic acid, 20 μM Alexa Fluor™ 488 azide (Invitrogen)) for 30 min at RT. Afterwards, the cells were harvested as before, washed in 150 μL PBS and dissolved in 150 μL fresh PBS. To measure the resulting fluorescence intensity, the cells were analysed by flow cytometry with an excitation wavelength of 488 nm and an emission wavelength of 525 nm. 40,000 events were measured for each sample. For data analysis, the geometric mean used and a blank (cells treated without O-propargyl puromycin addition) was subtracted of each sample.

### c) Effects of PAT1 Deletion in Fed-Batch Cultivations.

To further validate the observations made in the screenings (Example 3), fed-batch cultivations similar to standard production processes for the respective promoter system were done with Δpat1. CBS2612_vHH_Δpat1 #181x and CBS2612_vHH #4 (reference control) were chosen for this purpose to be cultivated with a linear incremental feed profile. The used bioreactor system allows for usage of four parallel bioreactors, so each strain was cultivated in duplicate. In addition to bioprocess parameters and secreted protein titers, also cellular translation activity was measured. The results obtained in this round of fed-batch cultivations can be seen in Table 8. Values for each duplicate reactor are shown side by side.

TABLE 8
Effect of PAT1 deletion on secretion of recombinant proteins and cellular translation activity in
fed-batch cultivations. The column “time” depicts the time passed after initiation of the
linear incremental glucose feed for each sampling point. Measured values for yeast dry mass (YDM)
and vHH titer of the duplicate cultivations are shown side by side. The average fold changes (FC)
of the vHH yield and the relative translation activity between Δpat1 compared to the parent strain are given.

| | | | | | | FC | Relative |
|---|---|---|---|---|---|---|---|
| | Time | YDM | | vHH titer | | vHH | translation |
| Strain | [h] | [g L⁻¹] | | [mg L⁻¹] | | yield | activity |
| CBS2612_vHH#4 | 4.1 | 32.7 ± 0.5 | 32.9 ± 0.3 | 38.0 ± 1.1 | 37.9 ± 0.9 | — | — |
| | 7.3 | 42.1 ± 0.2 | 42.2 ± 0.4 | 83.4 ± 1.8 | 89.2 ± 1.5 | — | — |
| | 22.8 | 84.4 ± 0.7 | 84.1 ± 0.9 | 44.4 ± 2.0 | 94.4 ± 3.4 | — | — |
| | 30.1 | 111.2 ± 0.3 | 112.0 ± 0.8 | 105.3 ± 0.7 | 123.4 ± 5.1 | — | — |
| | 46.6 | 140.3 ± 1.1 | 139.3 ± 0.6 | 329.5 ± 13.5 | 419.3 ± 6.1 | — | — |
| CBS2612_vHH_Δpat1#18 | 4.1 | 33.1 ± 0.4 | 33.2 ± 0.1 | 45.7 ± 1.6 | 47.4 ± 2.1 | 1.2 | 2.5 |
| | 7.3 | 42.1 ± 0.4 | 41.1 ± 0.3 | 95.7 ± 1.3 | 92.3 ± 4.7 | 1.1 | — |
| | 22.8 | 81.9 ± 0.4 | 80.0 ± 0.7 | 459.1 ± 5.1 | 518.4 ± 4.5 | 7.3 | 3.8 |
| | 30.1 | 106.5 ± 1.2 | 104.9 ± 0.4 | 987.8 ± 15.9 | 935.9 ± 58.5 | 8.9 | — |
| | 46.6 | 135.9 ± 1.8 | 130.8 ± 0.7 | 1475.3 ± 15.5 | 1500.2 ± 33.1 | 4.2 | 5.5 |

These results confirmed the beneficial effect of Δpat1 on recombinant protein secretion seen in the previous screening experiments (Example 3). Both, titers and yields, could be increased around 4-fold by the end of the cultivation. Additionally, relative translation activity was shown to be increased in Δpat1 as well, corresponding to the increase in yield. This means, deletion of PAT1 leads to an increase of recombinant protein secretion likely through a decrease of mRNA degradation and therefore an increase of cellular translation activity.

d) Fed-Batch Cultivations with a PAT1 Deletion Strain Producing HAS

To further confirm the results seen for Δpat1 also HSA producing strains were used for a round of fed-batch cultivations. This was done as described in Example 4a except that the equation for the linear incremental feed was: F[mL h⁻¹]=0.02*t+4. This slower feed meant that the batch working volume had to be increased to 0.5 L and bigger reactors (1.8 L benchtop bioreactors (SR15000DLS; Dasgip, Germany)) had to be used. Samples were taken as described in Example 4a for titer and YDM measurement. Additionally, also relative translation activity was measured, as described in Example 4b. The results of the fed-batch cultivations can be found in Table 9. The yields and translation activity of one reference strain, CBS7435_P_{G1-3}_HSA #2 were used to calculate the relative values for all other strains.

TABLE 9
Effect of PAT1 deletion on secretion of HSA and cellular translation activity in fed-batch
cultivations. The column “time” depicts the time passed after initiation of the linear
incremental glucose feed for the specific sampling point. Yeast dry mass (YDM) and vHH
titer were determined at these sampling points. The fold change of the HSA yield and the
relative translation activity for Δpat1 compared to one reference strain (#2) are shown.

| | | | HSA | FC | Relative |
|---|---|---|---|---|---|
| | Time | YDM | titer | vHH | translation |
| Strain | [h] | [g L⁻¹] | [mg L⁻¹] | yield | activity |
| CBS7435_P_{G1-3}_HSA#2 | 3.9 | 28.1 ± 0.9 | 6.8 ± 0.4 | — | — |
| | 9.2 | 38.0 ± 0.6 | 21.5 ± 0.4 | — | — |
| | 32.5 | 71.0 ± 0.3 | 97.1 ± 3.4 | — | — |
| | 48.8 | 90.0 ± 0.4 | 144.3 ± 3.2 | — | — |
| | 70.9 | 103.8 ± 2.7 | 173.2 ± 6.0 | — | — |
| CBS7435_Δpat1#50_ | 3.9 | 30.0 ± 0.3 | 9.5 ± 0.1 | 1.3 | 2.7 |
| P_{G1-3}_HSA#5 | 9.2 | 39.5 ± 0.6 | 27.4 ± 0.5 | 1.2 | — |
| | 32.5 | 72.9 ± 0.2 | 117.7 ± 2.5 | 1.1 | 3.3 |
| | 48.8 | 92.2 ± 0.5 | 181.6 ± 0.1 | 1.2 | 2.6 |
| | 70.9 | 107.7 ± 1.1 | 232.6 ± 4.3 | 1.3 | — |

These bioreactor cultivations showed that Δpat1 strains, also when expressing a different model protein, have a positive impact on productivity, which was increased 1.34-fold at the end of the cultivation. The lower increase could be associated to the fact that the model protein HSA is prone to proteolytic degradation, which partly masks the positive effect of the deletion (Kobayashi et al. J. Biosci. Bioeng. 2000, 89, 55-61). It can be assumed this also happened here. Indeed, Δpat1 strains again show increased translation activity (up to 3-fold), meaning the overall translation activity is more representative in terms cellular protein production.

Overall, deletion of PAT1 could be clearly proven to have a significant positive effect on the production and secretion of different recombinant proteins.

### Example 5: Effect of Decapping Factor Deletion Combined with Translation Factor Overexpression

To test the combinatory effect of these two translation-activity-influencing cell engineering strategies, the translation factor overexpression cassette was transformed into the generated CBS7435_Δpat1lx_vHH #47 and #50 clones as described in Example 1c. In the next step, the generated clones are controlled for improvements of secreted vHH production as described in Example 2. For this, the appropriate comparison strains are cultivated on the same plates as the newly generated ones to ensure comparability.

### Example 6: Construction of PATL1 KO Cell Line

The examples below demonstrate that deletion of the decapping factor PATL1 leads to higher titre of recombinant proteins in a mammalian cell line (CHO).

### a) Generation of Reporter Cell Line

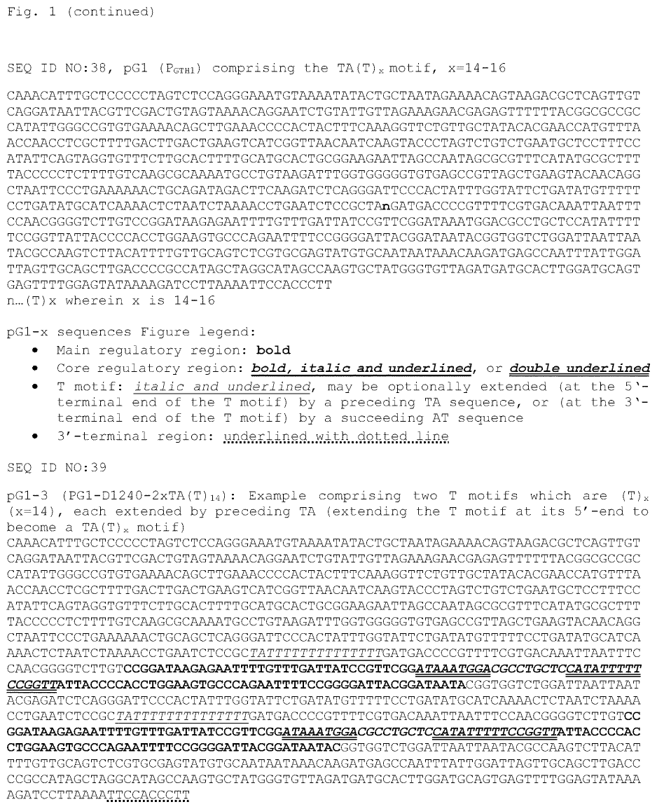

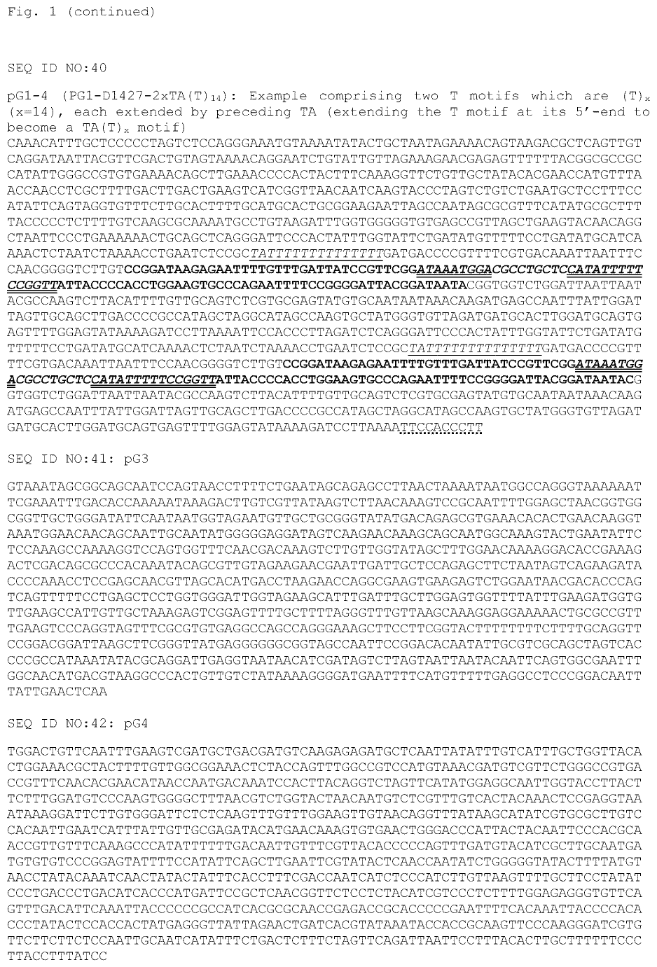

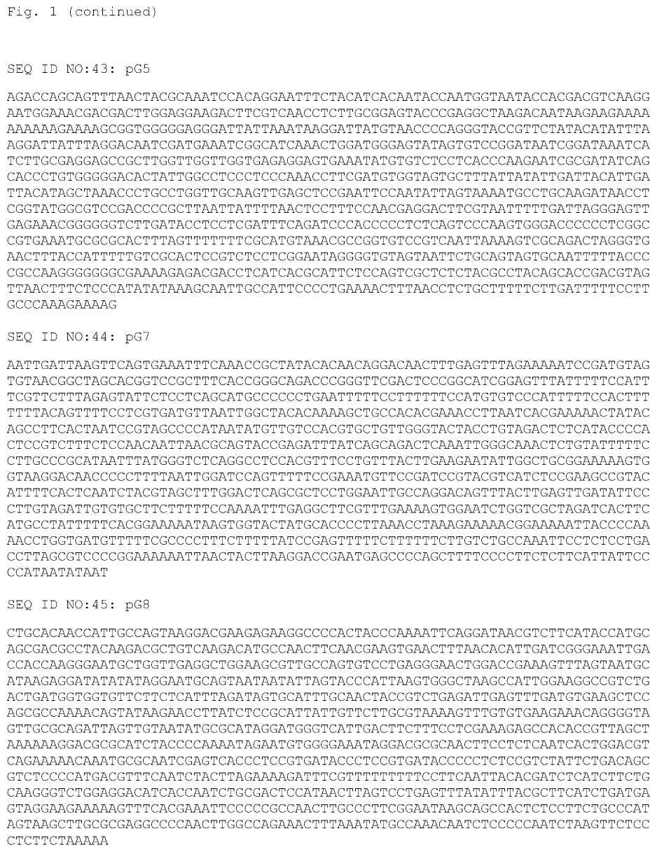

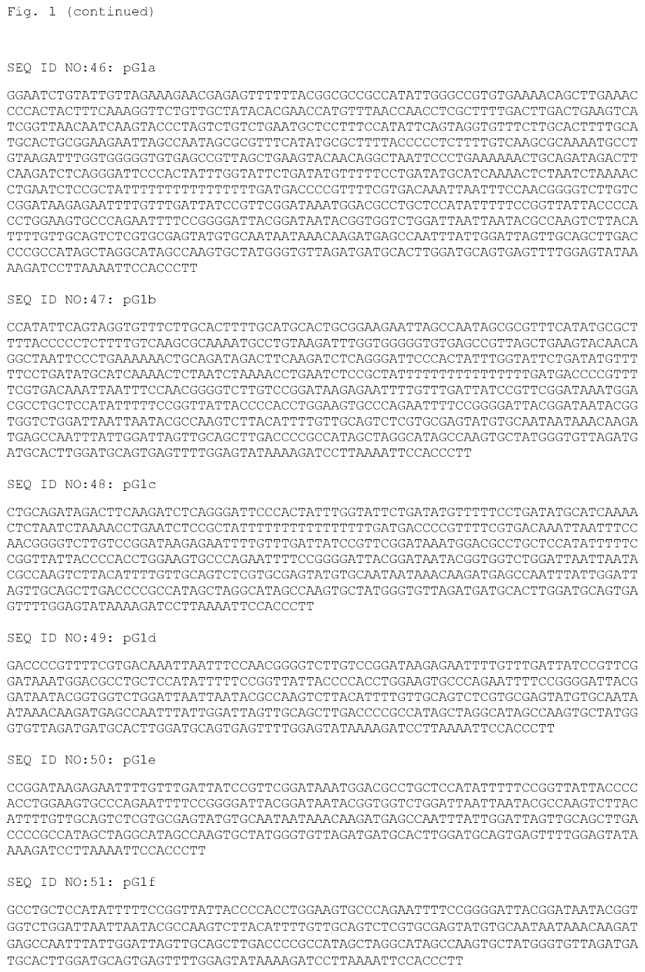

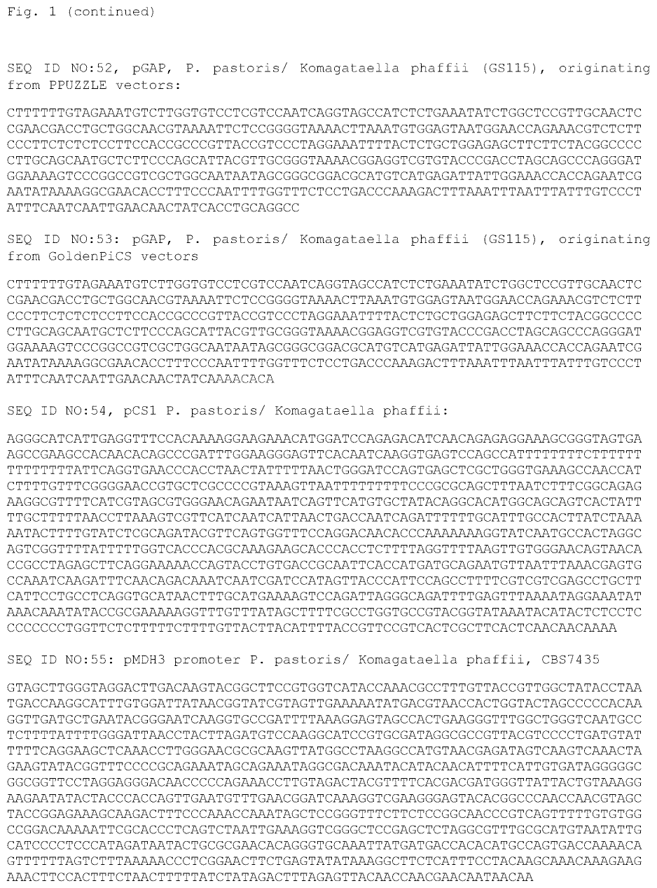

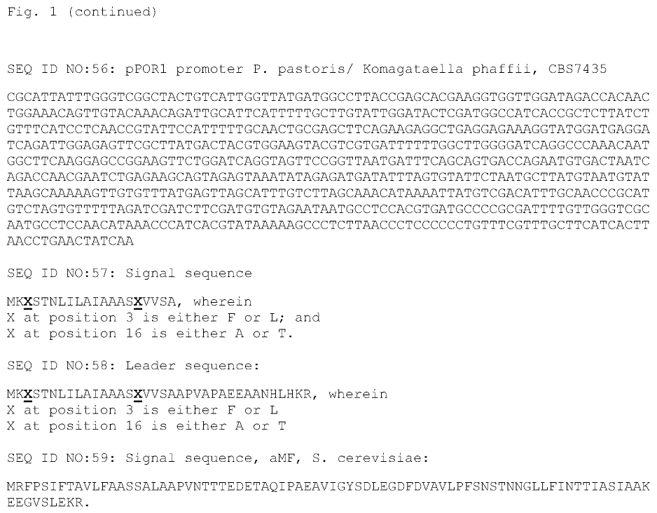

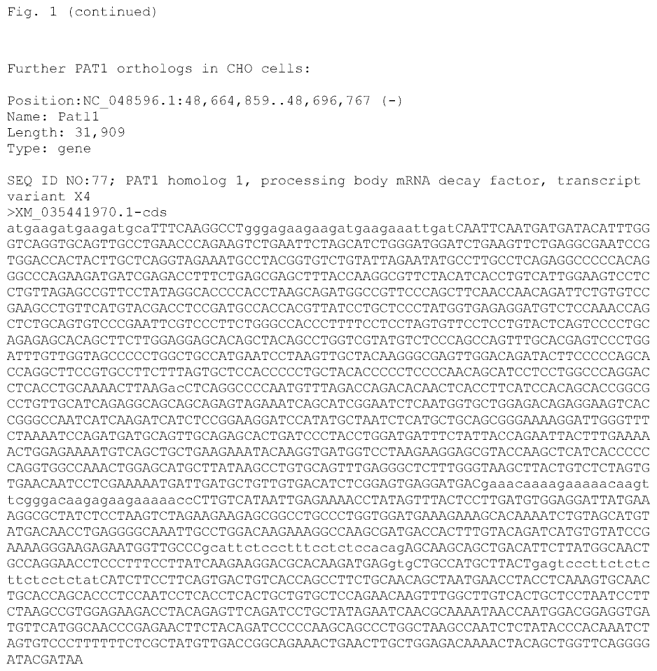

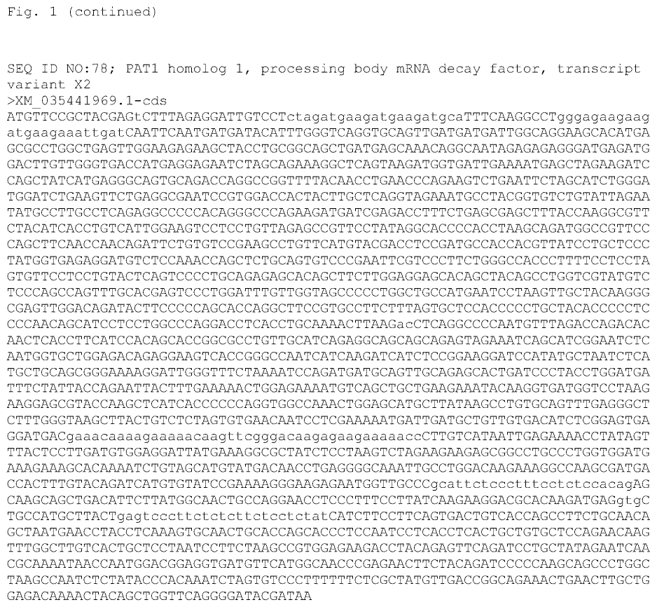

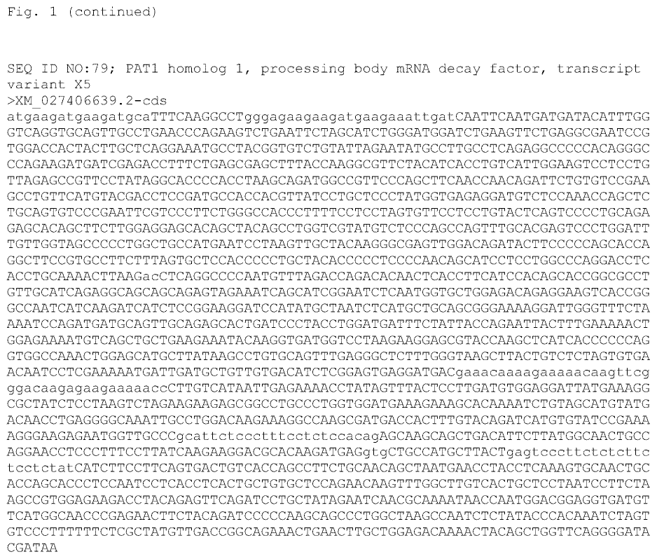

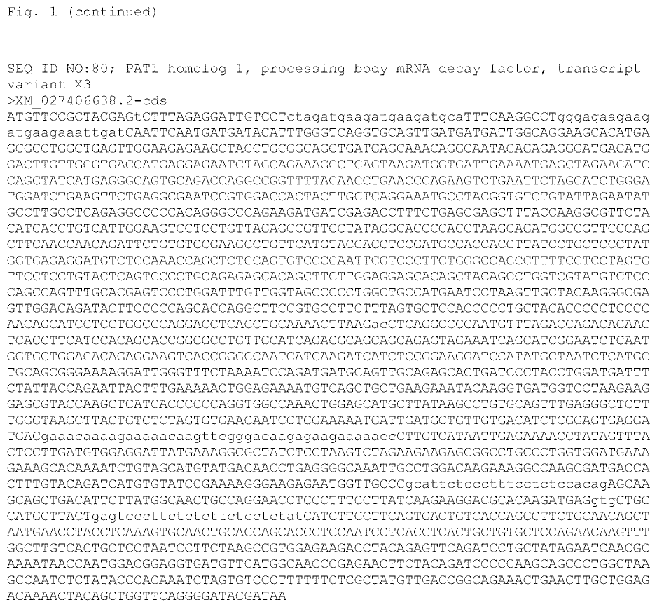

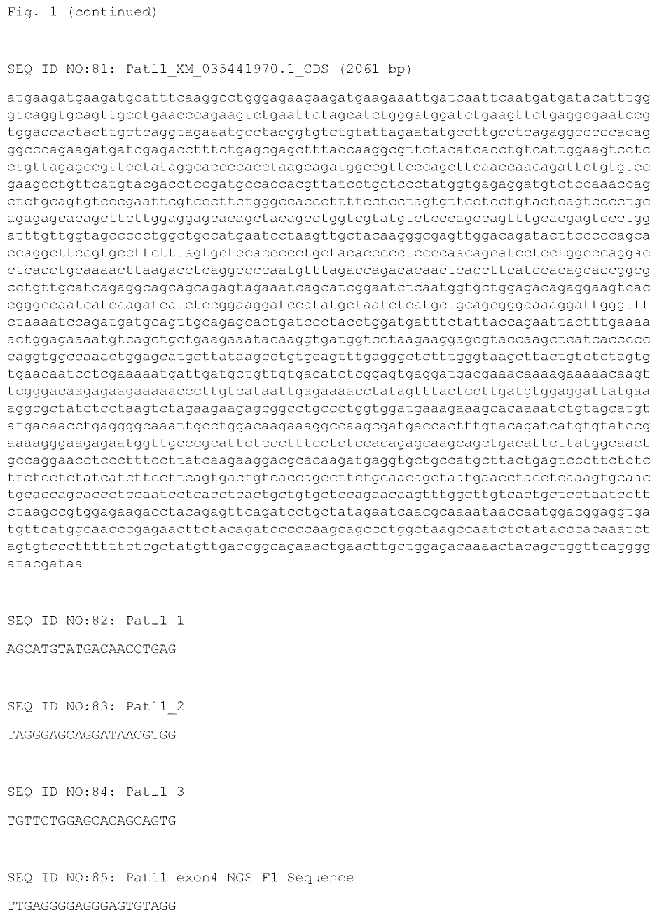

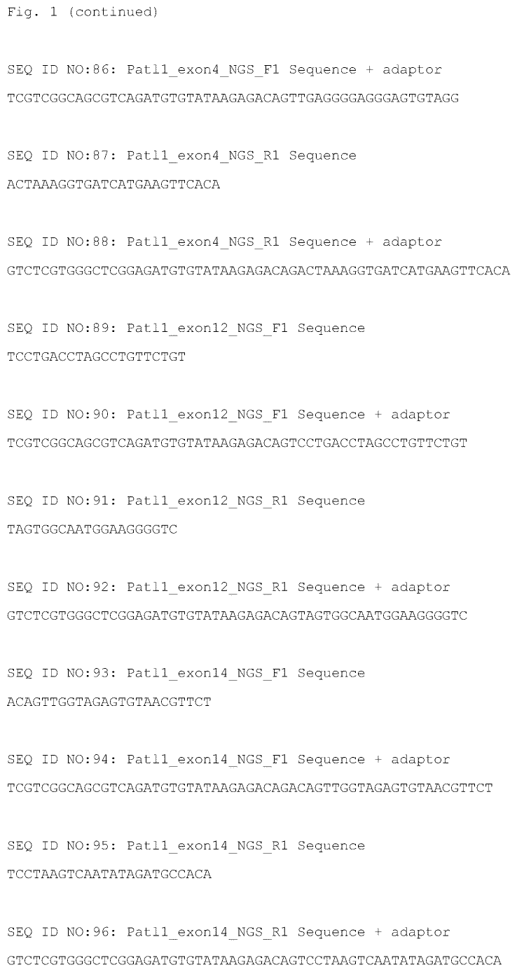

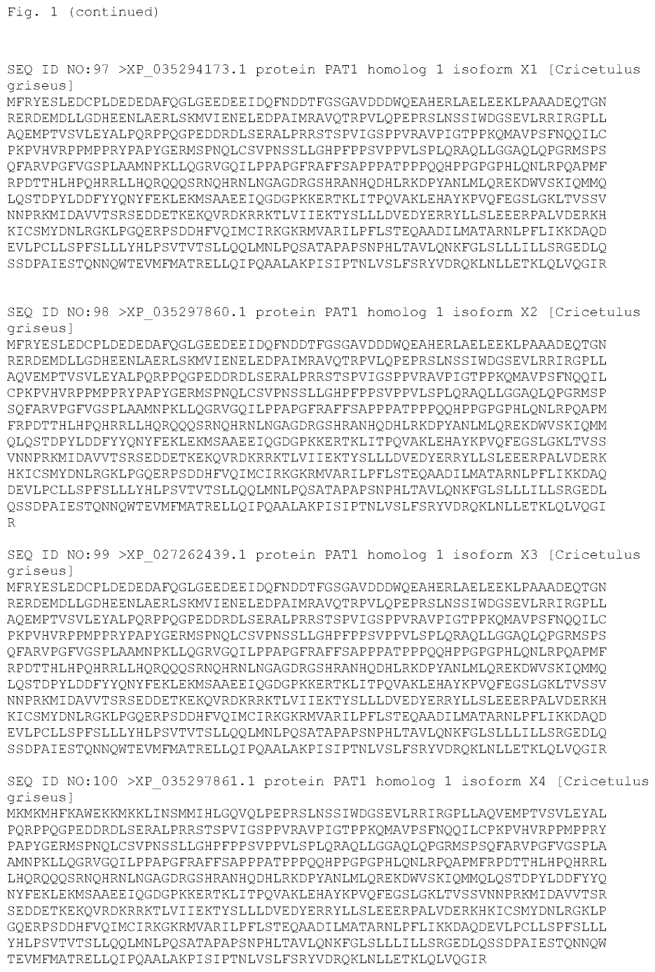

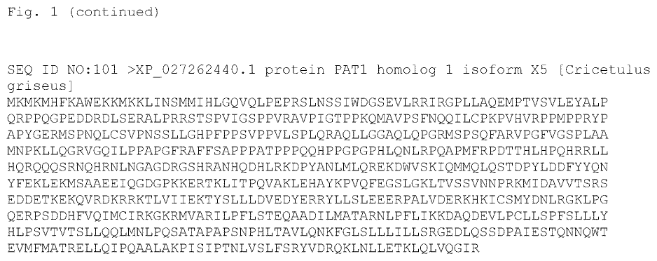

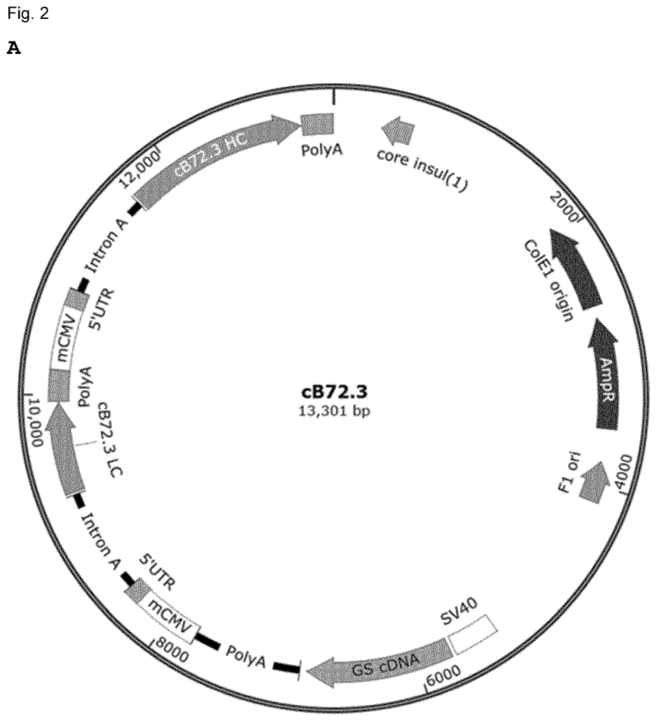

CHOKISV cells (host construction summarized in Fan et al. (Journal of Biotechnology 2013; 168 (4):652-658), were used as parental cell line. Knockout of the PATL1 gene was performed using CRISPR/Cas system. Antibody (cB72.3, a Chimeric mouse/human antibody B72.3 (Colcher et al., 1989, Cancer Res (1989) 49 (7): 1738-1745), an antibody (cB72.3 mAb) that recognises tumour associated glycoprotein TAG-72)) and Cas9 expressing reporter cell lines has been constructed and was used to test the impact of the PATL1 KO. Cas9 plasmid was under Blasticidin selection and antibody construct was under GS selection (vector maps in FIGS. 2A and B). Cells were first transfected with antibody expressing vectors by electroporation) and were subjected to MSX selection. An antibody expressing cell line pool that was selected for stable expression was further transduced with lentivirus vectors containing Cas9 expression cassette and cells were selected in Blasticidin. Cells with dual selection were taken forward for host cell construction.

b) Generation of PATL1 KO cell line.

To generate the PATL1 KO strain, Cas9 guides were designed for PATL1 gene (SEQ ID NO:81) using CRISPRseek algorithm (Bioconductor version 3.13, Zhu et al., 2014,). Top 3 guides with high on target activity and low off target activity was chosen. Guides were targeting exon 4, 12 and 14 of the gene (illustrated in FIG. 3 and guide sequence in Table 10). Reporter cell line was transfected with all 3 guides in a multiplexed fashion. Reporter cell line treated with TE was used as no guide control. Combination of 3 guides (that has no targets in the CHO genome) was used as non-targeting control. All transfected cells were further expanded and characterised genotypically and phenotypically
1) Genotyping to assess the KO efficiency
2) Secretory readout by Octet® (Sartorius) using protein A biosensors.

TABLE 10
Guide sequences used to generate deletion of
gene PATL1 (SEQ ID NO: 82-84)

| Name | Sequence |
|---|---|
| Patl1_1 | AGCATGTATGACAACCTGAG (SEQ ID NO: 82) |
| Patl1_2 | TAGGGAGCAGGATAACGTGG (SEQ ID NO: 83) |
| Patl1_3 | TGTTCTGGAGCACAGCAGTG (SEQ ID NO: 84) |

### c) Genotyping to Assess PATL1 KO Pools

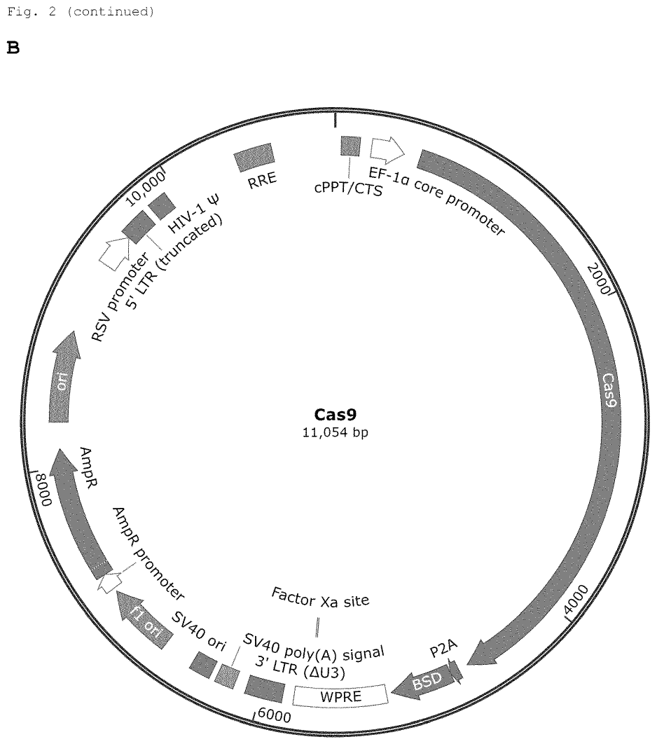

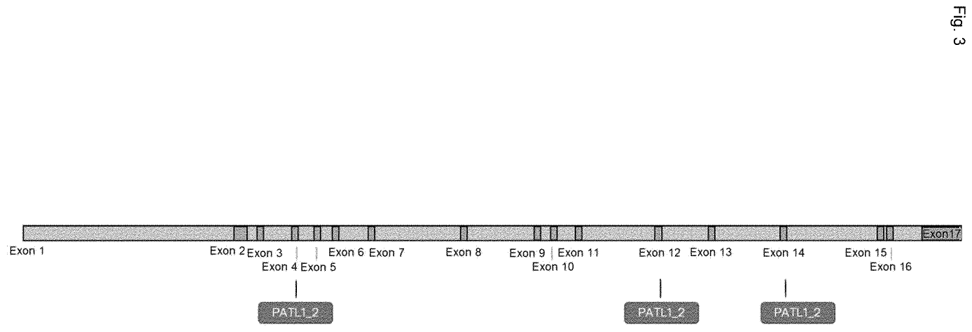

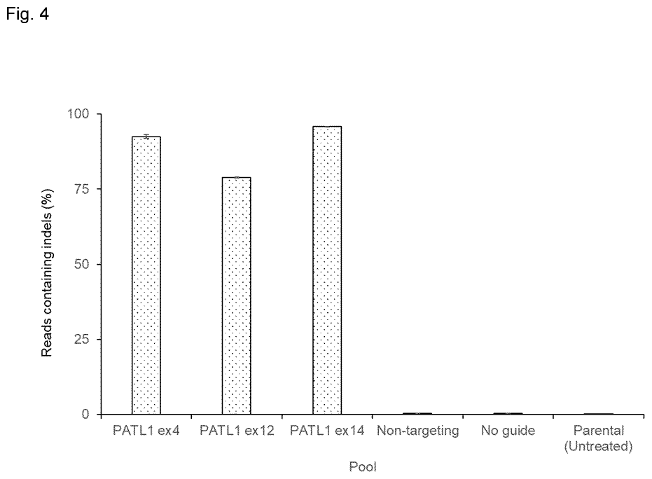

Transfected cells were collected 3 days post transfection to extract genomic DNA. Edited region of PATL1 was amplified using specific primers (sequence details in Table 11). PCR products were further subjected to NGS library prep for amplicon sequencing using standard illumina protocol. Primers used for library amplification is included in Table 11. Libraries were sequenced in Miseq platform (paired end 250 bp). Analysis of the NGS of amplicons was carried out using CRISPResso version 1 software (GitHub, Inc.; Pinello et al. (Nat Biotechnol 2016; 34(7):695-7; https://github.com/lucapinello/CRISPResso) showing >80% (indels) editing on all three edited region of the gene compared to low levels in untreated and non-targeting controls (FIG. 4).

TABLE 11
Primer sequences used for PCR amplification and NGS sequencing
(SEQ ID NO: 85-96)

| | | | | Amplicon |
|---|---|---|---|---|
| | | | Amplicon | size + |
| | | | size | adaptor |
| Primer ID | Sequence (5′-3′) | Sequence + adaptor (5′-3′) | (bp) | (bp) |
| Patl1_exon | TTGAGGGGAGG | TCGTCGGCAGCGTCAGATGTGTATAAG | 223 | 290 |
| 4_NGS_F1 | GAGTGTAGG | AGACAGTTGAGGGGAGGGAGTGTAGG | | |
| | (SEQ ID NO: 85) | (SEQ ID NO: 86) | | |
| Patl1_exon | ACTAAAGGTGA | GTCTCGTGGGCTCGGAGATGTGTATAA | 223 | 290 |
| 4_NGS_R1 | TCATGAAGTTCA | GAGACAGACTAAAGGTGATCATGAAGTT | | |
| | CA | CACA (SEQ ID NO: 88) | | |
| | (SEQ ID NO: 87) | | | |
| Patl1_exon | TCCTGACCTAG | TCGTCGGCAGCGTCAGATGTGTATAAG | 237 | 304 |
| 12_NGS_F1 | CCTGTTCTGT | AGACAGTCCTGACCTAGCCTGTTCTGT | | |
| | (SEQ ID NO: 89) | (SEQ ID NO: 90) | | |
| Patl1_exon | TAGTGGCAATG | GTCTCGTGGGCTCGGAGATGTGTATAA | 237 | 304 |
| 12_NGS_ | GAAGGGGTC | GAGACAGTAGTGGCAATGGAAGGGGTC | | |
| R1 | (SEQ ID NO: 91) | (SEQ ID NO: 92) | | |
| Patl1_exon | ACAGTTGGTAG | TCGTCGGCAGCGTCAGATGTGTATAAG | 248 | 315 |
| 14_NGS_F1 | AGTGTAACGTT | AGACAGACAGTTGGTAGAGTGTAACGTT | | |
| | CT | CT (SEQ ID NO: 94) | | |
| | (SEQ ID NO: 93) | | | |
| Patl1_exon | TCCTAAGTCAAT | GTCTCGTGGGCTCGGAGATGTGTATAA | 248 | 315 |
| 14_NGS_R1 | ATAGATGCCAC | GAGACAGTCCTAAGTCAATATAGATGCC | | |
| | A | ACA (SEQ ID NO: 96) | | |
| | (SEQ ID NO: 95) | | | |

### d) Phenotyping to Assess Effects of PATL1 Deletion on Recombinant Protein Production at Pool Level

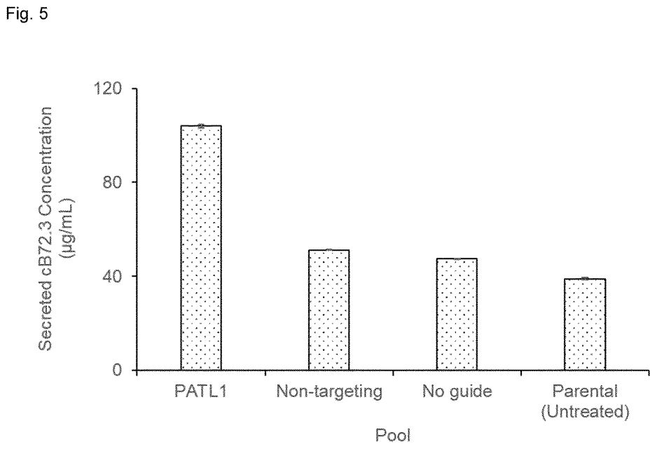

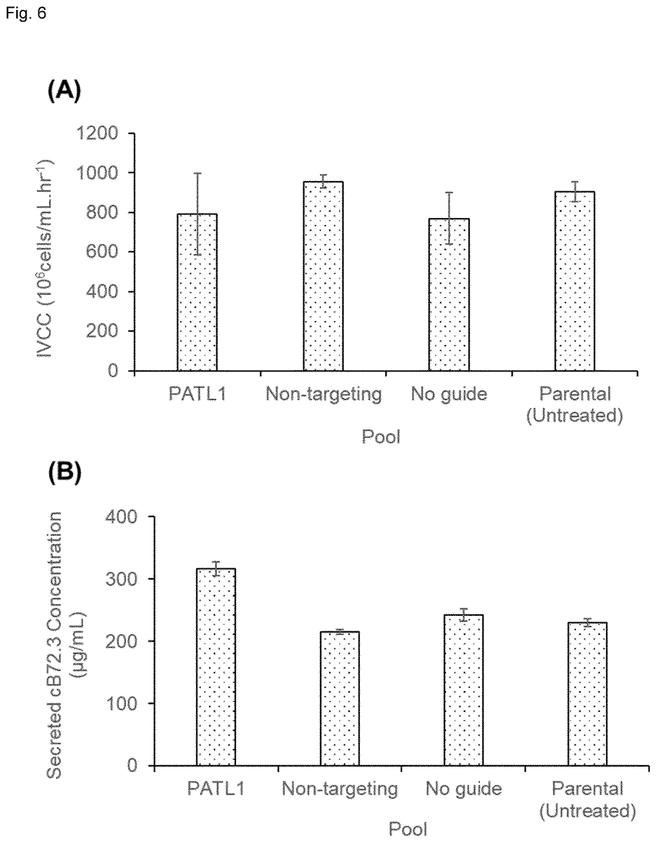

To determine the effect of PATL1 deletion on recombinant protein secretion, secretory expression was analysed in KO cell lines post transfection. Supernatant was collected from KO pools and the recombinant protein secreted into the supernatant was quantified using Octet® (Sartorius) using protein A biosensors. PATL1 KO pools showed an increase of >50% titre compared to untreated and non-targeting controls (FIG. 5). We further extended the pool characterisation to a 10-day batch culture. Batch was performed on cells seeded at same density in CD CHO medium (Gibco). Cell growth was monitored on days 4, 7 and 10. Secreted product accumulated over the time was measured by Octet® (Sartorius) using protein A biosensors on Day 10 (FIG. 6). PATL1 KO pools consistently showed enhanced recombinant protein production by improving the titre to >30% (FIG. 6).

### e) Functional Characterisation of PATL1 KO Pools

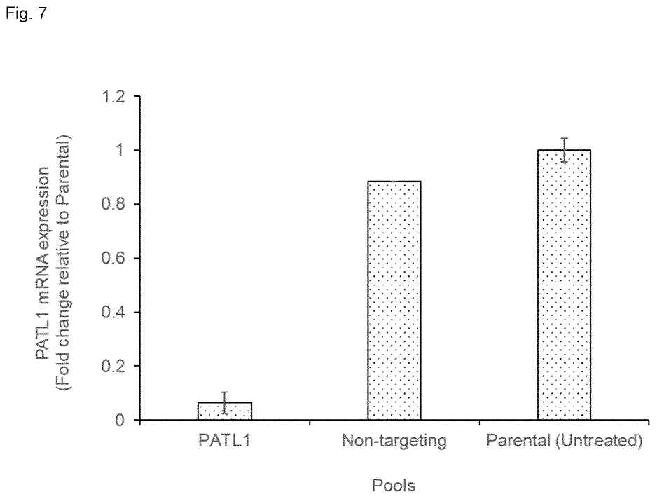

In order to investigate that the titre increase is an effect from PA TL1 deletion itself, transcript level of the PATL1 gene was assessed by qPCR in controls and PATL1 KO pools. The pools were pelleted and RNA was isolated using (Single shot SyBR green one step kit from Biorad). qPCR was performed on controls and PATL1 KO pools using specific primers to detect PATL1 transcript. GAPDH was used as housekeeping gene control. Ct values obtained for PATL1 were normalised with GAPDH and fold change relative to parental are presented on plots (FIG. 7). PATL1 transcript level was significantly reduced in the KO pools compared to untreated and non-targeting controls.

### Example 7: Characterisation at Clone Level

The investigation was further extended from pool level to clone level. PATL1 KO pools were sorted into single cells by Beacon® Optofluidic System (Bruker). The selected and exported clones were expanded for functional characterisation and phenotypic assessment. Cells were maintained in a 96 well format and confluency was assessed by Celigo Image Cytometer (Nexcelom).

### a) Phenotypic Assessment of PATL1 Deletion on Recombinant Protein Production at Clone Level

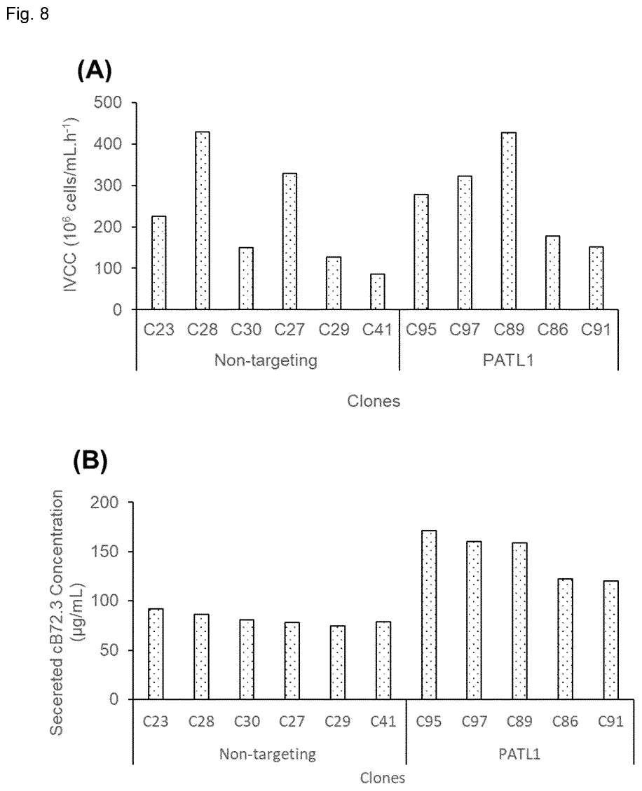

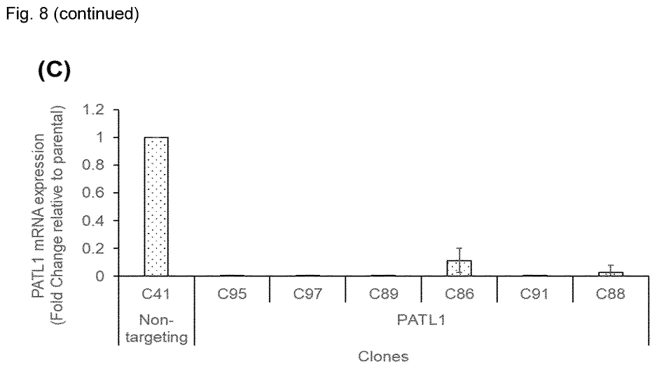

To determine the effect of PATL1 deletion on recombinant protein secretion at clone level, secretory expression was analysed in isolated KO clones. For this a 10-day batch culture was performed in a DWP format on cells seeded at same density in CD CHO medium (Gibco). Cell growth was monitored on days 4, 7 and 10. Secreted product accumulated over the time was measured by Octet® (Sartorius) using protein A biosensors on Day 10 and IVCC are shown (FIGS. 8 A and B, respectively). The majority of the PATL1 KO clones exhibited enhanced recombinant protein production by improving the titre between >30% to >60% (FIG. 8 B).

### b) Functional Characterisation of PATL1 KO Clones

Functional characterisation of transcript level KO was also extended to clones similar to that was performed in pools. In order to investigate that the titre increase is an effect from PATL1 deletion itself, transcript level of the PATL1 gene was assessed by qPCR in controls and all the KO clones. Cells were pelleted and cell lysate was prepared (Single shot SyBR green one step kit from Biorad). qPCR was performed on controls and PATL1 KO pools using specific primers and lysate as input to detect PATL1 transcript. GAPDH was used as housekeeping gene control. Ct values obtained for PATL1 were normalised with GAPDH and fold-change relative to non-targeting were represented as plots (FIG. 8 C). PATL1 transcript level was significantly reduced in the KO clones compared to non-targeting control.

1. A eukaryotic host cell which is engineered to introduce a recombinant expression cassette expressing a gene of interest (GOI) to produce a protein of interest (POI), and by a genetic modification to control expression of a Pat1 protein.

2. The host cell of claim 1, wherein the level of the Pat1 protein is controlled by said genetic modification, and wherein controlling the level of the Pat1 protein increases the POI yield and/or titer.

3-6. (canceled)

7. The host cell of claim 1, wherein the expression of the Pat1 protein is reduced compared to an endogenous expression thereof.

8. The host cell of claim 7, wherein said genetic modification comprises a disruption, substitution, deletion or knockout of (i) an endogenous polynucleotide encoding the Pat1 protein, or a part thereof; or (ii) an expression control sequence that controls expression of a polynucleotide encoding the Pat1 protein, wherein the polynucleotide encoding the Pat1 protein is endogenous to the host cell.

9-11. (canceled)

12. The host cell of claim 1, wherein the expression cassette comprises one or more expression control sequences operably linked to said GOI.

13. The host cell of claim 12, wherein said one or more expression control sequences comprise a promoter which is an inducible, de-repressible or otherwise regulatable promoter, or a constitutive promoter.

14. (canceled)

15. (canceled)

16. The host cell of claim 1, wherein the POI is a therapeutic or diagnostic product.

17. The host cell of claim 16, wherein the POI is a therapeutic product selected from the group consisting of an antigen-binding protein, an enzyme, a peptide, a protein antibiotic, a toxin fusion protein, a structural protein, a regulatory protein, a protein vaccine antigen, a hormone, a growth factor, a cytokine, and a blood clotting or coagulation factor.

18. The host cell of claim 17, wherein the antigen-binding protein is an antibody molecule, wherein the antibody molecule is a monoclonal antibody, a full-length antibody, an antibody comprising one or more epitope binding fragments of a full-length antibody, or a bispecific or multi-specific antibody comprising one or more of said fragments, wherein said one or more epitope binding fragments of a full-length antibody are Fab, Fab′, F(ab′)₂, Fv, or scFv fragments, or single domain antibodies.

19-21. (canceled)

22. The host cell of claim 1, wherein the POI is a process enzyme or metabolic enzyme.

23. The host cell of claim 1, wherein the Pat1 protein is Pat1 comprising SEQ ID NO:1, SEQ ID NO:3, any one of SEQ ID NOs:13 to 17, or any one of SEQ ID NOs:97to 101, or a Pat1 ortholog to any one of the foregoing, which is encoded by a host cell endogenous polynucleotide without said genetic modification to control expression of said Pat1 protein, wherein the host cell endogenous polynucleotide is endogenous to the wild-type host cell.

24. (canceled)

25. (canceled)

26. The host cell of claim 1, wherein the host cell is any one of Pichia pastoris, Saccharomyces cerevisiae, Yarrowia lipolytica, Ogataea polymorpha, Schizosaccharomyces pombe, Trichoderma reesei, Kluyveromyces lactis, Aspergillus niger, Homo sapiens, or Cricetulu griseus host cells, wherein the Pichia pastoris is selected from Komagataella phaffii, Komagataella pastoris, and Komagataella pseudopastoris.

27. The host cell of claim 1, wherein:
a) the host cell is a Komagataella phaffii host cell, and the Pat1 protein comprises at least 90% sequence identity to SEQ ID NO:1;
b) the host cell is a Komagataella pastoris host cell, and the Pat1 protein comprises at least 90% sequence identity to SEQ ID NO:3;
c) the host cell is a Saccharomyces cerevisiae host cell, and the Pat1 protein comprises at least 90% sequence identity to SEQ ID NO:4;
d) the host cell is a Yarrowia lipolytica host cell, and the Pat1 protein comprises at least 90% sequence identity to SEQ ID NO:5;
e) the host cell is an Ogataea polymorpha host cell, and the Pat1 protein comprises at least 90% sequence identity to SEQ ID NO:6;
f) the host cell is a Schizosaccharomyces pombe host cell, and the Pat1 protein comprises at least 90% sequence identity to SEQ ID NO:7;
g) the host cell is a Trichoderma reesei host cell, and the Pat1 protein comprises at least 90% sequence identity to SEQ ID NO:8 h) the host cell is a Kluyveromyces lactis host cell, and the Pat1 protein comprises at least 90% sequence identity to SEQ ID NO:9;
i) the host cell is an Aspergillus niger host cell, and the Pat1 protein comprises at least 90% sequence identity to SEQ ID NO:10,
j) the host cell is a Homo sapiens host cell, and the Pat1 protein comprises at least 90% sequence identity to SEQ ID NO:11 or 12; or
k) the host cell is a Cricetulu griseus host cell, and the Pat1 protein comprises at least 90% sequence identity to any one of SEQ ID NOs: 13 to 17, or any one of SEQ ID NOs:97 to 101.

28. (canceled)

29. The host cell of claim 1, wherein the host cell is a P. pastoris or CHO cell.

30. A method for producing a host cell of claim 1, comprising genetic engineering of a host cell to (i) introduce said recombinant expression cassette expressing said GOI, and to (ii) control expression of a Pat1 protein.

31-35. (canceled)

36. The method of claim 30, wherein the genetic engineering comprises knocking out at least one or all copies of a gene encoding the Pat1 protein from the genome of the cell or a regulatory nucleic acid sequence operably linked to the gene encoding the Pat1 protein, by a split-marker cassette method, or by a method using a CRISPR-Cas9 protein in combination with an RNA specific for the gene encoding the Pat1 protein or specific to the regulatory nucleic acid sequence.

37. A method of increasing the yield of producing a heterologous protein of interest (POI) in a cell culture of a host cell under carbon source limiting conditions, by engineering the host cell to control expression of a Pat1 protein in the host cell.

38. The method of claim 37, wherein the carbon-source limiting conditions are characterized by the presence of a growth-limiting amount of a carbon source in the host cell culture, which is up to 1 g/L.

39. The method of claim 37, wherein the yield is at least 1.2-fold increased compared to the host cell without said genetic modification to control expression of the Pat1 protein.

40-41. (canceled)

42. The method of claim 37, wherein the expression of the Pat1 protein is reduced compared to an endogenous expression thereof.

43. The method of claim 42, wherein the engineering comprises knocking out at least one or all copies of a gene encoding the Pat1 protein from the genome of the cell or a regulatory nucleic acid sequence operably linked to the gene encoding the Pat1 protein, by a split-marker cassette method, or by a method using a CRISPR-Cas9 protein in combination with an RNA specific for the gene encoding the Pat1 protein or specific to the regulatory nucleic acid sequence.

44. The method of claim 37, wherein the host cell is a P. pastoris host cell, wherein the expression of the Pat1 protein is reduced by inactivating expression of the Pat1 protein in the host cell.

45. (canceled)

46. A method for producing a protein of interest (POI) in a host cell culture, by culturing the host cell of claim 1 under conditions to produce said POI.

47. The method of claim 46, wherein the host cell is cultured in a host cell culture under carbon-source limiting conditions to produce said POI.

48. The method of claim 46, wherein the carbon-source limiting conditions are characterized by the presence of a growth-limiting amount of a carbon source in the host cell culture, which is up to 1 g/L.

49. (canceled)

50. (canceled)

51. A pat1 knockout eukaryotic host cell producing a protein of interest (POI) at an at least 1.2-fold increased yield and/or titer and/or specific productivity (μg/g cell dry mass (CDM)) per hour and/or volumetric productivity (μg/L per hour) compared to the host cell without said pat1 knockout, wherein:
a) the host cell is a Komagataella phaffli host cell, and the Pat1 protein comprises at least 90% sequence identity to SEQ ID NO:1:
b) the host cell is a Komaataella pastoris host cell, and the Pat1 protein comprises at least 90% sequence identity to SEQ ID NO:3:
c) the host cell is a Saccharomyces cerevisiae host cell, and the Pat1 protein comprises at least 90% sequence identity to SEQ ID NO:4;
d) the host cell is a Yarrowia lipolytica host cell, and the Pat1 protein comprises at least 90% sequence identity to SEQ ID NO:5;
e) the host cell is an Ogataea polymorpha host cell, and the Pat1 protein comprises at least 90% sequence identity to SEQ ID NO:6:
f) the host cell is a Schizosaccharomyces pombe host cell, and the Pat1 protein comprises at least 90% sequence identity to SEQ ID NO:7;
g) the host cell is a Trichoderma reesei host cell, and the Pat1 protein comprises at least 90% sequence identity to SEQ ID NO:8;
h) the host cell is a Kluyveromyces lactis host cell, and the Pat1 protein comprises at least 90% sequence identity to SEQ ID NO:9:
i) the host cell is an Aspergillus niger host cell, and the Pat1 protein comprises at least 90% sequence identity to SEQ ID NO:10;
j) the host cell is a Homo sapiens host cell, and the Pat1 protein comprises at least 90% sequence identity to SEQ ID NO:11 or 12: or
k) the host cell is a Cricetulu griseus host cell, and the Pat1 protein comprises at least 90% sequence identity to any one of SEQ ID NOs:13 to 17, or any one of SEQ ID NOs:97 to 101.

52-61. (canceled)

62. The pat1 knockout eukaryotic host cell of claim 51, wherein the POI is a therapeutic or diagnostic product.

63. The pat1 knockout eukaryotic host cell of claim 62, wherein the POI is a therapeutic product selected from the group consisting of an antigen-binding protein, an enzyme, a peptide, a protein antibiotic, a toxin fusion protein, a structural protein, a regulatory protein, a protein vaccine antigen, a hormone, a growth factor, a cytokine, and a blood clotting or coagulation factor.

64. The pat1 knockout eukaryotic host cell of claim 63, wherein the antigen-binding protein is an antibody molecule, wherein the antibody molecule is a monoclonal antibody, a full-length antibody, an antibody comprising one or more epitope binding fragments of a full-length antibody, or a bispecific or multi-specific antibody comprising one or more of said fragments, wherein said one or more epitope binding fragments of a full-length antibody are Fab, Fab′, F(ab′)₂, Fv, or scFv fragments, or single domain antibodies.

65-67. (canceled)

68. The pat1 knockout eukaryotic host cell of claim 51, wherein the POI is a process enzyme or metabolic enzyme.

69. The host cell of claim 13, wherein the promoter is any one of SEQ ID NO:38-51, or any one of a CMV, SV40 or PGK promoter.
